SEQUENCE LISTING (58 sequences)

SEQ ID NO: 1 - DNA, 381 bp, Artificial Sequence
  FHF11 VH (wt-nt)
caggtacaactgcagcagtcaggtccaggactggtgaagccctcgcagaccctctcagtcacctgtggcatctccggggacagtgtctctagtcacagtgctgcttggaactggatcaggcagtccccatcgagaggccttgagtggctgggaaggacatattacaggtccaagtggtataatgattatgcagtctctgtgaaaagtcgaataaccatcaatccagacacatccaagaaccagttctccctacagttgatctctgtgactcccgaggacacggctgtctattactgtgcaagagtgggtgctatgacttttggacttcttacagggggtatggacgtctggggccaagggaccacggtcaccgtctcctca

SEQ ID NO: 2 - protein, 127 aa, Artificial Sequence
  FHF11 VH (aa)
QVQLQQSGPGLVKPSQTLSVTCGISGDSVSSHSAAWNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 3 - protein, 10 aa, Artificial Sequence
  FHF11 CDR-H1 (aa)
GDSVSSHSAA

SEQ ID NO: 4 - protein, 9 aa, Artificial Sequence
  FHF11 CDR-H2 (aa)
TYYRSKWYN

SEQ ID NO: 5 - protein, 17 aa, Artificial Sequence
  FHF11 CDR-H3 (aa)
ARVGAMTFGLLTGGMDV

SEQ ID NO: 6 - DNA, 381 bp, Artificial Sequence
  FHF11 VH (co-nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 7 - DNA, 328 bp, Artificial Sequence
  FHF11 Vk (wt-nt)
gaaattgtgttgacgcagtctccaggcacccagtctttgtctccaggggaaagagccaccctctcctgcagggccagtcagagtctgagccgcagctacttagcctggtaccagcagagacctggcaagcctcccaggctcctcatctatggtgcatccagcagggccactggcatcccagacaggttcagtggcagtgggtctgggacagacttcagtctcaccatcagcagtctggagcctgaagattctgcaatgtatttctgtcagtactatggtgattcacctctattcagtttcggcccagggaccaaagtggatatcaaac

SEQ ID NO: 8 - protein, 109 aa, Artificial Sequence
  FHF11 Vk (aa)
EIVLTQSPGTQSLSPGERATLSCRASQSLSRSYLAWYQQRPGKPPRLLIYGASSRATGIPDRFSGSGSGTDFSLTISSLEPEDSAMYFCQYYGDSPLFSFGPGTKVDIK

SEQ ID NO: 9 - protein, 7 aa, Artificial Sequence
  FHF11 CDR-L1 (aa)
QSLSRSY

SEQ ID NO: 10 - protein, 3 aa, Artificial Sequence
  FHF11 CDR-L2 (aa)
GAS

SEQ ID NO: 11 - protein, 10 aa, Artificial Sequence
  FHF11 CDR-L3 (aa)
QYYGDSPLFS

SEQ ID NO: 12 - DNA, 327 bp, Artificial Sequence
  FHF11 Vk (co-nt)
gagatcgtgctgacccagtctcctggcacacagagcctgtctccaggagagagggccaccctgtcctgcagggcttcccagagcctgtctaggtcctacctggcctggtatcagcagagaccaggcaagccacctaggctgctgatctacggagcttccagcagggctacaggcatccctgacagattcagcggctctggctccggcaccgatttttccctgacaatctcttccctggagccagaggactccgccatgtatttctgtcagtactatggcgatagcccactgttctcttttggccccggcaccaaggtggacatcaag

SEQ ID NO: 13 - DNA, 381 bp, Artificial Sequence
  FHF12 VH (wt-nt)
caggtacaactgcagcagtcaggtccaggactggtgaagccctcgcagaccctctcagtcacctgtgccatctccggggacagtgtctctagtcacagtgctgcttggaactggatcaggcagtccccatcgagaggccttgagtggctgggaaggacatattacaggtccaagtggtataatgattatgcagtctctgtgaaaagtcgaataaccatcaacccagacacatccaagaaccagttctccctacagctggtctctgtgactcccgaggacacggctgtctattactgtgcaagagtgggtgctgcgacttttggaattcttacagggggtatggacgtctggggccaagggaccacggtcaccgtctcctca

SEQ ID NO: 14 - protein, 127 aa, Artificial Sequence
  FHF12 VH (aa)
QVQLQQSGPGLVKPSQTLSVTCAISGDSVSSHSAAWNWIRQSPSRGLEWLGRTYYRSKWYNDYAVSVKSRITINPDTSKNQFSLQLVSVTPEDTAVYYCARVGAATFGILTGGMDVWGQGTTVTVSS

SEQ ID NO: 15 - protein, 10 aa, Artificial Sequence
  FHF12 CDR-H1 (aa)
GDSVSSHSAA

SEQ ID NO: 16 - protein, 9 aa, Artificial Sequence
  FHF12 CDR-H2 (aa)
TYYRSKWYN

SEQ ID NO: 17 - protein, 17 aa, Artificial Sequence
  FHF12 CDR-H3 (aa)
ARVGAATFGILTGGMDV

SEQ ID NO: 18 - DNA, 381 bp, Artificial Sequence
  FHF12 VH (co-nt)
caggtgcagctgcagcagtctggaccaggactggtgaagcctagccagaccctgtctgtgacatgcgctatctccggcgacagcgtgtccagccactccgccgcttggaactggatcagacagagcccatctaggggactggagtggctgggaaggacctactatcggagcaagtggtacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 19 - DNA, 328 bp, Artificial Sequence
  FHF12 Vk (wt-nt)
gaaattgtgttgacgcagtctccaggcacccagtctttgtctccaggggatagagccaccctctcctgcagggccagtcagagtctgagcagaagctacttagcctggtaccagcagagacctggcaagcctcccaggctcctcatctatggtgcatccagcagggccactggcatcccagacaggttcagtggcagtgggtctgggacagacttcagtctcaccatcagcagtctggagcctgaagattctgctatgtatttctgtcagtactatggtgattcacctctattcagtttcggccctgggaccaaagtggatatcaaac

SEQ ID NO: 20 - protein, 109 aa, Artificial Sequence
  FHF12 Vk (aa)
EIVLTQSPGTQSLSPGDRATLSCRASQSLSRSYLAWYQQRPGKPPRLLIYGASSRATGIPDRFSGSGSGTDFSLTISSLEPEDSAMYFCQYYGDSPLFSFGPGTKVDIK

SEQ ID NO: 21 - protein, 7 aa, Artificial Sequence
  FHF12 CDR-L1 (aa)
QSLSRSY

SEQ ID NO: 22 - protein, 3 aa, Artificial Sequence
  FHF12 CDR-L2 (aa)
GAS

SEQ ID NO: 23 - protein, 10 aa, Artificial Sequence
  FHF12 CDR-L3 (aa)
QYYGDSPLFS

SEQ ID NO: 24 - DNA, 327 bp, Artificial Sequence
  FHF12 Vk (co-nt)
gagatcgtgctgacccagtctcctggcacacagagcctgtctccaggcgacagggccaccctgtcctgcagggcttcccagagcctgtctaggtcctacctggcctggtatcagcagagaccaggcaagccacctaggctgctgatctacggagcttccagcagggctacaggcatccctgacagattcagcggctctggctccggcaccgatttttccctgacaatctcttccctggagccagaggactccgccatgtatttctgtcagtactatggcgatagcccactgttctcttttggccccggcaccaaggtggatatcaag

SEQ ID NO: 25 - DNA, 381 bp, Artificial Sequence
  FHF11-VH W36F (nt)
caggtgcagctgcagcagtctggaccaggactggtgaagcctagccagaccctgtctgtgacatgcggaatctccggcgacagcgtgtccagccactccgccgctttcaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 26 - protein, 127 aa, Artificial Sequence
  FHF11-VH W36F (aa)
QVQLQQSGPGLVKPSQTLSVTCGISGDSVSSHSAAFNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 27 - DNA, 381 bp, Artificial Sequence
  FHF11-VH W59F (nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcaagttctacaatgactatgccgtgtctgtgaagtccaggatcaccatcaacccagatacatccaagaatcagtttagcctgcagctgatctctgtgacccccgaggacacagccgtgtactattgtgccagagtgggcgctatgaccttcggcctgctgacaggcggaatggacgtgtggggacagggaaccacagtgacagtgtcttcc

SEQ ID NO: 28 - protein, 127 aa, Artificial Sequence
  FHF11-VH W59F (aa)
QVQLQQSGPGLVKPSQTLSVTCGISGDSVSSHSAAWNWIRQSPSRGLEWLGRTYYRSKFYNDYAVSVKSRITINPDTSKNQFSLQLISVTPEDTAVYYCARVGAMTFGLLTGGMDVWGQGTTVTVSS

SEQ ID NO: 29 - protein, 9 aa, Artificial Sequence
  FHF11-VH W59F CDRH2 (aa)
TYYRSKFYN

SEQ ID NO: 30 - DNA, 381 bp, Artificial Sequence
  FHF11v3 VH (nt)
caggtgcagctgcagcagtctggaccaggactggtgaagcctagccagaccctgtctgtgacatgcggcatctccggcgacagcgtgtccagctactccgccgcttggaactggatcagacagagcccatctaggggactggagtggctgggaaggacctactatcggagcaagtggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 31 - protein, 127 aa, Artificial Sequence
  FHF11v3 VH (aa)
QVQLQQSGPGLVKPSQTLSVTCGISGDSVSSYSAAWNWIRQSPSRGLEWLGRTYYRSKWYNDYAVSVKSRITINPDTSKNQFSLQLISVTPEDTAVYYCARVGAMTFGLLTGGMDVWGQGTTVTVSS

SEQ ID NO: 32 - protein, 10 aa, Artificial Sequence
  FHF11v3 CDRH1 (aa)
GDSVSSYSAA

SEQ ID NO: 33 - DNA, 381 bp, Artificial Sequence
  FHF11v6 VH (nt)
caggtgcagctgcagcagtctggaccaggactggtgaagcctagccagaccctgtctgtgacatgcggaatctccggcgacagcgtgtccagccactccgccgcttggaactggatcagacagagcccatctaggggactggagtggctgggaaggacctactatcggagcggctggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 34 - protein, 127 aa, Artificial Sequence
  FHF11v6 VH (aa)
QVQLQQSGPGLVKPSQTLSVTCGISGDSVSSHSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSS

SEQ ID NO: 35 - protein, 9 aa, Artificial Sequence
  FHF11v6 CDRH2 (aa)
TYYRSGWYN

SEQ ID NO: 36 - DNA, 381 bp, Artificial Sequence
  FHF11v9 VH (nt)
caggtgcagctgcagcagtctggaccaggactggtgaagcctagccagaccctgtctgtgacatgcggcatctccggcgacagcgtgtccagctactccgccgcttggaactggatcagacagagcccatctaggggactggagtggctgggaaggacctactatcggagcggctggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttcc

SEQ ID NO: 37 - protein, 127 aa, Artificial Sequence
  FHF11v9 VH (aa)
QVQLQQSGPGLVKPSQTLSVTCGISGDSVSSYSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSS

SEQ ID NO: 38 - DNA, 381 bp, Artificial Sequence
  FHF12-VH-W36F (nt)
caggtgcagctgcagcagtctggaccaggactggtgaagcctagccagaccctgtctgtgacatgcgctatctccggcgacagcgtgtccagccactccgccgctttcaactggatcagacagagcccatctaggggactggagtggctgggaaggacctactatcggagcaagtggtacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 39 - protein, 127 aa, Artificial Sequence
  FHF12-VH-W36F (aa)
QVQLQQSGPGLVKPSQTLSVTCAISGDSVSSHSAAFNWIRQSPSRGLEWLGRTYYRSKWYNDYAVSVKSRITINPDTSKNQFSLQLVSVTPEDTAVYYCARVGAATFGILTGGMDVWGQGTTVTVSS

SEQ ID NO: 40 - DNA, 381 bp, Artificial Sequence
  FHF12-VH-W59F (nt)
caggtgcagctgcagcagtctggaccaggactggtgaagcctagccagaccctgtctgtgacatgcgctatctccggcgacagcgtgtccagccactccgccgcttggaactggatcagacagagcccatctaggggactggagtggctgggaaggacctactatcggagcaagttctacaatgactatgccgtgtccgtgaagtccaggatcaccatcaacccagatacatccaagaatcagttcagcctgcagctggtgtctgtgacccccgaggacacagccgtgtactattgtgctagagtgggcgccgctacctttggcatcctgacaggcggaatggacgtgtggggacagggaaccacagtgacagtgtcttcc

SEQ ID NO: 41 - protein, 127 aa, Artificial Sequence
  FHF12-VH-W59F (aa)
QVQLQQSGPGLVKPSQTLSVTCAISGDSVSSHSAAWNWIRQSPSRGLEWLGRTYYRSKFYNDYAVSVKSRITINPDTSKNQFSLQLVSVTPEDTAVYYCARVGAATFGILTGGMDVWGQGTTVTVSS

SEQ ID NO: 42 - protein, 9 aa, Artificial Sequence
  FHF12-CDRH2-W59F (aa)
TYYRSKFYN

SEQ ID NO: 43 - protein, 128 aa, Artificial Sequence
  FM08 VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 44 - protein, 103 aa, Artificial Sequence
  Synthetic sequence FM08 VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 45 - protein, 217 aa, Artificial Sequence
  WT hIgG1 Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 19 aa, Artificial Sequence
  Chimeric hinge sequence
ESKYGPPCPPCPAPPVAGP

SEQ ID NO: 47 - protein, 330 aa, Artificial Sequence
  Synthetic sequence IgHG1*01, G1m3 CH1-CH3 with M428L and N434S mutations and C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 48 - protein, 107 aa, Artificial Sequence [sequence omitted]

SEQ ID NO: 49 - protein, 329 aa, Artificial Sequence
  Synthetic sequence IgHG1*01, G1m3 CH1-CH3 with M428L and N434S mutations and without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 50 - protein, 457 aa, Artificial Sequence
  Synthetic sequence FHF11v9-MLNS heavy chain with C-terminal lysine
QVQLQQSGPGLVKPSQTLSVTCGISGDSVSSYSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 51 - protein, 456 aa, Artificial Sequence
  Synthetic sequence FHF11v9-MLNS heavy chain without C-terminal lysine
QVQLQQSGPGLVKPSQTLSVTCGISGDSVSSYSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 52 - protein, 216 aa, Artificial Sequence
  Synthetic sequence FHF11v9-MLNS light chain
EIVLTQSPGTQSLSPGERATLSCRASQSLSRSYLAWYQQRPGKPPRLLIYGASSRATGIPDRFSGSGSGTDFSLTISSLEPEDSAMYFCQYYGDSPLFSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 11 aa, Artificial Sequence
  Synthetic sequence - FHF11 and FHF12 CDRH1 with flanking Phe
GDSVSSHSAAF

SEQ ID NO: 54 - DNA, 1371 bp, Artificial Sequence
  Synthetic sequence - FHF11v9 heavy chain nucleotide sequence
caggtgcagctgcagcagtctggaccaggactggtgaagcctagccagaccctgtctgtgacatgcggcatctccggcgacagcgtgtccagctactccgccgcttggaactggatcagacagagcccatctaggggactggagtggctgggaaggacctactatcggagcggctggtacaatgactatgccgtgtctgtgaagtccaggatcaccatcaacccagatacatccaagaatcagttcagcctgcagctgatctctgtgacccccgaggacacagccgtgtactattgtgccagagtgggcgctatgacctttggcctgctgacaggcggaatggacgtgtggggacagggaaccacagtgacagtgtcttccgcatcgaccaagggcccatcggtcttccccctggcaccaagtagcaagagcacatccggtggcacagccgccctgggttgtctggtgaaagattatttccctgagcccgtgacagtctcctggaactctggcgccctgacctccggagtgcacacattccctgctgtgctgcagtccagcggcctgtactccctgtcttccgtggtgaccgtgccaagctcttccctgggcacccagacatatatctgcaacgtgaatcacaagccttccaatacaaaggtggacaagagggtggagccaaagagctgtgataagacccatacatgcccaccttgtccagctccagagctgctgggcggcccatccgtgttcctgtttccacccaagcccaaggacaccctgatgatctctagaaccccagaggtgacatgcgtggtggtggacgtgtcccacgaggatcccgaggtgaagtttaactggtacgtggatggcgtggaggtgcataatgctaagacaaagcccagggaggagcagtacaacagcacctatcgggtggtgtctgtgctgacagtgctgcatcaggactggctgaacggcaaggagtataagtgcaaggtgagcaataaggccctgcctgctccaatcgagaagaccatctctaaggccaagggccagcccagagagcctcaggtgtacacactgcctccaagccgcgaggagatgaccaagaaccaggtgtctctgacatgtctggtgaagggcttctatccctctgacatcgctgtggagtgggagtccaatggccagcctgagaacaattacaagaccacaccccctgtgctggactccgatggcagcttctttctgtattccaagctgaccgtggataagagcaggtggcagcagggcaacgtgttctcctgttctgtgatgcacgaagccctgcacaaccattatactcagaagtccctgtccctgtcccctggaaaa

SEQ ID NO: 55 - DNA, 649 bp, Artificial Sequence
  Synthetic sequence - FHF11v9 light chain nucleotide sequence
gaaattgtgttgacgcagtctccaggcacccagtctttgtctccaggggaaagagccaccctctcctgcagggccagtcagagtctgagccgcagctacttagcctggtaccagcagagacctggcaagcctcccaggctcctcatctatggtgcatccagcagggccactggcatcccagacaggttcagtggcagtgggtctgggacagacttcagtctcaccatcagcagtctggagcctgaagattctgcaatgtatttctgtcagtactatggtgattcacctctattcagtttcggcccagggaccaaagtggatatcaaaccgtacggtggctgcaccatctgtcttcatcttcccgccatctgatgagcaattgaaatctggaactgcctccgtggtgtgcctgctgaacaatttctaccccagggaggccaaggtgcagtggaaggtggacaacgctctgcagagcggcaattctcaggagtccgtgaccgagcaggacagcaaggattctacatattccctgtccagcaccctgacactgagcaaggccgattacgagaagcacaaggtgtatgcttgtgaggtgacccatcagggcctgtcttcccctgtgacaaagtctttcaacaggggagagtgt

SEQ ID NO: 56 - protein, 457 aa, Artificial Sequence
  Synthetic sequence - FHF11v9 heavy chain
QVQLQQSGPGLVKPSQTLSVTCGISGDSVSSYSAAWNWIRQSPSRGLEWLGRTYYRSGWYNDYAVSVKSRITINPDTSKNQFSLQLISVTPEDTAVYYCARVGAMTFGLLTGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 127 aa, Artificial Sequence
  Synthetic sequence - FHF-VH-UCA
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARVGAMIFGLLTGGMDVWGQGTTVTVSS

SEQ ID NO: 58 - protein, 109 aa, Artificial Sequence
  Synthetic sequence - FHF-VL-UCA
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLFTFGPGTKVDIK

(19) United States
(12) Patent Application Publication
(10) Pub. No.: US 2024/0317841 A1
(43) Pub. Date: Sep. 26, 2024

(54) ANTIBODIES AGAINST INFLUENZA A VIRUSES

(71) Applicants: Vir Biotechnology, Inc., San Francisco, CA (US); Humabs BioMed SA, Bellinzona (CH)

(72) Inventors: Davide CORTI, Bellinzona (CH); Matteo Samuele PIZZUTO, Bellinzona (CH); Fabrizia ZATTA, Bellinzona (CH); Elisabetta CAMERONI, Bellinzona (CH); Gyorgy SNELL, San Francisco, CA (US)

(21) Appl. No.: 18/253,383

(22) PCT Filed: Nov. 19, 2021
(86) PCT No.: PCT/US2021/060123

§ 371 Date: May 17, 2023

Related U.S. Application Data
(60) Provisional application No. 63/117,437, filed on Nov. 23, 2020.
(60) Provisional application No. 63/123,419, filed on Dec. 9, 2020.

(51) Int. Cl. C07K 16/10; A61K 39/00; A61P 31/16

(52) U.S. Cl. CPC C07K 16/1018; A61P 31/16; A61K 2039/505; C07K 2317/33; C07K 2317/52; C07K 2317/565; C07K 2317/72; C07K 2317/76; C07K 2317/92

(57) ABSTRACT

The instant disclosure provides antibodies and antigen-binding fragments thereof that can bind to an influenza A virus hemagglutinin (HA) and can neutralize a IAV infection. Also provided are polynucleotides that encode an antibody or antigen-binding fragment, vectors that comprise such polynucleotides, host cells that can express the antibodies or antigen-binding fragments, related compositions, and methods of using the herein disclosed compositions to, for example, treat or prevent an IAV infection.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_41 iWO_SEQUENCE_LISTING.txt. The text file is 44.4 KB, was created on Nov. 16, 2021, and is being submitted electronically via EFS-Web.

### BACKGROUND

Influenza is an infectious disease which spreads around the world in yearly outbreaks, resulting per year in about three million to about five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually symptoms begin about two days after exposure to the virus. Complications of influenza may include pneumonia, sinus infections, and worsening of previous health problems such as asthma or heart failure, sepsis or exacerbation of chronic underlying disease.

Influenza is caused by influenza virus, an antigenically and genetically diverse group of viruses of the family Orthomyxoviridae that contain a negative-sense, single-stranded, segmented RNA genome. Of the four types of influenza virus (A, B, C and D), three types (A, B and C) are known to affect humans. Influenza type A viruses are typically the most virulent human pathogens and cause the most severe disease.

Influenza A viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 includes H1, H2, H5, H6, H8, H9, H11, H12, H13, H16 and H17 subtypes, and group 2 includes H3, H4, H7, H10, H14 and H15 subtypes. While all subtypes are found in birds, mostly H1, H2 and H3 subtypes cause disease in humans. H5, H7 and H9 subtypes have caused sporadic severe infections in humans and may generate a new pandemic. Influenza A viruses continuously evolve generating new variants, a phenomenon called antigenic drift. As a consequence, antibodies produced in response to past viruses may be poorly- or non-protective against new drifted viruses. A consequence is that new vaccines have to be produced every year against H1 and H3 viruses that are predicted to emerge, a process that is very costly, and not always efficient. The same applies to the production of a H5 influenza vaccine.

HA is a major surface protein of influenza A virus, and is the primary target of neutralizing antibodies that are induced by infection or vaccination. Without wishing to be bound by theory, HA is responsible for binding the virus to cells with sialic acid on the cell membrane, such as cells in the upper respiratory tract or erythrocytes. In addition, HA mediates fusion of the viral envelope to the endosome membrane, following a reduction in pH, facilitating escape of the virus into the cytoplasm.

HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha helical coiled-coil structure and primarily forms the “stem” or “stalk” region of HA, while the HA1 region is a small globular domain containing a mix of a/P structures (“head” region of HA). The globular HA head region mediates binding to the sialic acid receptor, while the HA stem mediates the subsequent fusion between the viral and cellular membranes that is triggered in endosome by low pH. While the immunodominant HA globular head domain has high plasticity with distinct antigenic sites undergoing consistent antigenic drift, the HA stem region is relatively conserved among subtypes. Current influenza vaccines mostly induce an immune response against the immunodominant and variable HA head region, which evolves faster than the stem region of HA (Kirkpatrick E, Qiu X, Wilson P C, Bahl J, Krammer F. The influenza virus hemagglutinin head evolves faster than the stalk domain. Sci Rep. 2018 Jul. 11; 8(1):10432). Therefore, a particular influenza vaccine usually confers protection for no more than a few years and annual re-development of influenza vaccines is required. Accordingly, modalities for broadly neutralizing influenza A virus infections, in particular with improved potency, are needed.

### BRIEF DESCRIPTION OF THE DRAWINGS

FIG. 1 shows a workflow for anti-“HA” (hemagglutinin) stem monoclonal antibody discovery, described in further detail in Example 1.

FIG. 2 shows binding of monoclonal antibodies “FHF11” (also referred-to herein as “FHF11-WT”; VH: SEQ ID NO.:2; VL: SEQ ID NO.:8) and “FHF12” (VH: SEQ ID NO.:14; VL: SEQ ID NO.:20) to influenza A virus (IAV)-derived hemagglutinins (HA)s, as determined by flow cytometry using HA-expressing mammalian target cells. A comparator antibody “FM08” (VH: SEQ ID NO.:43; VL: SEQ ID NO.:44) was also tested.

FIGS. 3A and 3B show binding of FHF11 and FHF12 to group I IAV-derived H1, H2, H5, and H9 (FIG. 3A) and group II IAV-derived H3 (FIG. 3B) measured by ELISA, reported as Log EC50 (ng/ml). Binding by a comparator antibody, FM08, was also measured.

FIG. 4 shows binding of FHF11 and FHF12 to HA from an H1N1 Swine Eurasian avian-like (EA) strain, A/Swine/Jiangsu/J004/2018 expressed on mammalian cells, was measured by flow cytometry. Binding was measured at antibody concentrations of 50 μg/ml, 10 μg/ml, 2 μg/ml, and 0.4 μg/ml. Mock staining is shown as a negative control. Binding by a comparator antibody, FM08, was also measured.

FIG. 5 shows lack of polyreactivity of FHF11 and FHF12, as tested against human epithelial type 2 (HEP-2) cells. A polyreactive comparator, “FI6v3.11.18”, was included as a positive control, and anti-paramyxovirus antibody “MPE8” was included as a negative control.

FIGS. 6A and 6B show in vitro neutralization of H1N1 and H3N2 IAV pseudovirus by FHF11 and FHF12. FIG. 6A shows neutralization of H1N1 A/California//07/2009. FIG. 6B shows neutralization of H3N2 A/Aichi/2/68. Data for comparator antibodies FM08 and FY1 is also shown.

FIGS. 7A and 7B show in vitro neutralization of H5 and H7 pseudotyped viruses by FHF11 and FHF12. Data for comparator antibody FMO8 is also shown.

FIG. 7A shows neutralization of H5/VN/11/94 pp. FIG. 7B shows neutralization of H7/IT/99 pp.

FIGS. 8A and 8B show activation of (F158) FcγRIIIa (FIG. 8A) and (V158) FcγRIIa (FIG. 8B) variants by FHF11, as described in Example 1. FMO8 (comprising M428L and N434S (“LS”, also identified as “MLNS” herein) mutations in the Fc), and FY1 (comprising G236R and L328R (“GRLR”) mutations in the Fc) were included as comparators.

FIGS. 9A-9D provide schematic illustrations of light chain and heavy chain complementarity determining regions (CDRs) of FMO8 (which utilizes the same VH6-1/DH3-3 genes as FHF11 and FHF12) interacting with HA. Interactions of FMO8 CDRs with the influenza HA (FIG. 9A), with HA fusion peptide (FIG. 9B), with a hydrophobic groove in HA (FIG. 9C), and HA Helix A (FIG. 9D) are illustrated.

FIGS. 10A-10B relate to FHF11 and sequence-engineered variants thereof. FIG. 10A summarizes binding of FHF11-WT and fifteen (15) FHF11 variant antibodies (v1 to v15) to mammalian cells expressing different HA subtypes derived from viruses circulating in the animal reservoir, as measured by FACS. Data for comparator antibody FMO8 is also shown. Staining with secondary antibody only and full staining of mock-infected cells were included as negative controls. FIG. 10B summarizes mutations in the variable region(s) (versus parental FHF11-WT) that were made to produce the indicated FHF11 variants.

FIG. 11 shows binding (reported as LogEC50 (ng/mL)), by FHF11-WT (VH: SEQ ID NO.: 2; VL: SEQ ID NO.:8), FHF11v3 (VH: SEQ ID NO.:31; VL: SEQ ID NO.:8), FHF11v6 (VH: SEQ ID NO.:34; VL: SEQ ID NO.: 8), and FHF11v9 (VH: SEQ ID NO.:37; VL: SEQ ID NO.:8), as well as by comparator antibodies FYi and FMO8, to HAs derived from a panel of human H3N2 IAV subtypes, as measured by ELISA. The panel is shown to the right of the graph. Geometric mean EC50 and geometric mean SD factor EC50 for each antibody are shown in the table at the bottom right of the figure.

FIG. 12 shows binding (reported as LogEC50 (ng/mL)) by FHF11-WT, FHF11v3, FHF11v6, and FHF11v9, as well as by comparator antibodies FYi and FM08, to HAs derived from a panel of human H1N1, H2N2, H5N1, and H9N2 IAV subtypes. The panel is shown to the right of the graph. Geometric mean EC50 and Geometric mean SD factor EC50 for each antibody are shown in the table at the bottom right of the figure.

FIG. 13 shows binding kinetics of FHF11-WT, FHF11v3, FHF11v6, and FHFI1v9, as well as of comparator antibodies FYi and FM08, to H5 HA (“HA-5”), as measured by Bio-Layer Interferometry (BLI). Dissociation is reported as kdis (1/s), association is reported as kon (1/Ms), and KD was calculated from the ratio of kdis/kon.

FIG. 14 shows binding kinetics of FHF11-WT, FHF11v3, FHF11v6, and FHF11v9, as well as of comparator antibodies FYi and FM08, to H7 HA (“HA-7”) as measured by BLL. Dissociation is reported as kdis (1/s), association is reported as kon (1/Ms), and KD was calculated from the ratio of kdis/kon.

FIG. 15A shows in vitro neutralization of H5 pseudovirus by FHF11 (“FHF11 WT” in the figure) and fifteen (15) variant antibodies generated from FHF11 WT, at increasing antibody concentrations (FIG. 15A). FIG. 15B shows in vitro neutralization of H5 pseudovirus by FHF11 (“FHF11 WT” in the figure) and twelve (12) variant antibodies generated from FHF11 WT and reported as IC50 (ng/ml) values.

FIG. 15C shows neutralization data for FHF11-WT and three variant antibodies that were selected for further analysis, FHF11v3, FHF11v6, and FHF11v9. Calculated IC50 values (ng/mL) are shown at the right of FIGS. 15A and 15C.

FIGS. 16A-16F show in vitro neutralization of H1N1 and H3N2 subtypes H1N1 A/PR/8/34 (FIG. 16A), H1N1 A/Solomon Islands/3/06 (FIG. 16B), H1N1 A/California/2009 (FIG. 16C), H3N2 A/Aichi/2/68 (FIG. 16D), H3N2 A/Brisbane/10/07 (FIG. 16E), and H3N2 A/Hong Kong/68 (FIG. 16F) by FHF11-WT and variant antibodies FHF11v3, FHF11v6, and FHF11v9. Data for comparator antibodies FYi and FM08 is also shown. Calculated IC50 and IC90 values (ng/mL) are shown below the graph in each figure.

FIGS. 17A and 17B show activation of FcγRIIIa by FHF11v9. Activation was measured using an NFAT-mediated luciferase reporter in engineered Jurkat cells following contact with influenza-infected A549 cells. A549 cells were pre-infected with H1N1 (FIG. 17A) or H3N2 (FIG. 17B). Data for comparator antibodies FM08_LS and FYl-GRLR is also shown.

FIGS. 18A and 18B show activation of FcγRIIa by FHF11v9. Activation was measured using an NFAT-mediated luciferase reporter in engineered Jurkat cells following contact with influenza-infected A549 cells. A549 cells were pre-infected with H1N1 (FIG. 18A) or H3N2 (FIG. 18B). Data for comparator antibodies FM08_LS and FYl-GRLR is also shown.

FIG. 19 shows pharmacokinetic properties of FHF11v9 (“FHF11v9-LS”), FHF12 (“FHF12-LS”), and FM08 (“FM08_LS”) M428L/N434S Fc variants in tg32 mice. Antibody was administered at the indicated dose. Calculated half-life values are identified by the boxes.

FIGS. 20A-20D show measurements of body weight over fifteen days in BALB/c mice infected with H1N1 A/Puerto Rico/8/34 following pre-treatment with 15 FHF11v9. Antibody was administered at 6 mg/kg (FIG. 20A), 2 mg/kg (FIG. 20B), 0.6 mg/kg (FIG. 20C), or 0.2 mg/kg (FIG. 20D), one day prior to infection with a LD90 (90% lethal dose) of A/Puerto Rico/8/34. Body weight of mice receiving a vehicle control was also measured (left graph in each figure).

FIGS. 21A-21D show measurements of body weight over fifteen days in BALB/c mice infected with H3N2 A/Hong Kong/68 following pre-treatment with FHF11v9. Antibody was administered at 6 mg/kg (FIG. 21A), 2 mg/kg (FIG. 21B), 0.6 mg/kg (FIG. 21C), or 0.2 mg/kg (FIG. 21D), one day prior to infection with a LD90 (90% lethal dose) of H3N2 A/Hong Kong/68. Body weight of mice receiving a vehicle control was also measured (left graph in each figure).

FIGS. 22A and 22B show survival over fifteen days of BALB/c mice infected with H1N1 A/Puerto Rico/8/34 (FIG. 22A) or H3N2 A/Hong Kong/8/68 (FIG. 22B) following pre-treatment with FHIF11v9 at the indicated dose. Survival in mice pre-treated with a vehicle control was also measured.

FIG. 23 shows in vitro neutralization of H1N1 and H3N2 subtypes by FHF11v9 and comparator antibody FM08_MLNS (aka FM08_LS), measured by IAV nucleoprotein staining.

FIGS. 24A and 24B show the design of an in vivo study to evaluate prophylactic activity of FHF11v9 (“mAb-11” in FIG. 24A) and a comparator antibody, FM08_MLNS (“mAb-08” in FIG. 24A), in Balb/c mice infected with A/Puerto Rico/8/34 or A/Hong Kong/8/68. FIG. 24A shows inter alia the dosing and virus strains used in the study. FIG. 24B shows the timeline and endpoints of the study.

FIG. 25 shows negative area-under-the-curve peak values (reported as EC50 in μg/ml) compared with IgG concentration in serum from area-under-the-curve analysis of body weight loss in BALB/c mice infected with A/Puerto Rico/8/34 (H1N1, left graph) or A/Hong Kong/8/68 (H3N2, right graph) following treatment with FHF11v9 or comparator antibody FM08_MLNS.

FIGS. 26A and 26B show in vivo pharmacokinetic properties of FHF11v9 and comparator antibody FM08_MLNS in SCID tg32 mice. FIG. 26A shows concentration of antibody over time (reported as μg/ml) over 30 days post-administration. The table in FIG. 26B shows calculated half-life (reported in days) highlighted by a box.

### DETAILED DESCRIPTION

Provided herein are antibodies and antigen-binding fragments that can bind to and potently neutralize infection by various influenza A viruses (IAVs). Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) a IAV infection in a subject and/or in the manufacture of a medicament for treating a IAV infection in a subject.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. In some embodiments, “about” includes 20%, +15%, +10%, or +5% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA, also referred to as deoxyribonucleic acid), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine. These features are known in the art and are discussed in, for example, Zhang et al. Front. Immunol., DOI=10.3389/fimmu.2019.00594 (2019); Eyler et al. PNAS 116(46): 23068-23071; DOI: 10.1073/pnas.1821754116 (2019); Nance and Meier, ACS Cent. Sci. 2021, 7, 5, 15 748-756; doi.org/10.1021/acscentsci.lc00197 (2021), and van Hoecke and Roose, J. Translational Med 17:54 (2019); https://doi.org/10.1186/s12967-019-1804-8, which modified nucleosides and mRNA features are incorporated herein by reference. Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise.

Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mites et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a 7-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a IAV infection, a “host” refers to a cell or a subject infected with the IAV.

“Antigen” or “Ag”, as used herein, refers to an immunogenic molecule that provokes an immune response. This immune response may involve antibody production, activation of specific immunologically-competent cells, activation of complement, antibody dependent cytotoxicicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a IAV HA, such as present in a virion, or expressed or presented on the surface of a cell infected by the IAV.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

In one aspect, the present disclosure provides an isolated antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a IAV HA.

In certain embodiments, an antibody or antigen-binding fragment of the present disclosure associates with or unites with a HA while not significantly associating or uniting with any other molecules or components in a sample.

In certain embodiments, an antibody or antigen-binding fragment of the present disclosure specifically binds to a IAV HA. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M1, at least 10⁹ M1, at least 10¹⁰ M⁻¹, at least 10¹¹ M⁻¹, at least 10¹² M-1, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, biolayer interferometry and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a IAV HA antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo software (TreeStar). In some embodiments, positive binding can be defined by differential staining by antibody of IAV HA-expressing cells versus control (e.g., mock) cells.

In some embodiments an antibody or antigen-binding fragment of the present disclosure binds to a HA protein, as measured using biolayer interferometry, or by surface plasmon resonance.

Certain characteristics of presently disclosed antibodies or antigen-binding fragments may be described using IC50 or EC50 values. In certain embodiments, the IC50 is the concentration of a composition (e.g., antibody) that results in half-maximal inhibition of the indicated biological or biochemical function, activity, or response. In certain embodiments, the EC50 is the concentration of a composition that provides the half-maximal response in the assay. In some embodiments, e.g., for describing the ability of a presently disclosed antibody or antigen-binding fragment to neutralize infection by IAV, IC50 and EC50 are used interchangeably.

In certain embodiments, an antibody of the present disclosure is capable of neutralizing infection by IAV. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment can be capable of preventing and/or neutralizing a IAV infection in an in vitro model of infection and/or in an in vivo animal model of infection and/or in a human.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “V_{L}” or “VL” and “V_{H}” or “VH” refer to the variable binding region from an antibody light chain and an antibody heavy chain, respectively. In certain embodiments, a VL is a kappa (x) class (also “VK” herein). In certain embodiments, a VL is a lambda (k) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, Contact, North, Martin, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001); North et al. J Mol Biol. (2011) 406:228-56; doi:10.1016/j.jmb.2010.10.030; Abhinandan and Martin, Mol Immunol. (2008) 45:3832-9.10.1016/j.molimm.2008.05.022). The antibody and CDR numbering systems of these references are incorporated herein by reference. Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

In certain embodiments, an antibody or antigen-binding fragment is provided that comprises CDRs of a VH sequence according to any one of SEQ ID NOs.: 2, 26, 28, 31, 34, 37, 14, 39 and 41, and in a VL sequence according to any one of SEQ ID NOs.: 8 or 20, in accordance with any known CDR numbering method, including the Kabat, Chothia, EU, IMGT, Martin (Enhanced Chothia), Contact, North, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software (www.chemcomp.com). In certain embodiments, CDRs are according to the Kabat numbering method.

In some embodiments, CDRs are according to the IMGT numbering method. In certain embodiments, the present disclosure provides an antibody, or antigen-binding fragment thereof, comprising a heavy chain variable domain (VH) comprising a complementarity determining region (CDR)H1, a CDRH2, and a CDRH3, and a light chain variable domain (VL) comprising a CDRL1, a CDRL2, and a CDRL3: (i) the CDRH1 comprises or consists of the amino acid sequence of any one of SEQ ID NOs.: 3, 32, or 15, or a functional variant thereof comprising one, two, or three acid substitutions, one or more of which substitutions is optionally a conservative substitution and/or is a substitution to a germline-encoded amino acid; and/or (ii) the CDRH2 comprises or consists of the amino acid sequence of any one of SEQ ID NOs.: 4, 29, 35, 16, or 42, or a functional variant thereof comprising one, two, or three amino acid substitutions, one or more of which substitutions is optionally a conservative substitution and/or is a substitution to a germline-encoded amino acid; and/or (iii) the CDRH3 comprises or consists of the amino acid sequence of any one of SEQ ID NOs.: 5 or 17, or a functional variant thereof comprising one, two, or three amino acid substitutions, one or more of which substitutions is optionally a conservative substitution and/or is a substitution to a germline-encoded amino acid; and/or (iv) the CDRL1 comprises or consists of the amino acid sequence of any one of SEQ ID NOs.: 9 or 21, or a functional variant thereof comprising one, two, or three amino acid substitutions, one or more of which substitutions is optionally a conservative substitution and/or is a substitution to a germline-encoded amino acid; and/or (v) the CDRL2 optionally comprises or consists of the amino acid sequence of any one of SEQ ID NOs.: 10 or 22, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence of any one of SEQ ID NOs.: 11 or 23, or a functional variant thereof comprising having one, two, or three amino acid substitutions, one or more of which substitutions is optionally a conservative substitution and/or is a substitution to a germline-encoded amino acid, wherein the antibody or antigen-binding fragment is capable of binding to an influenza A virus (IAV) hemagglutinin (HA); e.g. when the IAV HA is expressed on a cell surface of a host cell and/or on a virion.

In some embodiments, the antibody or an antigen-binding fragment is capable of neutralizing an IAV infection in an in vitro model of infection and/or in an in vivo animal model of infection and/or in a human, wherein, optionally, the in vitro model of infection comprises a target cell and a pseudovirus or a target cell and a live virus.

In certain embodiments, an antibody or an antigen-binding fragment of the present disclosure comprises a CDRH1, a CDRH2, a CDRH3, a CDRL1, a CDRL2, and a CDRL3, wherein each CDR is independently selected from a corresponding CDR of an HA-specific antibody as provided in Table 1 and/or Table 2. That is, all combinations of CDRs from HA-specific antibodies provided in Table 1 and/or Table 2 are contemplated.

In certain embodiments, the antibody or an antigen-binding fragment comprises a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, CDRH3, CDRL1, CDRL2, and CDRL3 comprise or consist of the amino acid sequences of: (i) 3-5 and 9-11, respectively; (ii) 3, 29, 5 and 9-11, respectively; (iii) 32, 4, 5 and 9-11, respectively; (iv) 3, 35, 5 and 9-11, respectively; (v) 32, 35, 5, and 9-11, respectively; (vi) 15-17 and 21-23, respectively; or (vii) 15, 42, 17 and 21-23, respectively. In certain embodiments, the antibody or an antigen-binding fragment comprises a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, CDRH3, CDRL1, CDRL2, and CDRL3 comprise or consist of the amino acid sequences of: (i) 3, 29, 5 and 9-11, respectively; (ii) 3, 35, 17 and 9-11, respectively; or (iii) 32, 35, 17, and 9-11, respectively.

In some embodiments, an antibody, or antigen-binding fragment thereof, is provided that comprises: (1) a heavy chain variable domain (VH) comprising the amino acid sequence of SEQ ID NO.:53, the amino acid sequence of any one of SEQ ID NOs.:4, 29, and 35, and the amino acid sequence of any one of SEQ ID NOs.:5 and 17; and (2) a light chain variable domain (VL) comprising the amino acid sequences of SEQ ID NOs.:9-11, wherein the antibody or antigen-binding fragment is capable of binding to an influenza A virus (IAV) hemagglutinin (HA).

In some embodiments, an antibody, or antigen-binding fragment thereof, is provided that comprises a heavy chain variable domain (VH) and a light chain variable domain (VL), wherein: (i) the VH comprises a CDRH1, a CDRH2, and a CDRH3 according to the VH amino acid sequence set forth in any one of SEQ ID NOs.: 37, 2, 26, 28, 31, 34, 14, 39 and 41; and (ii) the VL comprises a CDRL1, a CDRL2, and a CDRL3 according to the VL amino acid sequence set forth in SEQ ID NO.:2, wherein the antibody or antigen-binding fragment is capable of binding to an influenza A virus (IAV) hemagglutinin (HA).

In some embodiments, an antibody, or antigen-binding fragment thereof, is provided that comprises a heavy chain variable domain (VH) and a light chain variable domain (VL), wherein: (i) the VH comprises a CDRH1, a CDRH2, and a CDRH3 according to the VH amino acid sequence set forth in any one of SEQ ID NOs.: 37, 2, 26, 28, 31, 34, 14, 39 and 41; and (ii) the VL comprises a CDRL1, a CDRL2, and a CDRL3 according to the VL amino acid sequence set forth in SEQ ID NO.:8, wherein the antibody or antigen-binding fragment is capable of binding to an influenza A virus (IAV) hemagglutinin (HA). In further embodiments, the CDRs are according to the IMGT, Kabat, Chothia, AhHo, or North numbering system.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In any of the presently disclosed embodiments, an antibody or antigen-binding fragment of the present disclosure may comprise any one or more of CL, a CH1, a CH2, and a CH3. In certain embodiments, a CL comprises an amino acid sequence having 90%, 91%, 92%, 93%, 94%, 95%, 96%, 975, 98%, 99%, or 100% identity to the amino acid sequence of SEQ ID NO.:48. In certain embodiments, a CH1-CH2-CH3 comprises an amino acid sequence having 90%, 91%, 92%, 93%, 94%, 95%, 96%, 975, 98%, 99%, or 100% identity to the amino acid sequence of SEQ ID NO.:47 or SEQ ID NO.:49. It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Both the Fab and F(ab′)₂ are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. In some embodiments, the scFv polypeptide comprises a polypeptide linker disposed between and linking the VH and VL domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (Bird et al., Science 242:423-426 (1988)) and the pentamer Gly-Gly-Gly-Gly-Ser when present in a single iteration or repeated 1 to 5 or more times, or more. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

scFvs can be constructed using any combination of the VH and VL sequences or any combination of the CDRH1, CDRH2, CDRH3, CDRL1, CDRL2, and CDRL3 sequences disclosed herein.

In some embodiments, linker sequences are not required; for example, when the first and second polypeptides have non-essential N-terminal amino acid regions that can be used to separate the functional domains and prevent steric interference.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a IAV HA antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations. Variable region and CDR amino acid sequences of exemplary IAV HA antibodies of the present disclosure are provided in Table 1 herein.

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations in a variable region (e.g., VH, VL, framework or CDR) as compared to a presently disclosed (“parent”) antibody, wherein the variant antibody is capable of binding to a IAV HA antigen.

In certain embodiments, (i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 2, 26, 28, 31, 34, 37, 14, 39 and 41, wherein sequence variation with reference to SEQ ID NO.: 2, 26, 28, 31, 34, 37, 14, 39 or 41, respectively, is optionally comprised in one or more framework region and/or sequence variation comprises one or more substitution to a germline-encoded amino acid; and/or (ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 8 or 20, wherein sequence variation with respect to SEQ ID NO.:8 or 20, respectively, is optionally comprised in one or more framework regions and/or sequence variation comprises one or more substitution to a germline-encoded amino acid. In some embodiments, (i) the VH comprises or consists of an amino acid sequence having at least 80% identity to the amino acid sequence of any one of SEQ ID NOs.: 37, 2, 26, 28, 31, 34, 14, 39 and 41, and the VL comprises or consists of an amino acid sequence having at least 80% identity to the amino acid sequence of SEQ ID NO.:8; or (ii) the VH comprises or consists of an amino acid sequence having at least 80% identity to the amino acid sequence of any one of SEQ ID NOs.: 37, 2, 26, 28, 31, 34, 14, 39 and 41, and the VL comprises or consists of an amino acid sequence having at least 80% identity to the amino acid sequence of SEQ ID NO.:20.

In some embodiments, the VH and the VL comprise or consist of amino acid sequences having at least 80% identity to the amino acid sequences according to SEQ ID NOs.: (i) 2 and 8, respectively; (ii) 26 and 8, respectively; (iii) 28 and 8, respectively; (iv) 31 and 8, respectively; (v) 34 and 8, respectively; (vi) 37 and 8, respectively; (vii) 14 and 20, respectively; (viii) 39 and 20, respectively; or (ix) 41 and 20, respectively; or (x) 57 and 58, respectively. In other embodiments, the VH and the VL comprise or consist of amino acid sequences having at least 80% identity to to SEQ ID NOs.: (i) 2 and 20, respectively; (ii) 26 and 20, respectively; (iii) 28 and 20, respectively; (iv) 31 and 20, respectively; (v) 34 and 20, respectively; (vi) 37 and 20, respectively; (v) 14 and 8, respectively; (vi) 39 and 8, respectively; or (vii) 41 and 8, respectively.

In some embodiments, the VH is encoded by or derived from VH6-1, DH3-3, and JH6, and/or the VL is encoded by or derived from VK3-20 and JK3.

In certain embodiments, the VH comprises or consists of any VH amino acid sequence set forth in Table 1 and/or Table 2, and the VL comprises or consists of any VL amino acid sequence set forth in Table 1 and/or Table 2. In some embodiments, the VH and the VL comprise or consist of the amino acid sequences according to SEQ ID NOs.: (i) 2 and 8, respectively; (ii) 26 and 8, respectively; (iii) 28 and 8, respectively; (iv) 31 and 8, respectively; (v) 34 and 8, respectively; (vi) 37 and 8, respectively; (vii) 14 and 20, respectively; (viii) 39 and 20, respectively; or (ix) 41 and 20, respectively. In other embodiments, the VH and the VL comprise or consist of the amino acid sequences according to SEQ ID NOs.: (i) 2 and 20, respectively; (ii) 26 and 20, respectively; (iii) 28 and 20, respectively; (iv) 31 and 20, respectively; (v) 34 and 20, respectively; (vi) 37 and 20, respectively; (v) 14 and 8, respectively; (vi) 39 and 8, respectively; (vii) 41 and 8, respectively; or (viii) 57 and 58, respectively. In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)₂-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, k-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different eptiopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure constructed comprise any combination of the VH and VL sequences and/or any combination of the CDRH1, CDRH2, CDRH3, CDRL1, CDRL2, and CDRL3 sequences disclosed herein. A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different HA epitope, and a bispecific or multispecific antibody or antigen-binding fragment as provided herein can, in some embodiments, comprise a further HA-specific binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises a Fc polypeptide, or a fragment thereof. The “Fc” comprises the carboxy-terminal portions (i.e., the CH2 and CH3 domains of IgG) of both antibody H chains held together by disulfides. An Fc may comprise a dimer comprised of two Fc polypeptides (i.e., two CH2-CH3 polypeptides). Antibody “effector functions” refer to those biological activities attributable to the Fc region (a native sequence Fc region or amino acid sequence variant Fc region) of an antibody, and vary with the antibody isotype.

Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/1332E, P2571/Q311, K326W/E333S, S239D/1332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the Clq protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to Clq. The role of Glu318, Lys320 and Lys 322 residues in the binding of Clq was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRII1B and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIII1B, which is highly expressed on neutrophils.

FcγRIIA is found on many cells involved in killing (e.g. macrophages, monocytes, neutrophils) and seems able to activate the killing process. FcγRII1B seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with higher affinity, and the “R131” variant, which binds to IgG1 Fc with lower affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with lower affinity, and the “V158” variant, which binds to IgG1 Fc with higher affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment thereof comprises S at position 239 (EU numbering).

In certain embodiments, the Fc polypeptide or fragment thereof may comprise or consist of at least a portion of an Fc polypeptide or fragment thereof that is involved in FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fe and a half-life-extending mutation comprises any one or more of: M428L; N434S; N434H; N434A; N434S; M252Y; S254T; T256E; T250Q; P2571 Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A. In some embodiments, an antibody or antigen-binding fragment includes a Fe moiety that comprises the substitution mutations M428L/N434S. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a 1332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D (e.g., comprises S at 239). In certain embodiments, an antibody or antigen-binding fragment includes a Fc polypeptide or fragment thereof that comprises the substitution mutations: M428L/N434S and G236A/S239D/A330L/I332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and 1332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc of the present disclosure comprises two CH2-CH3 polypeptides that associate to form a dimer.

In some embodiments, an antibody or antigen-binding fragment of the present disclosure comprises a human IgG1 antibody. In certain embodiments, the human IgG1 antibody comprises a kappa light chain. In certain embodiments, the human IgG1 antibody comprises a wild-type Fc. In certain other embodiments, the human IgG1 antibody comprises one or more mutations in the Fc. In some embodiments, the human IgG1 antibody comprises M428L and N434S mutations in the Fc. In certain embodiments, the human IgG1 antibody comprises G236A, A330L, and I332E mutations in the Fc. In certain embodiments, the human IgG1 antibody comprises M428L, N434S, G236A, A330L, and 1332E mutations in the Fc. In some embodiments, the human IgG1 antibody does not comprise any other mutations in the Fc, relative to wild-type IgG1 Fc. In some embodiments, the human IgG1 antibody comprises the VH amino acid sequence of SEQ ID NO.:37 and the VL amino acid sequence of SEQ ID NO.:8.

In some embodiments, a presently disclosed antibody or antigen-binding fragment comprises a CH1-CH3 that comprises or consists of the amino acid sequence set forth in SEQ ID NO.47 or 49. In some embodiments, a presently disclosed antibody or antigen-binding fragment comprises a CL that comprises or consists of the amino acid sequence set forth in SEQ ID NO.:48.

In some embodiments, an antibody, or an antigen-binding fragment thereof, is provided that comprises a heavy chain and a light chain, wherein: (i) the heavy chain comprises or consists of (1) a heavy chain variable domain (VH), wherein the VH comprises or consists of the amino acid sequence of SEQ ID NO.: 37, and (2) a CH1-CH3 that comprises or consists of the amino acid sequence set forth in SEQ ID NO.47 or 49; and (ii) the light chain comprises or consists of (1) a light chain variable domain (VL), wherein the VL comprises or consists of the amino acid sequence of SEQ ID NO.:8, and (2) a CL that comprises or consists of the amino acid sequence of SEQ ID NO.:48.

In some embodiments, an antibody, or an antigen-binding fragment thereof, is provided that comprises two heavy chains and two light chains, wherein: (i) each of the two heavy chains comprises or consists of (1) a heavy chain variable domain (VH), wherein the VH comprises or consists of the amino acid sequence of SEQ ID NO.: 37, and (2) a CH1-CH3 that comprises or consists of the amino acid sequence set forth in SEQ ID NO.:47 or 49; and (ii) each of the two light chains comprises or consists of (1) a light chain variable domain (VL), wherein the VL comprises or consists of the amino acid sequence of SEQ ID NO.:8, and (2) a CL that comprises or consists of the amino acid sequence of SEQ ID NO.:48.

In some embodiments, an antibody, or an antigen-binding fragment thereof, is provided that comprises a heavy chain comprising or consisting of the amino acid sequence of SEQ ID NO.:50 or 51 and a light chain comprising or consisting of SEQ ID NO.:52.

In some embodiments, an antibody, or an antigen-binding fragment thereof, is provided that comprises two heavy chains, each comprising or consisting of the amino acid sequence of SEQ ID NO.:50 or 51, and two light chains, each comprising or consisting of SEQ ID NO.:52.

In some embodiments, an antibody or antigen-binding fragment comprises a heavy chain comprising or consisting of the amino acid sequence of SEQ ID NO.:56. In some embodiments, an antibody or antigen-binding fragment comprises a heavy chain comprising or consisting of the amino acid sequence of SEQ ID NO.:56, and a light chain comprising or consisting of the amino acid sequence of SEQ ID NO.:52. In some embodiments, an antibody or antigen-binding fragment comprises two heavy chains, each comprising or consisting of the amino acid sequence of SEQ ID NO.:56, and two light chains, each comprising or consisting of the amino acid sequence of SEQ ID NO.:52.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. P Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human (i.e., sequences that are encoded by human antibody-encoding genes). However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

In some embodiments, various pharmacokinetic (“PK”) parameters are used to describe or characterize the antibodies or antigen-binding fragments provided herein. Details regarding collection of antibody serum concentrations for purpose of evaluating PK parameters are described in association with the Examples herein. The term “t1/2” or “half-life” refers to the elimination half-life of the antibody or antigen-binding fragment included in the pharmaceutical composition administered to a subject. The term “Ciast” generally refers to the last measurable plasma concentration (i.e., subsequent thereto, the substance is not present at a measurable concentration in plasma).

In certain embodiments, an antibody or antigen-binding fragment of the present disclosure is capable of preventing and/or attenuating an infection by: (i) a H1N1 IAV, wherein, optionally, the H1N1 IAV comprises A/PR8/34; and/or (ii) a H3N2 IAV, wherein, optionally, the H3N2 IAV comprises A/Hong Kong/68.

In certain embodiments, an antibody or antigen-binding fragment of the present disclosure is capable of preventing or reducing weight loss in a subject having an IAV infection, optionally for (i) up to 15 days, or (ii) for 15 or more days, following administration of an effective amount of the antibody or antigen-binding fragment, wherein preventing or reducing weight loss is with reference to an untreated reference subject having the IAV infection. In certain embodiments, an antibody or antigen-binding fragment of the present disclosure is capable of preventing a loss in body weight of greater than 10% in a subject having an IAV infection, wherein a loss in body weight is determined by reference to the subject's body weight just prior to or in an early stage of the IAV infection. In certain embodiments, an antibody or antigen-binding fragment of the present disclosure is capable of extending survival of a subject having an IAV infection, as compared to survival of an untreated reference subject having the IAV infection.

In certain embodiments, an antibody or antigen-binding fragment of the present disclosure has an in vivo half-life in a mouse (e.g., a tg32 mouse): (i) in a range of: from about 7 days to about 12.2 days, from about 8 days to about 11 days, from about 8.5 days to about 10.5 days, or from about 9 days to about 10.5 days; (ii) of between 8 days and 11 days, or between 8.5 days and 10.5 days, or between 9 days and 10 days; (iii) of 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, or 12.2 days; (iv) in a range of from about 9.5 days to about 12.5 days, from about 10 days to 11.5 days; (v) of from 10 days to 11 days, or from 10.5 days to 11 days; (vi) between 10 days and 11.5 days, or between 10.5 days and 11 days, or between 10 days and 11 days; and/or (vii) of 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5 days.

In certain embodiments, an antibody or antigen-binding fragment of the present disclosure specifically binds to the HA and does not bind to, or does not specifically bind to, a non-HA target.

In certain embodiments, an antibody or antigen-binding fragment of the present disclosure capable of binding to any one or more of the following IAV subtypes: H1, H2, H3, H4, H5, H8, H9, H10, H11, H12, H13, H14, H15, H17, and H18.

In certain embodiments, an antibody or antigen-binding fragment of the present disclosure is capable of preventing or attenuating an IAV infection a subject. In certain embodiments, an antibody or antigen-binding fragment of the present disclosure is capable of neutralizing infection by: (i) a H1N1 IAV, wherein, optionally, the H1N1 IAV comprises any one or more of: A/California/07/2009, A/PR/8/34, and A/Solomon Islands/3/06; and (ii) a H3N2 IAV, wherein, optionally, the H3N2 IAV comprises any one or more of: A/Aichi/2/68, A/Brisbane/10/07, and A/Hong Kong/68.

In certain embodiments, an antibody or antigen-binding fragment of the present disclosure is capable of: (iii) neutralizing a H1N1 IAV infection, optionally by A/California/07/2009, with an IC50 in a range of from about 10³ ng/mL to about 10⁴ ng/mL, optionally in a range of from 2,000 ng/mL to 6,000 ng/mL (e.g., 2,000, 2,500, 3,000, 3,500, 4,000, 4,500, 5,000, 5,500, or 6,000 ng/mL); and/or (iv) neutralizing a H3N2 IAV infection, optionally by A/Aichi/2/68, with an IC50 in a range of from 103 ng/mL to 10⁴ ng/mL, optionally in a range of from 3,000 ng/mL to 10,000 ng/mL.

In certain embodiments, an antibody or antigen-binding fragment of the present disclosure is capable of neutralizing infection by: (i) a Group 1 IAV, wherein, optionally, the Group 1 IAV comprises or is a H5 IAV, wherein, further optionally, the H5 IAV comprises or is H5/VN/11/94 pp; and (ii) a Group 2 IAV, wherein, optionally, the Group 2 IAV comprises or is a H7 IAV, wherein, further optionally, the H7 IAV comprises or is H7/IT/99 pp, wherein, optionally, neutralization of infection is as determined using a virus pseudotyped with the IAV.

In certain embodiments, an antibody or antigen-binding fragment of the present disclosure is capable of: (iii) neutralizing an infection by a Group 1 IAV, optionally H5/VN/11/94, with an IC50 in a range of from about 1 ng/mL to about 8 ng/mL (e.g., about 1, 2, 3, 4, 5, 6, 7, or 8 ng/nL); and (iv) neutralizing an infection by a Group 2 IAV, optionally H7/IT/99 pp, with an IC50 in a range of from about 10 ng/mL to about 200 ng/mL.

In certain embodiments, an antibody or antigen-binding fragment of the present disclosure is capable of activating a human FcγRIIIa, which is optionally a F158 allele. In certain embodiments, an antibody or antigen-binding fragment of the present disclosure is capable of activating a human a human FcγRIIa, which is optionally a H131 allele. In some embodiments, activation is as determined using a host cell (optionally a Jurkat cell) comprising: (i) (a) the human FcγRIIIa (optionally a F158 allele), and/or (b) the human FcγRIIa (optionally a H131 allele); and (ii) a NFAT expression control sequence operably linked to a sequence encoding a reporter, such as a luciferase reporter, following incubation (e.g., of 20 hours) of the antibody or antigen-binding fragment with a target cell (e.g., a A549 cell) infected with an IAV.

In certain further embodiments, activation is as determined following incubation of the antibody or antigen-binding fragment with: (1) the target cell infected with a H1N1 IAV, wherein, optionally, the H1N1 IAV is A/PR/8/34, and wherein, optionally, the infection has a multiplicity of infection (MOI) of 6; and/or (2) the target cell infected with a H3N2 IAV, wherein, optionally, the H3N2 IAV is A/Aichi/2/68, and wherein, optionally, the infection has a multiplicity of infection (MOI) of 18.

In certain embodiments, an antibody or antigen-binding fragment of the present disclosure is capable of neutralizing infection by a H5 pseudovirus with a IC50 of less than 4.5 ng/mL, 4.0 ng/mL or less, 3.0 ng/mL or less, 2.5 ng/mL or less, 2.0 ng/mL or less, 1.5 ng/mL or less, 1.0 ng/mL or less, 0.9 ng/mL or less, 0.8 ng/mL or less, 0.7 ng/mL or less, 0.6 ng/mL or less, 0.5 ng/mL or less, 0.4 ng/mL or less, 0.3 ng/mL or less, or 0.2 ng/mL or less.

In certain embodiments, an antibody or antigen-binding fragment of the present disclosure is capable of neutralizing infection by a H5 pseudovirus with an IC50 in a range of: from about 0.2 ng/mL to about 4.5 ng/mL, or from about 0.2 ng/mL to about 4.0 ng/mL, or from about 0.2 ng/mL to about 3.5 ng/mL, or from about 0.2 ng/mL to about 3.0 ng/mL, or from about 0.2 ng/mL to about 2.5 ng/mL, or from about 0.2 ng/mL to about 2.0 ng/mL, or from about 0.2 ng/mL to about 1.5 ng/mL, or from about 0.2 ng/mL to about 1.0 ng/mL, or from about 0.2 ng/mL to about 0.5 ng/mL, or from about 0.5 ng/mL to about 4.5 ng/mL, or from about 0.5 ng/mL to about 4.0 ng/mL, or from about 0.5 ng/mL to about 3.5 ng/mL, or from about 0.5 ng/mL to about 3.0 ng/mL, or from about 0.5 ng/mL to about 2.5 ng/mL, or from about 0.5 ng/mL to about 2.0 ng/mL, or from about 0.5 ng/mL to about 1.5 ng/mL, or from about 0.5 ng/mL to about 1.0 ng/mL, or from about 1.0 ng/mL to about 4.5 ng/mL, or from about 1.0 ng/mL to about 4.0 ng/mL, or from about 1.0 ng/mL to about 3.5 ng/mL, or from about 1.0 ng/mL to about 3.0 ng/mL, or from about 1.0 ng/mL to about 2.5 ng/mL, or from about 1.0 ng/mL to about 2.0 ng/mL, or from about 1.0 ng/mL to about 1.5 ng/mL, or from about 1.5 ng/mL to about 4.5 ng/mL, or from about 1.5 ng/mL to about 4.0 ng/mL, or from about 1.5 ng/mL to about 3.5 ng/mL, or from about 1.5 ng/mL to about 3.0 ng/mL, or from about 1.5 ng/mL to about 2.5 ng/mL, or from about 1.5 ng/mL to about 2.0 ng/mL, or from about 2.0 ng/mL to about 4.5 ng/mL, or from about 2.0 ng/mL to about 4.0 ng/mL, or from about 2.0 ng/mL to about 3.5 ng/mL, or from about 2.0 ng/mL to about 3.0 ng/mL, or from about 2.0 ng/mL to about 2.5 ng/mL, or from about 2.5 ng/mL to about 4.5 ng/mL, or from about 2.5 ng/mL to about 4.0 ng/mL, or from about 2.5 ng/mL to about 3.5 ng/mL, or from about 2.5 ng/mL to about 3.0 ng/mL, or from about 3.0 ng/mL to about 4.5 ng/mL, or from about 3.0 ng/mL to about 4.0 ng/mL, or from about 3.0 ng/mL to about 3.5 ng/mL, or from about 3.5 ng/mL to about 4.5 ng/mL, or from about 3.5 ng/mL to about 4.0 ng/mL, or from about 4.0 ng/mL to about 4.5 ng/mL.

In certain embodiments, an antibody or antigen-binding fragment of the present disclosure is capable of neutralizing infection by a H5 pseudovirus with a IC50 of about 0.6 ng/mL, about 0.5 ng/mL, about 0.4 ng/mL, about 0.3 ng/mL, or about 0.2 ng/mL.

In certain embodiments, an antibody or antigen-binding fragment of the present disclosure is capable of neutralizing infection by a H5 pseudovirus with a IC50 of 0.7 ng/mL or less, 0.6 ng/mL or less, 0.5 ng/mL or less, 0.4 ng/mL or less, 0.3 ng/mL or less, or 0.20 ng/mL or less.

In certain embodiments, an antibody or antigen-binding fragment of the present disclosure is capable of neutralizing infection by: (i) a H1N1 IAV with a IC50 in a range of from about 850 ng/mL to about 4,500 ng/mL, and/or with a IC90 in a range of from about 1,000 ng/mL to about 5,400 ng/mL; and/or (ii) a H3N2 IAV with a IC50 in a range of from about 300 ng/mL to about 2,800 ng/mL, and/or with a IC90 in a range of from about 350 ng/mL to about 7,600 ng/mL.

In certain embodiments, an antibody or antigen-binding fragment of the present disclosure is capable of neutralizing infection by: (i) a H1N1 IAV with a IC50 in a range of from about 880 ng/mL to about 1,120 ng/mL, and/or with a IC90 in a range of from about 1,050 ng/mL to about 1,680 ng/mL; (ii) a H3N2 IAV with a IC50 in a range of from about 300 ng/mL to about 2,100 ng/mL and/or with a IC90 in a range of from about 350 ng/mL to about 2,700 ng/mL; (iii) a H1N1 IAV with a IC50 in a range of from about 1,100 ng/mL to about 2,700 ng/mL, and/or with a IC90 in a range of from about 1,040 ng/mL to about 4,540 ng/mL; (iv) a H3N2 IAV with a IC50 in a range of from about 500 ng/mL to about 2,420 ng/mL and/or with a IC90 in a range of from about 680 ng/mL to about 4,570 ng/mL; (v) a H1N1 IAV with a IC50 in a range of from about 1,030 ng/mL to about 1,680 ng/mL, and/or with a IC90 in a range of from about 1,780 ng/mL to about 4,760 ng/mL; (vi) a H3N2 IAV with a IC50 in a range of from about 440 ng/mL to about 2,540 ng/mL and/or with a IC90 in a range of from about 450 ng/mL to about 4,250 ng/mL; (vii) a H1N1 IAV with a IC50 in a range of from about 1,950 ng/mL to about 2,000 ng/mL, and/or with a IC90 in a range of from about 2,420 ng/mL to about 5,400 ng/mL; and/or (viii) a H3N2 IAV with a IC50 in a range of from about 880 ng/mL to about 2,820 ng/mL and/or with a IC90 in a range of from about 1,170 ng/mL to about 7,630 ng/mL.

In certain embodiments, an antibody or antigen-binding fragment of the present disclosure is capable of neutralizing infection by: (i) a H1N1 A/PR/8/34 IAV with a IC50 in a range of from about 850 ng/mL to about 2000 ng/mL (e.g., about 880 ng/mL, about 1,000 ng/mL, about 1100 ng/mL, about 2,000 ng/mL), and/or with a IC90 in a range of from about 1050 ng/mL to about 2,400 ng/mL (e.g., about 1,050 ng/mL, about 1850 ng/mL, about 1,780 ng/mL, about 2,400 ng/mL); (ii) a H1N1 A/Solomon Islands/3/06 IAV with a IC50 in a range of from about 1,100 ng/mL to about 2,700 ng/mL (e.g., about 1,100 ng/mL, about 1,680 ng/mL, about 1950 ng/mL, about 2,700 ng/mL) and/or with a IC90 in a range of from about 1680 ng/mL to about 5,400 ng/mL (e.g., about 1680 ng/mL, about 4,500 ng/mL, about 4700 ng/mL, about 5,400 ng/mL); (iii) a H3N2 A/Aichi/2/68 IAV with a IC50 in a range of from about 2,100 ng/mL to about 2,900 ng/mL (e.g., about 2,100 ng/mL, about 2,400 ng/mL, about 2,500 ng/mL, about 2,800 ng/mL) and/or with a IC90 in a range of from about 2,700 ng/mL to about 7,600 ng/mL (e.g., about 2,700 ng/mL, about 4,200, about 4,500 ng/mL, about 7,600 ng/mL); (iv) a H3N2 A/Brisbane/10/07 IAV with a IC50 in a range of from about 300 ng/mL to about 880 ng/mL (e.g., about 300 ng/mL, about 440 ng/mL, about 500 ng/mL, about 880 ng/mL) and/or with a IC90 in a range of from about 350 ng/mL to about 1,200 ng/mL (e.g., about 350 ng/mL, about 450 ng/mL, about 680 ng/mL, about 1,200 ng/mL); (v) a H1N1 A/CAL/09 IAV with a IC50 in a range of from about 3,100 ng/mL to about 4,500 ng/mL (e.g., about 3,100 ng/mL, about 3,600 ng/mL, about 4,300 ng/mL, about 4,500 ng/mL) and/or with a IC90 in a range of from about 350 ng/mL to about 1,200 ng/mL (e.g., about 350 ng/mL, about 450 ng/mL, about 680 ng/mL, about 1,200 ng/mL); and/or (vi) a H3N2 A/HK/68 IAV with a IC50 in a range of from about 2,000 ng/mL to about 3,000 ng/mL (e.g., about 2,000 ng/mL, about 2,100 ng/mL, about 2,200 ng/mL, about 2,300 ng/mL, about 2,400 ng/mL, about 2,500 ng/mL, about 2,600 ng/mL, about 2,700 ng/mL, about 2,800 ng/mL, about 2,900 ng/mL, about 3,000 ng/mL), preferably in a range of from about 2,100 ng/mL to about 2,500 ng/mL.

In certain embodiments, an antibody or antigen-binding fragment of the present disclosure is capable of neutralizing infection by: (i) a H1N1 A/PR/8/34 IAV with a IC50 in a range of: from about 860 to about 920 ng/mL, from about 1,000 to about 1,060 ng/mL, from about 1,080 ng/mL to about 1,140 ng/mL, or from about 1,970 ng/mL to about 2,030 ng/mL, and/or with a IC90 in a range of: from about 1,015 ng/ml to about 1,075 ng/mL, from about 1,750 ng/mL to about 1,810 ng/mL, from about 1,750 ng/mL to about 1,830 ng/mL, or from about 2,390 ng/mL to about 2,450 ng/mL; (ii) a H1N1 A/Solomon Islands/3/06 IAV with a IC50 in a range of from about 1,100 ng/mL to about 2,700 ng/mL (e.g., about 1,100 ng/mL, about 1,680 ng/mL, about 1950 ng/mL, about 2,700 ng/mL) and/or with a IC90 in a range of from about 1680 ng/mL to about 5,400 ng/mL (e.g., about 1680 ng/mL, about 4,500 ng/mL, about 4700 ng/mL, about 5,400 ng/mL); (iii) a H3N2 A/Aichi/2/68 IAV with a IC50 in a range of from about 2,100 ng/mL to about 2,900 ng/mL (e.g., about 2,100 ng/mL, about 2,400 ng/mL, about 2,800 ng/mL) and/or with a IC90 in a range of from about 2,700 ng/mL to about 7,600 ng/mL (e.g., about 2,700 ng/mL, about 4,200, about 4,500 ng/mL, about 7,600 ng/mL); and/or (iv) a H3N3 A/Brisbane/10/07 IAV with a IC50 in a range of from about 300 ng/mL to about 880 ng/mL (e.g., about 300 ng/mL, about 440 ng/mL, about 500 ng/mL, about 88 ng/mL) and/or with a IC90 in a range of from about 350 ng/mL to about 1,200 ng/mL (e.g., about 350 ng/mL, about 450 ng/mL, about 680 ng/mL, about 1,200 ng/mL).

In certain embodiments, an antibody or antigen-binding fragment of the present disclosure is capable of binding to any one or more of the following H3N2 IAV subtypes: A/Babol/36/2005; A/Hong Kong/CUHK31987/2011; A/Texas/50/2012; A/Wisconsin/67/2005; A/Netherlands/178/1995; A/Johannesburg/33/1994; A/Guangdong-Luohu/1256/2009; A/California/7/2004; A/Hanoi/EL134/2008; A/Wuhan/359/1995; A/Victoria/210/2009; A/Philippines/472/2002; A/Hanoi/EL201/2009; AVictoria/210/2009; A/Missouri/09/2014; A/Perth/16/2009; A/Wyoming/03/2003; A/Moscow/10/1999; A/Sydney/5/1997; A/Nanchang/933/1995; A/Beijing/32/92; A/Aichi/2/1968; A/Brisbane/10/2007; and A/Switzerland/9715293/2013.

In certain embodiments, an antibody or antigen-binding fragment of the present disclosure is capable of binding to the one or more H3N2 IAV subtype(s) with a logEC50 (ng/mL) in a range of: from about 0.1 to about 6, from about 0.1 to about 5.5, from about 1 to about 5, from about 0.1 to about 4.5, from about 0.1 to about 4.0, from about 0.1 to about 3.5, from about 0.1 to about 3, from about 0.1 to about 2.5, from about 0.1 to about 2.0, from 0.1 to about 1.5, from 0.1 to about 1.0, or of about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 ng/mL, wherein the binding is as determined by ELISA.

In certain embodiments, an antibody or antigen-binding fragment of the present disclosure is capable of binding to one or more of (i)-(iv): (i) a H1 HA, which optionally comprises any one or more of: A/England/195/2009; A/Brisbane/59/2007; A/Solomon Islands/3/2006; A/New Caledonia/20/99; A/Texas/36/1991; A/Taiwan/01/1986; A/New Jersey/8/1976; A/Albany/12/1951; A/Fort Monmouth/1/1947; A/New York/1/1918; A/Puerto Rico/8/34; and A/California/07/2009; (ii) a H2 HA, optionally comprising A/Japan/305/1957; (iii) a H5 HA, optionally comprising A/Vietnam/1194/2004; and (iv) a H9 HA, optionally comprising A/Hong Kong/1073/99.

In certain embodiments, an antibody or antigen-binding fragment of the present disclosure binds to H5 HA and/or to H7 HA with a KD of less than 1.0E-12 M, less than 1.0E-11 M, less than 1.0E-10 M, less than 1.0E-9 M, less than 1.0E-8 M, or less than 1.0E-7 M, or of 1.0E-8M or less, of 1.0E-9M or less, of 1.0E-10 or less, of 1.0E-11 or less, or 1.0E-12 or less (e.g., as determined by Bio-Layer Interferometry (BLI)).

In certain embodiments, an antibody or antigen-binding fragment of the present disclosure is capable of binding to one or more of (i)-(iv) with a logEC50 (ng/mL) in a range: from about 0.05 to about 1.5, from about 0.05 to about 1.4, from about 0.05 to about 1.3, from about 0.05 to about 1.2, from about 0.05 to about 1.1, from about 0.05 to about 1.0, from about 0.05 to about 0.9, from about 0.05 to about 0.8, from about 0.05 to about 0.7, from about 0.05 to about 0.6, from about 0.05 to about 0.5, from about 0.1 to about 1, or about 1.3, about 1.2, about 1.1, about 1.0, about 0.9, about 0.8, about 0.7, about 0.6, about 0.5, about 0.4, about 0.3, about 0.2, about 0.1, or about 0.05, wherein the binding is as determined by ELISA.

### Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain).

In certain embodiments, the polynucleotide comprises deoxyribonucleic acid (DNA) or ribonucleic acid (RNA), wherein the RNA optionally comprises messenger RNA (mRNA).

In some embodiments, the polynucleotide comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises N1-methylpseudouridine.

In certain embodiments, the polynucleotide is codon-optimized for expression in a host cell (e.g., a human cell or a CHO cell). Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In certain embodiments, a polynucleotide is provided that comprises a polynucleotide having at least 50%, (e.g., 50%, 55%, 60%, 65%, 70%, 75%, 80%, 85%, 90%, 91%, 92%, 93%, 94%, 95%, 96%, 97%, 98%, 99%, or more) identity to the polynucleotide sequence according to any one or more of SEQ ID NOs.: 1, 6, 7, 12, 25, 27, 30, 33, 36, 13, 18, 19, 24, 38, and 40.

In certain embodiments, a polynucleotide is provided that comprises a (i) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:6 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (ii) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:25 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (iii) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:27 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (iv) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:30 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (v) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:33 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (vi) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:36 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (vii) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:18 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:24; (viii) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:38 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:24; or (ix) a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:40 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:24.

In some embodiments, a polynucleotide encoding an antibody heavy chain comprises or consists of the polynucleotide sequence of SEQ ID NO.:54.

In some embodiments, a polynucleotide encoding an antibody light chain comprises or consists of the polynucleotide sequence of SEQ ID NO.:55. In some embodiments, a polynucleotide encoding an antibody heavy chain comprises or consists of the polynucleotide sequence of SEQ ID NO.:54, and a polynucleotide encoding an antibody light chain comprises or consists of the polynucleotide sequence of SEQ ID NO.:55.

In any of the presently disclosed embodiments, the polynucleotide can comprise deoxyribonucleic acid (DNA) or ribonucleic acid (RNA). In some embodiments, the RNA comprises messenger RNA (mRNA).

Vectors are also provided, wherein the vectors comprise or contain a polynucleotide as disclosed herein (e.g., a polynucleotide that encodes an antibody or antigen-binding fragment that binds to IAV HA). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J InfectDis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH1, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVaxl, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a polynucleotide (e.g., mRNA) is provided that encodes a heavy chain and a light chain of an antibody or antigen-binding fragment thereof. In some embodiments, a polynucleotide (e.g., mRNA) is provided that encodes two heavy chains and two light chains of an antibody or antigen-binding fragment thereof. See, e.g. Li, JQ., Zhang, ZR., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters.

In some embodiments, a polynucleotide or replicon can translate simultaneously the heavy chain (or VH, or VH+1) and the light chain (or VL, or VL+CL) of an antibody or antigen-binding fragment thereof. In some embodiments, a method is provided that comprises delivering to a subject such a polynucleotide or replicon.In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells, such as human B cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, YO cells, Sp2/0 cells. NSO cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glysocylation or fucosylation), or a lack thereof, on the antibody or antigen-binding fragment that is not present in a native state of the antibody or antigen-binding fragment (or in a native state of a parent antibody from which the antibody or antigen binding fragment was engineered or derived). Such a PTM, or a lack thereof, may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a host cell can comprise one or more post-translational modification, or can include fewer post-translational modification(s), such that it is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodopterafrugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodopterafrugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a YO cell, a Sp2/0 cell, a NSO cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise a presently disclosed antibody, antigen-binding fragment, polynucleotide, vector, or host cell, singly or in any combination, and can further comprise a pharmaceutically acceptable carrier, excipient, or diluent. Such compositions, as well as carriers, excipients, and diluents, are discussed in further detail herein.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. NanomedNanobiotechnol. 11(2): e1530 (2019)). Principles, reagents, and techniques for designing appropriate mRNA and and formulating mRNA-LNP and delivering the same are described in, for example, Pardi et al. (J ControlRelease 217345-351 (2015)); Thess et al. (Mol Ther 23: 1456-1464 (2015)); Thran et al. (EMBO MolMed 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

In certain embodiments, a composition comprises a first antibody or antigen-binding fragment of the present disclosure and a second antibody or antigen-binding fragment of the present disclosure, wherein of the first antibody or antigen-binding fragment and the second antibody or antigen-binding fragment are different.

### Methods and Uses

Also provided herein are methods for use of an antibody or antigen-binding fragment, nucleic acid, vector, cell, or composition of the present disclosure in the diagnosis of IAV infection (e.g., in a human subject, or in a sample obtained from a human subject). Methods of diagnosis (e.g., in vitro, ex vivo) may include contacting an antibody, antibody fragment (e.g., antigen binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by IAV. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a IAV infection (i.e., in a statistically significant manner). In certain embodiments, therapeutic or prophylactic/preventive benefit includes a reduced duration of hospitalization for treatment of a IAV infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

Accordingly, in certain embodiments, methods are provided for treating a IAV infection in a subject, wherein the methods comprise administering to the subject an effective amount of an antibody, antigen-binding fragment, polynucleotide, vector, host cell, or composition as disclosed herein.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with a IAV infection. These include, but are not limited to, age, occupation, general health, pre-existing health conditions, locale, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age can be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a subject treated according to the present disclosure has received a vaccine for IAV and the vaccine is determined to be ineffective, e.g., by post-vaccine infection or symptoms in the subject, by clinical diagnosis or scientific or regulatory consensus.

Prophylaxis of infection with influenza A virus refers in particular to prophylactic settings, wherein the subject was not diagnosed with infection with influenza A virus (either no diagnosis was performed or diagnosis results were negative) and/or the subject does not show or experience symptoms of infection with influenza A virus. Prophylaxis of infection with influenza A virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza A virus is also particularly useful in subjects at greater risk acquiring influenza A virus infection, e.g., due to increased exposure, for example subjects working or staying in public areas, in particular health care workers.

In certain embodiments, treatment is administered as peri-exposure or pre-exposure prophylaxis.

In therapeutic settings, in contrast, the subject is typically infected with influenza A virus, diagnosed with influenza A virus infection, and/or showing symptoms of influenza A virus infection. Of note, the terms “treatment” and “therapy”/“therapeutic” of influenza A virus infection can refer to (complete) cure as well as attenuation/reduction of influenza A virus infection and/or related symptoms (e.g., attenuation/reduction of severity of infection and/or symptoms, number of symptoms, duration of infection and/or symptoms, or any combination thereof).

It will be understood that reference herein to a reduced number and/or severity of symptoms, which reduction results from administration of a presently disclosed pharmaceutical composition, describes a comparison with a reference subject who did not receive a disclosed pharmaceutical composition. A reference subject can be, for example, (i) the same subject during an earlier period of time (e.g., a prior influenza A virus season), (ii) a subject of a same or a similar: age or age group; gender; pregnancy status; chronic medical condition (such as chronic cardiac, pulmonary, renal, metabolic, neurodevelopmental, liver or hematologic diseases) or lack thereof; and/or immunosuppressive condition or lack thereof, or (iii) a typical subject within a population (e.g., local, regional, or national, including of a same or similar age or age range and/or general state of health) during an influenza A virus season. Prophylaxis can be determined by, for example, the failure to develop a diagnosed influenza A infection and/or the lack of symptoms associated with influenza A infection during a part of a full influenza A season, or over a full influenza A season.

In certain embodiments, the methods provided herein include administering a therapeutically effective amount of a composition according to the present disclosure to a subject at immediate risk of influenza A infection. An immediate risk of influenza A infection typically occurs during an influenza A epidemic. Influenza A viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza A epidemic is high during November, December, January, February and March, while in the southern hemisphere the risk of an influenza A epidemic is high during May, June, July, August and September.

In some embodiments, treatment and/or prevention comprises post-exposure prophylaxis.

In some embodiments, the subject has received, is receiving, or will receive an antiviral agent. In some embodiments, the antiviral agent comprises a neuraminidase inhibitor, an influenza polymerase inhibitor, or both. In certain embodiments, the antiviral agent comprises oseltamivir, lanamivir, peramivir, zanamivir, baloxavir, or any combination thereof.

Typical routes of administering the presently disclosed compositions include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder.

Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat an influenza infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by IAV.

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In some embodiments, an antibody (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In a related aspect, uses of the presently disclosed antibodies, antigen-binding fragments, vectors, host cells, and compositions are provided.

In certain embodiments, an antibody, antigen-binding fragment, polynucleotide, vector, host cell, or composition is provided for use in a method of treating a IAV infection in a subject.

In certain embodiments, an antibody, antigen-binding fragment, polynucleotide, vector, host cell, or composition is provided for use in a method of manufacturing or preparing a medicament for treating a IAV infection in a subject.

The present disclosure also provides the following non-limiting embodiments.

Embodiment 1. An antibody, or antigen-binding fragment thereof, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein:
(i) the CDRH1 comprises or consists of the amino acid sequence of any one of SEQ ID NOs.: 32, 3, or 15, or a functional variant thereof comprising one, two, or three acid substitutions, one or more of which substitutions is optionally a conservative substitution and/or is a substitution to a germline-encoded amino acid; and/or (ii) the CDRH2 comprises or consists of the amino acid sequence of any one of SEQ ID NOs.: 35, 4, 29, 16, and 42, or a functional variant thereof comprising one, two, or three amino acid substitutions, one or more of which substitutions is optionally a conservative substitution and/or is a substitution to a germline-encoded amino acid; and/or (iii) the CDRH3 comprises or consists of the amino acid sequence of SEQ ID NO.: 5 or 17, or a functional variant thereof comprising one, two, or three amino acid substitutions, one or more of which substitutions is optionally a conservative substitution and/or is a substitution to a germline-encoded amino acid; and/or (iv) the CDRL1 comprises or consists of the amino acid sequence of SEQ ID NO.: 9 or 21, or a functional variant thereof comprising one, two, or three amino acid substitutions, one or more of which substitutions is optionally a conservative substitution and/or is a substitution to a germline-encoded amino acid; and/or (v) the CDRL2 optionally comprises or consists of the amino acid sequence of SEQ ID NO.: 10 or 22, or a functional variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence of SEQ ID NO.: 11 or 23, or a functional variant thereof comprising having one, two, or three amino acid substitutions, one or more of which substitutions is optionally a conservative substitution and/or is a substitution to a germline-encoded amino acid,
wherein the antibody or antigen-binding fragment is capable of binding to an influenza A virus (IAV) hemagglutinin (HA).

Embodiment 2. The antibody or antigen-binding fragment of Embodiment 1, wherein the antibody or antigen-binding fragment is capable of binding to an influenza A virus (IAV) hemagglutinin (HA on a cell surface of a host cell and/or on a virion.

Embodiment 3. The antibody or antigen-binding fragment of Embodiment 1 or 2, which is capable of neutralizing an IAV infection in an in vitro model of infection and/or in an in vivo animal model of infection and/or in a human, wherein, optionally, the in vitro model of infection comprises a target cell and a pseudovirus or a target cell and a live virus.

Embodiment 4. The antibody or antigen-binding fragment of any one of Embodiments 1-3, comprising CDRH1, CDRH2, CDRH3, CDRL1, CDRL2, and CDRL3 amino acid sequences of SEQ ID NOs.: (i) 32, 35, 5, and 9-11, respectively; (ii) 3, 29, 5 and 9-11, respectively; (iii) 32, 4, 5 and 9-11, respectively; (iv) 3, 35, and 9-11, respectively; (v) 3-5 and 9-11, respectively; (vi) 15-17 and 21-23, respectively; or (vii) 15, 42, 17 and 21-23, respectively.

Embodiment 5. The antibody or antigen-binding fragment of any one of Embodiments 1-3, comprising CDRH1, CDRH2, CDRH3, CDRL1, CDRL2, and CDRL3 amino acid sequences of SEQ ID NOs.: (i) 3, 29, 5 and 9-11, respectively; (ii) 3, 35, 17 and 9-11, respectively; or (iii) 32, 35, 17, and 9-11, respectively.

Embodiment 6. The antibody or antigen-binding fragment of any one of Embodiments 1-5, wherein:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 37, 2, 26, 28, 31, 34, 14, 39 and 41, wherein sequence variation with reference to SEQ ID NO.: 37, 2, 26, 28, 31, 34, 14, 39 or 41, respectively, is optionally comprised in one or more framework region and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 8 or 20, wherein sequence variation with respect to SEQ ID NO.:8 or 20, respectively, is optionally comprised in one or more framework regions and/or sequence variation comprises one or more substitution to a germline-encoded amino acid.

Embodiment 7. The antibody or antigen-binding fragment of any one of Embodiments 1-6, wherein:
(i) the VH comprises or consists of an amino acid sequence having at least 80% identity to the amino acid sequence of any one of SEQ ID NOs.: 37, 2, 26, 28, 31, 34, 14, 39 and 41, and the VL comprises or consists of an amino acid sequence having at least 80% identity to the amino acid sequence of SEQ ID NO.:8; or
(ii) the VH comprises or consists of an amino acid sequence having at least 80% identity to the amino acid sequence of any one of SEQ ID NOs.: 37, 2, 26, 28, 31, 34, 14, 39 and 41, and the VL comprises or consists of an amino acid sequence having at least 80% identity to the amino acid sequence of SEQ ID NO.:20.

Embodiment 8. The antibody or antigen-binding fragment of any one of Embodiments 1-7, wherein the VH and the VL comprise or consist of the amino acid sequences according to SEQ ID NOs.: (i) 37 and 8, respectively; (ii) 26 and 8, respectively; (iii) 28 and 8, respectively; (iv) 31 and 8, respectively; (v) 34 and 8, respectively; (vi) 2 and 8, respectively; (vii) 14 and 20, respectively; (viii) 39 and 20, respectively; or (ix) 41 and 20, respectively.

Embodiment 9. The antibody or antigen-binding fragment of any one of Embodiments 1-7, wherein the VH and the VL comprise or consist of the amino acid sequences according to SEQ ID NOs.: (i) 2 and 20, respectively; (ii) 26 and 20, respectively; (iii) 28 and 20, respectively; (iv) 31 and 20, respectively; (v) 34 and 20, respectively; (vi) 37 and 20, respectively; (v) 14 and 8, respectively; (vi) 39 and 8, respectively; or (vii) 41 and 8, respectively.

Embodiment 10. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 32, 35, and 5, respectively, and the CDRL1, CDRL2, and CDRL3 comprise or consist of the amino acid sequences of SEQ ID NOs.: 9-11, respectively, wherein the antibody or antigen-binding fragment is capable of binding to an influenza A virus (IAV) hemagglutinin (HA).

Embodiment 11. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 3, 29, and 5, respectively, and the CDRL1, CDRL2, and CDRL3 comprise or consist of the amino acid sequences of SEQ ID NOs.: 9-11, respectively, wherein the antibody or antigen-binding fragment is capable of binding to an influenza A virus (IAV) hemagglutinin (HA).

Embodiment 12. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 32, 4, and 5, respectively, and the CDRL1, CDRL2, and CDRL3 comprise or consist of the amino acid sequences of SEQ ID NOs.: 9-11, respectively, wherein the antibody or antigen-binding fragment is capable of binding to an influenza A virus (IAV) hemagglutinin (HA).

Embodiment 13. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 3, 35, and 5, respectively, and the CDRL1, CDRL2, and CDRL3 comprise or consist of the amino acid sequences of SEQ ID NOs.: 9-11, respectively, wherein the antibody or antigen-binding fragment is capable of binding to an influenza A virus (IAV) hemagglutinin (HA).

Embodiment 14. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 3-5, respectively, and the CDRL1, CDRL2, and CDRL3 comprise or consist of the amino acid sequences of SEQ ID NOs.: 9-11, respectively, wherein the antibody or antigen-binding fragment is capable of binding to an influenza A virus (IAV) hemagglutinin (HA).

Embodiment 15. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences of SEQ ID NOs.: 15-17, respectively, and the CDRL1, CDRL2, and CDRL3 comprise or consist of the amino acid sequences of SEQ ID NOs.: 21-23, respectively, wherein the antibody or antigen-binding fragment is capable of binding to an influenza A virus (IAV) hemagglutinin (HA).

Embodiment 16. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the CDRH1, CDRH2, and CDRH3 comprise or consist of the amino acid sequences set forth in SEQ ID NOs.: 15, 42, and 17, respectively, and the CDRL1, CDRL2, and CDRL3 comprise or consist of the amino acid sequences of SEQ ID NOs.: 21-23, respectively, wherein the antibody or antigen-binding fragment is capable of binding to an influenza A virus (IAV) hemagglutinin (HA).

Embodiment 17. An antibody, or antigen-binding fragment thereof, comprising: (1) a heavy chain variable domain (VH) comprising the amino acid sequence of SEQ ID NO.:53, the amino acid sequence of any one of SEQ ID NOs.:4, 29, and 35, and the amino acid sequence of any one of SEQ ID NOs.:5 and 17; and (2) a light chain variable domain (VL) comprising the amino acid sequences of SEQ ID NOs.:9-11, wherein the antibody or antigen-binding fragment is capable of binding to an influenza A virus (IAV) hemagglutinin (HA).

Embodiment 18. An antibody, or antigen-binding fragment thereof, comprising a heavy chain variable domain (VH) and a light chain variable domain (VL), wherein: (i) the VH comprises a CDRH1, a CDRH2, and a CDRH3 according to the VH amino acid sequence set forth in any one of SEQ ID NOs.: 37, 2, 26, 28, 31, 34, 14, 39 and 41; and (ii) the VL comprises a CDRL1, a CDRL2, and a CDRL3 according to the VL amino acid sequence set forth in SEQ ID NO.:2, wherein the antibody or antigen-binding fragment is capable of binding to an influenza A virus (IAV) hemagglutinin (HA).

Embodiment 19. An antibody, or antigen-binding fragment thereof, comprising a heavy chain variable domain (VH) and a light chain variable domain (VL), wherein: (i) the VH comprises a CDRH1, a CDRH2, and a CDRH3 according to the VH amino acid sequence set forth in any one of SEQ ID NOs.: 37, 2, 26, 28, 31, 34, 14, 39 and 41; and (ii) the VL comprises a CDRL1, a CDRL2, and a CDRL3 according to the VL amino acid sequence set forth in SEQ ID NO.:8, wherein the antibody or antigen-binding fragment is capable of binding to an influenza A virus (IAV) hemagglutinin (HA).

Embodiment 20. The antibody or antigen-binding fragment of Embodiment 18 or 19, wherein the CDRs are according to the IMGT numbering system.

Embodiment 21. The antibody or antigen-binding fragment of Embodiment 18 or 19, wherein the CDRs are according to the Kabat numbering system.

Embodiment 22. The antibody or antigen-binding fragment of Embodiment 18 or 19, wherein the CDRs are according to the Chothia numbering system.

Embodiment 23. The antibody or antigen-binding fragment of Embodiment 18 or 19, wherein the CDRs are according to the AHo numbering system.

Embodiment 24. The antibody or antigen-binding fragment of Embodiment 18 or 19, wherein the CDRs are according to the North numbering system.

Embodiment 25. The antibody or antigen-binding fragment of Embodiment 18 or 19, wherein the CDRs are according to the Martin numbering system.

Embodiment 26. The antibody or antigen-binding fragment of any one of Embodiments 1-25, wherein the VH is encoded by or derived from VH6-1, DH3-3, and JH6, and/or the VL is encoded by or derived from VK3-20 and JK3.

Embodiment 27. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 37 and the VL comprises or consists of the amino acid sequence of SEQ ID NO.: 8.

Embodiment 28. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 26 and the VL comprises or consists of the amino acid sequence of SEQ ID NO.: 8.

Embodiment 29. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 28 and the VL comprises or consists of the amino acid sequence of SEQ ID NO.: 8.

Embodiment 30. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 31 and the VL comprises or consists of the amino acid sequence of SEQ ID NO.: 8.

Embodiment 31. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 34 and the VL comprises or consists of the amino acid sequence of SEQ ID NO.: 8.

Embodiment 32. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 2 and the VL comprises or consists of the amino acid sequence of SEQ ID NO.: 8.

Embodiment 33. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 14 and the VL comprises or consists of the amino acid sequence of SEQ ID NO.: 20.

Embodiment 34. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 39 and the VL comprises or consists of the amino acid sequence of SEQ ID NO.: 20.

Embodiment 35. An anti-influenza hemagglutinin (HA) antibody, or an antigen-binding fragment thereof, comprising a heavy chain variable domain (VH) and a light chain variable domain (VL), wherein the VH comprises or consists of the amino acid sequence of SEQ ID NO.: 41 and the VL comprises or consists of the amino acid sequence of SEQ ID NO.: 20.

Embodiment 36. The antibody or antigen-binding fragment of any one of Embodiments 1-35, wherein the antibody or antigen-binding fragment is capable of preventing and/or attenuating an infection by: (i) a H1N1 IAV, wherein, optionally, the H1N1 IAV comprises A/PR8/34; and/or (ii) a H3N2 IAV, wherein, optionally, the H3N2 IAV comprises A/Hong Kong/68.

Embodiment 37. The antibody or antigen-binding fragment of any one of Embodiments 1-36, wherein the antibody or antigen-binding fragment is capable of preventing or reducing weight loss in a subject having an IAV infection, optionally for (i) up to 15 days, or (ii) for 15 or more days, following administration of an effective amount of the antibody or antigen-binding fragment, wherein preventing or reducing weight loss is with reference to an untreated reference subject having the IAV infection.

Embodiment 38. The antibody or antigen-binding fragment of any one of Embodiments 1-37, wherein the antibody or antigen-binding fragment is capable of preventing a loss in body weight of greater than 10% in a subject having an IAV infection, wherein a loss in body weight is determined by reference to the subject's body weight just prior to or in an early stage of the IAV infection.

Embodiment 39. The antibody or antigen-binding fragment of any one of Embodiments 1-38, wherein the antibody or antigen-binding fragment is capable of extending survival of a subject having an IAV infection, as compared to survival of an untreated reference subject having the IAV infection.

Embodiment 40. The antibody or antigen-binding fragment of any one of Embodiments 1-39, wherein the antibody or antigen-binding fragment has an in vivo half-life in a mouse (e.g., a tg32 mouse):
(i) in a range of: from about 7 days to about 12.2 days, from about 8 days to about 11 days, from about 8.5 days to about 10.5 days, or from about 9 days to about 10.5 days; (ii) of between 8 days and 11 days, or between 8.5 days and 10.5 days, or between 9 days and 10 days; (iii) of 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, or 12.2 days; (iv) in a range of from about 9.5 days to about 12.5 days, from about 10 days to 11.5 days; (v) of from 10 days to 11 days, or from 10.5 days to 11 days; (vi) between 10 days and 11.5 days, or between 10.5 days and 11 days, or between 10 days and 11 days; and/or (vii) of 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5 days.

Embodiment 41. The antibody or antigen-binding fragment of any one of Embodiments 1-40, which specifically binds to the HA and does not bind to, or does not specifically bind to, a non-HA target.

Embodiment 42. The antibody or antigen-binding fragment of any one of Embodiments 1-41, which is capable of binding to any one or more of the following IAV subtypes: H1, H2, H3, H4, H5, H8, H9, H10, H11, H12, H13, H14, H15, H17, and H18.

Embodiment 43. The antibody or antigen-binding fragment of any one of Embodiments 1-42, wherein the antibody or antigen-binding fragment is capable of preventing or attenuating an IAV infection a subject.

Embodiment 44. The antibody or antigen-binding fragment of any one of Embodiments 1-43, which is capable of neutralizing infection by: (i) a H1N1 IAV, wherein, optionally, the H1N1 IAV comprises any one or more of: A/California/07/2009, A/PR/8/34, and A/Solomon Islands/3/06; and (ii) a H3N2 IAV, wherein, optionally, the H3N2 IAV comprises any one or more of: A/Aichi/2/68, A/Brisbane/10/07, and A/Hong Kong/68.

Embodiment 45. The antibody or antigen-binding fragment of any one of Embodiments 1-44, which is capable of:
(iii) neutralizing a H1N1 IAV infection, optionally by A/California/07/2009, with an IC50 in a range of from about 103 ng/mL to about 104 ng/mL, optionally in a range of from 2,000 ng/mL to 6,000 ng/mL (e.g., 2,000, 2,500, 3,000, 3,500, 4,000, 4,500, 5,000, 5,500, or 6,000 ng/mL); and/or
(iv) neutralizing a H3N2 IAV infection, optionally by A/Aichi/2/68, with an IC50 in a range of from 10³ ng/mL to 10⁴ ng/mL, optionally in a range of from 3,000 ng/mL to 10,000 ng/mL.

Embodiment 46. The antibody or antigen-binding fragment of any one of Embodiments 1-45, which is capable of neutralizing infection by: (i) a Group 1 IAV, wherein, optionally, the Group 1 IAV comprises or is a H5 IAV, wherein, further optionally, the H5 IAV comprises or is H5/VN/11/94 pp; and (ii) a Group 2 IAV, wherein, optionally, the Group 2 IAV comprises or is a H7 IAV, wherein, further optionally, the H7 IAV comprises or is H7/IT/99 pp, wherein, optionally, neutralization of infection is as determined using a virus pseudotyped with the IAV.

Embodiment 47. The antibody or antigen-binding fragment of Embodiment 46, which is capable of: (iii) neutralizing an infection by a Group 1 IAV, optionally H5/VN/11/94, with an IC50 in a range of from about 1 ng/mL to about 8 ng/mL (e.g., about 1, 2, 3, 4, 5, 6, 7, or 8 ng/nL); and (iv) neutralizing an infection by a Group 2 IAV, optionally H7/IT/99 pp, with an IC50 in a range of from about 10 ng/mL to about 200 ng/mL.

Embodiment 48. The antibody or antigen-binding fragment of any one of Embodiments 1-47, which is capable of activating a human FcγRIIIa, which is optionally a F158 allele.

Embodiment 49. The antibody or antigen-binding fragment of any one of Embodiments 1-48, which is capable of activating a human a human FcγRIIa, which is optionally a H131 allele.

Embodiment 50. The antibody or antigen-binding fragment of Embodiment 48 or 49, wherein activation is as determined using a host cell (optionally a Jurkat cell) comprising: (i) (a) the human FcγRIIIa (optionally a F158 allele), and/or (b) the human FcγRIIa (optionally a H131 allele); and (ii) a NFAT expression control sequence operably linked to a sequence encoding a reporter, such as a luciferase reporter, following incubation (e.g., of 20 hours) of the antibody or antigen-binding fragment with a target cell (e.g., a A549 cell) infected with an IAV.

Embodiment 51. The antibody or antigen-binding fragment of Embodiment 50, wherein activation is as determined following incubation of the antibody or antigen-binding fragment with: (1) the target cell infected with a H1N1 IAV, wherein, optionally, the H1N1 IAV is A/PR/8/34, and wherein, optionally, the infection has a multiplicity of infection (MOI) of 6; and/or (2) the target cell infected with a H3N2 IAV, wherein, optionally, the H3N2 IAV is A/Aichi/2/68, and wherein, optionally, the infection has a multiplicity of infection (MOI) of 18.

Embodiment 52. The antibody or antigen-binding fragment of any one of Embodiments 1-51, which is capable of neutralizing infection by a H5 pseudovirus with a IC50 of less than 4.5 ng/mL, 4.0 ng/mL or less, 3.0 ng/mL or less, 2.5 ng/mL or less, 2.0 ng/mL or less, 1.5 ng/mL or less, 1.0 ng/mL or less, 0.9 ng/mL or less, 0.8 ng/mL or less, 0.7 ng/mL or less, 0.6 ng/mL or less, 0.5 ng/mL or less, 0.4 ng/mL or less, 0.3 ng/mL or less, or 0.2 ng/mL or less.

Embodiment 53. The antibody or antigen-binding fragment of any one of Embodiments 1-52, which is capable of neutralizing infection by a H5 pseudovirus with an IC50 in a range of: from about 0.2 ng/mL to about 4.5 ng/mL, or from about 0.2 ng/mL to about 4.0 ng/mL, or from about 0.2 ng/mL to about 3.5 ng/mL, or from about 0.2 ng/mL to about 3.0 ng/mL, or from about 0.2 ng/mL to about 2.5 ng/mL, or from about 0.2 ng/mL to about 2.0 ng/mL, or from about 0.2 ng/mL to about 1.5 ng/mL, or from about 0.2 ng/mL to about 1.0 ng/mL, or from about 0.2 ng/mL to about 0.5 ng/mL, or from about 0.5 ng/mL to about 4.5 ng/mL, or from about 0.5 ng/mL to about 4.0 ng/mL, or from about 0.5 ng/mL to about 3.5 ng/mL, or from about 0.5 ng/mL to about 3.0 ng/mL, or from about 0.5 ng/mL to about 2.5 ng/mL, or from about 0.5 ng/mL to about 2.0 ng/mL, or from about 0.5 ng/mL to about 1.5 ng/mL, or from about 0.5 ng/mL to about 1.0 ng/mL, or from about about 1.0 ng/mL to about 4.5 ng/mL, or from about 1.0 ng/mL to about 4.0 ng/mL, or from about 1.0 ng/mL to about 3.5 ng/mL, or from about 1.0 ng/mL to about 3.0 ng/mL, or from about 1.0 ng/mL to about 2.5 ng/mL, or from about 1.0 ng/mL to about 2.0 ng/mL, or from about 1.0 ng/mL to about 1.5 ng/mL, or from about 1.5 ng/mL to about 4.5 ng/mL, or from about 1.5 ng/mL to about 4.0 ng/mL, or from about 1.5 ng/mL to about 3.5 ng/mL, or from about 1.5 ng/mL to about 3.0 ng/mL, or from about 1.5 ng/mL to about 2.5 ng/mL, or from about 1.5 ng/mL to about 2.0 ng/mL, or from about 2.0 ng/mL to about 4.5 ng/mL, or from about 2.0 ng/mL to about 4.0 ng/mL, or from about 2.0 ng/mL to about 3.5 ng/mL, or from about 2.0 ng/mL to about 3.0 ng/mL, or from about 2.0 ng/mL to about 2.5 ng/mL, or from about 2.5 ng/mL to about 4.5 ng/mL, or from about 2.5 ng/mL to about 4.0 ng/mL, or from about 2.5 ng/mL to about 3.5 ng/mL, or from about 2.5 ng/mL to about 3.0 ng/mL, or from about 3.0 ng/mL to about 4.5 ng/mL, or from about 3.0 ng/mL to about 4.0 ng/mL, or from about 3.0 ng/mL to about 3.5 ng/mL, or from about 3.5 ng/mL to about 4.5 ng/mL, or from about 3.5 ng/mL to about 4.0 ng/mL, or from about 4.0 ng/mL to about 4.5 ng/mL.

Embodiment 54. The antibody or antigen-binding fragment of any one of Embodiments 1-53, which is capable of neutralizing infection by a H5 pseudovirus with a IC50 of about 0.6 ng/mL, about 0.5 ng/mL, about 0.4 ng/mL, about 0.3 ng/mL, or about 0.2 ng/mL.

Embodiment 55. The antibody or antigen-binding fragment of any one of Embodiments 1-54, which is capable of neutralizing infection by a H5 pseudovirus with a IC50 of 0.7 ng/mL or less, 0.6 ng/mL or less, 0.5 ng/mL or less, 0.4 ng/mL or less, 0.3 ng/mL or less, or 0.20 ng/mL or less.

Embodiment 56. The antibody or antigen-binding fragment of any one of Embodiments 1-55, which is capable of neutralizing infection by: (i) a H1N1 IAV with a IC50 in a range of from about 850 ng/mL to about 4,500 ng/mL, and/or with a IC90 in a range of from about 1,000 ng/mL to about 5,400 ng/mL; and/or (ii) a H3N2 IAV with a IC50 in a range of from about 300 ng/mL to about 2,800 ng/mL, and/or with a IC90 in a range of from about 350 ng/mL to about 7,600 ng/mL.

Embodiment 57. The antibody or antigen-binding fragment of any one of Embodiments 1-56, which is capable of neutralizing infection by: (i) a H1N1 IAV with a IC50 in a range of from about 880 ng/mL to about 1,120 ng/mL, and/or with a IC90 in a range of from about 1,050 ng/mL to about 1,680 ng/mL; (ii) a H3N2 IAV with a IC50 in a range of from about 300 ng/mL to about 2,100 ng/mL and/or with a IC90 in a range of from about 350 ng/mL to about 2,700 ng/mL; (iii) a H1N1 IAV with a IC50 in a range of from about 1,100 ng/mL to about 2,700 ng/mL, and/or with a IC90 in a range of from about 1,040 ng/mL to about 4,540 ng/mL;
(iv) a H3N2 IAV with a IC50 in a range of from about 500 ng/mL to about 2,420 ng/mL and/or with a IC90 in a range of from about 680 ng/mL to about 4,570 ng/mL;
(v) a H1N1 IAV with a IC50 in a range of from about 1,030 ng/mL to about 1,680 ng/mL, and/or with a IC90 in a range of from about 1,780 ng/mL to about 4,760 ng/mL;
(vi) a H3N2 IAV with a IC50 in a range of from about 440 ng/mL to about 2,540 ng/mL and/or with a IC90 in a range of from about 450 ng/mL to about 4,250 ng/mL;
(vii) a H1N1 IAV with a IC50 in a range of from about 1,950 ng/mL to about 2,000 ng/mL, and/or with a IC90 in a range of from about 2,420 ng/mL to about 5,400 ng/mL; and/or (viii) a H3N2 IAV with a IC50 in a range of from about 880 ng/mL to about 2,820 ng/mL and/or with a IC90 in a range of from about 1,170 ng/mL to about 7,630 ng/mL.

Embodiment 58. The antibody or antigen-binding fragment of any one of Embodiments 1-57, which is capable of neutralizing infection by: (i) a H1N1 A/PR/8/34 IAV with a IC50 in a range of from about 850 ng/mL to about 2000 ng/mL (e.g., about 880 ng/mL, about 1,000 ng/mL, about 1100 ng/mL, about 2,000 ng/mL), and/or with a IC90 in a range of from about 1050 ng/mL to about 2,400 ng/mL (e.g., about 1,050 ng/mL, about 1850 ng/mL, about 1,780 ng/mL, about 2,400 ng/mL); (ii) a H1N1 A/Solomon Islands/3/06 IAV with a IC50 in a range of from about 1,100 ng/mL to about 2,700 ng/mL (e.g., about 1,100 ng/mL, about 1,680 ng/mL, about 1950 ng/mL, about 2,700 ng/mL) and/or with a IC90 in a range of from about 1680 ng/mL to about 5,400 ng/mL (e.g., about 1680 ng/mL, about 4,500 ng/mL, about 4700 ng/mL, about 5,400 ng/mL); (iii) a H3N2 A/Aichi/2/68 IAV with a IC50 in a range of from about 2,100 ng/mL to about 2,900 ng/mL (e.g., about 2,100 ng/mL, about 2,400 ng/mL, about 2,500 ng/mL, about 2,800 ng/mL) and/or with a IC90 in a range of from about 2,700 ng/mL to about 7,600 ng/mL (e.g., about 2,700 ng/mL, about 4,200, about 4,500 ng/mL, about 7,600 ng/mL); (iv) a H3N2 A/Brisbane/10/07 IAV with a IC50 in a range of from about 300 ng/mL to about 880 ng/mL (e.g., about 300 ng/mL, about 440 ng/mL, about 500 ng/mL, about 880 ng/mL) and/or with a IC90 in a range of from about 350 ng/mL to about 1,200 ng/mL (e.g., about 350 ng/mL, about 450 ng/mL, about 680 ng/mL, about 1,200 ng/mL); (v) a H1N1 A/CAL/09 IAV with a IC50 in a range of from about 3,100 ng/mL to about 4,500 ng/mL (e.g., about 3,100 ng/mL, about 3,600 ng/mL, about 4,300 ng/mL, about 4,500 ng/mL) and/or with a IC90 in a range of from about 350 ng/mL to about 1,200 ng/mL (e.g., about 350 ng/mL, about 450 ng/mL, about 680 ng/mL, about 1,200 ng/mL); and/or (vi) a H3N2 A/HK/68 IAV with a IC50 in a range of from about 2,000 ng/mL to about 3,000 ng/mL (e.g., about 2,000 ng/mL, about 2,100 ng/mL, about 2,200 ng/mL, about 2,300 ng/mL, about 2,400 ng/mL, about 2,500 ng/mL, about 2,600 ng/mL, about 2,700 ng/mL, about 2,800 ng/mL, about 2,900 ng/mL, about 3,000 ng/mL), preferably in a range of from about 2,100 ng/mL to about 2,500 ng/mL.

Embodiment 59. The antibody or antigen-binding fragment of any one of Embodiments 1-58, which is capable of neutralizing infection by: (i) a H1N1 A/PR/8/34 IAV with a IC50 in a range of: from about 860 to about 920 ng/mL, from about 1,000 to about 1,060 ng/mL, from about 1,080 ng/mL to about 1,140 ng/mL, or from about 1,970 ng/mL to about 2,030 ng/mL, and/or with a IC90 in a range of: from about 1,015 ng/ml to about 1,075 ng/mL, from about 1,750 ng/mL to about 1,810 ng/mL, from about 1,750 ng/mL to about 1,830 ng/mL, or from about 2,390 ng/mL to about 2,450 ng/mL; (ii) a H1N1 A/Solomon Islands/3/06 IAV with a IC50 in a range of from about 1,100 ng/mL to about 2,700 ng/mL (e.g., about 1,100 ng/mL, about 1,680 ng/mL, about 1950 ng/mL, about 2,700 ng/mL) and/or with a IC90 in a range of from about 1680 ng/mL to about 5,400 ng/mL (e.g., about 1680 ng/mL, about 4,500 ng/mL, about 4700 ng/mL, about 5,400 ng/mL); (iii) a H3N2 A/Aichi/2/68 IAV with a IC50 in a range of from about 2,100 ng/mL to about 2,900 ng/mL (e.g., about 2,100 ng/mL, about 2,400 ng/mL, about 2,800 ng/mL) and/or with a IC90 in a range of from about 2,700 ng/mL to about 7,600 ng/mL (e.g., about 2,700 ng/mL, about 4,200, about 4,500 ng/mL, about 7,600 ng/mL); and/or (iv) a H3N3 A/Brisbane/10/07 IAV with a IC50 in a range of from about 300 ng/mL to about 880 ng/mL (e.g., about 300 ng/mL, about 440 ng/mL, about 500 ng/mL, about 88 ng/mL) and/or with a IC90 in a range of from about 350 ng/mL to about 1,200 ng/mL (e.g., about 350 ng/mL, about 450 ng/mL, about 680 ng/mL, about 1,200 ng/mL).

Embodiment 60. The antibody or antigen-binding fragment of any one of Embodiments 1-59, which is capable of binding to any one or more of the following H3N2 IAV subtypes: A/Babol/36/2005; A/Hong Kong/CUHK31987/2011; A/Texas/50/2012; A/Wisconsin/67/2005; A/Netherlands/178/1995; A/Johannesburg/33/1994; A/Guangdong-Luohu/1256/2009; A/California/7/2004; A/Hanoi/EL134/2008; A/Wuhan/359/1995; A/Victoria/210/2009; A/Philippines/472/2002; A/Hanoi/EL201/2009; AVictoria/210/2009; A/Missouri/09/2014; A/Perth/16/2009; A/Wyoming/03/2003; A/Moscow/10/1999; A/Sydney/5/1997; A/Nanchang/933/1995; A/Beijing/32/92; A/Aichi/2/1968; A/Brisbane/10/2007; and A/Switzerland/9715293/2013.

Embodiment 61. The antibody or antigen-binding fragment of Embodiment 60, which is capable of binding to the one or more H3N2 IAV subtype(s) with a logEC50 (ng/mL) in a range of: from about 0.1 to about 6, from about 0.1 to about 5.5, from about 1 to about 5, from about 0.1 to about 4.5, from about 0.1 to about 4.0, from about 0.1 to about 3.5, from about 0.1 to about 3, from about 0.1 to about 2.5, from about 0.1 to about 2.0, from 0.1 to about 1.5, from 0.1 to about 1.0, or of about 1.9, about 1.8, about 1.7, about 1.6, about 1.5, about 1.4, about 1.3, about 1.2, about 1.1, about 1.0, about 0.9, about 0.8, about 0.7, about 0.6, about 0.5, about 0.4, about 0.3, about 0.2, or about 0.1 ng/mL, wherein the binding is as determined by ELISA.

Embodiment 62. The antibody or antigen-binding fragment of any one of Embodiments 1-61, which is capable of binding to one or more of (i)-(iv): (i) a H1 HA, which optionally comprises any one or more of: A/England/195/2009; A/Brisbane/59/2007; A/Solomon Islands/3/2006; A/New Caledonia/20/99; A/Texas/36/1991; A/Taiwan/01/1986; A/New Jersey/8/1976; A/Albany/12/1951; A/Fort Monmouth/1/1947; A/New York/1/1918; A/Puerto Rico/8/34; and A/California/07/2009; (ii) a H2 HA, optionally comprising A/Japan/305/1957; (iii) a H5 HA, optionally comprising A/Vietnam/1194/2004; and (iv) a H9 HA, optionally comprising A/Hong Kong/1073/99.

Embodiment 63. The antibody or antigen-binding fragment of any one of Embodiments 1-62, which binds to H5 HA and/or to H7 HA with a KD of less than 1.0E-12 M, less than 1.0E-11 M, less than 1.0E-10 M, less than 1.0E-9 M, less than 1.0E-8 M, or less than 1.0E-7 M, or of 1.0E-8M or less, of 1.0E-9M or less, of 1.0E-10 or less, of 1.0E-11 or less, or 1.0E-12 or less (e.g., as determined by Bio-Layer Interferometry (BLI)).

Embodiment 64. The antibody or antigen-binding fragment of Embodiment 62, which is capable of binding to one or more of (i)-(iv) with a logEC50 (ng/mL) in a range: from about 0.05 to about 1.5, from about 0.05 to about 1.4, from about 0.05 to about 1.3, from about 0.05 to about 1.2, from about 0.05 to about 1.1, from about 0.05 to about 1.0, from about 0.05 to about 0.9, from about 0.05 to about 0.8, from about 0.05 to about 0.7, from about 0.05 to about 0.6, from about 0.05 to about 0.5, from about 0.1 to about 1, or about 1.3, about 1.2, about 1.1, about 1.0, about 0.9, about 0.8, about 0.7, about 0.6, about 0.5, about 0.4, about 0.3, about 0.2, about 0.1, or about 0.05, wherein the binding is as determined by ELISA.

Embodiment 65. The antibody or antigen-binding fragment of any one of Embodiments 1-64, which is a IgG, IgA, IgM, IgE, or IgD isotype.

Embodiment 66. The antibody or antigen-binding fragment of any one of Embodiments 1-65, which is an IgG isotype selected from IgG1, IgG2, IgG3, and IgG4.

Embodiment 67. The antibody or antigen-binding fragment of any one of Embodiments 1-66, which is human, humanized, or chimeric.

Embodiment 68. The antibody or antigen-binding fragment of any one of Embodiments 1-67, wherein the antibody, or the antigen-binding fragment, comprises a human antibody, a monoclonal antibody, a purified antibody, a single chain antibody, a Fab, a Fab′, a F(ab′)₂, or a Fv, such as a scFv.

Embodiment 69. The antibody or antigen-binding fragment of any one of Embodiments 1-68, wherein the antibody or antigen-binding fragment is a multi-specific antibody or antigen binding fragment.

Embodiment 70. The antibody or antigen-binding fragment of Embodiment 69, wherein the antibody or antigen binding fragment is a bispecific antibody or antigen-binding fragment.

Embodiment 71. The antibody or antigen-binding fragment of any one of Embodiments 1-70, further comprising a Fc polypeptide or a fragment thereof, wherein, optionally, the Fc polypeptide or fragment thereof is an IgG1 isotype.

Embodiment 72. The antibody or antigen-binding fragment of Embodiment 71, wherein the Fc polypeptide or fragment thereof comprises: (i) a mutation that extends in vivo half-life of the antibody or antigen-binding fragment, as compared to the antibody or antigen-binding fragment comprising a reference (e.g., native of a same isotype) Fc polypeptide or fragment thereof that does not comprise the mutation; and/or (ii) a mutation that increases binding affinity to a human FcγR (e.g., a FcγRIIa and/or a FcγRIIIa), as compared to a reference Fc polypeptide that does not comprise the mutation.

Embodiment 73. The antibody or antigen-binding fragment of Embodiment 72, wherein the mutation that extends in vivo half-life of the antibody or antigen-binding fragment comprises: M428L; N434S; N434H; N434A; N434S; M252Y; S254T; T256E; T250Q; P257I; Q311I; D376V; T307A; E380A; or any combination thereof,
wherein Fc amino acid numbering is according to the EU numbering system.

Embodiment 74. The antibody or antigen-binding fragment of Embodiment 72 or 73, wherein the mutation that extends in vivo half-life of the antibody or antigen-binding fragment comprises: (i) M428L/N434S; (ii) M252Y/S254T/T256E;
(iii) T250Q/M428L; (iv) P257I/Q311I; (v) P257I/N434H; (vi) D376V/N434H;
(vii) T307A/E380A/N434A; or (viii) any combination of (i)-(vii).

Embodiment 75. The antibody or antigen-binding fragment of any one of Embodiments 72-74, wherein the mutation that extends in vivo half-life comprises M428L/N434S.

Embodiment 76. The antibody or antigen-binding fragment of any one of Embodiments 72-75, wherein the mutation that enhances binding to a FcγR comprises S239D; 1332E; A330L; G236A; or any combination thereof,
wherein Fc amino acid numbering is according to the EU numbering system.

Embodiment 77. The antibody or antigen-binding fragment of any one of Embodiments 72-76, wherein the mutation that enhances binding to a FcγR comprises: (i) S239D/I332E; (ii) S239D/A330L/I332E; (iii) G236A/S239D/I332E; or (iv) G236A/A330L/I332E, optionally not comprising S239D, further optionally comprising a S at position 239.

Embodiment 78. The antibody or antigen-binding fragment of any one of Embodiments 1-77, wherein the antibody or antigen-binding fragment:
(i) comprises a mutation that alters glycosylation, wherein the mutation that alters glycosylation comprises N297A, N297Q, or N297G; and/or
(ii) is aglycosylated and/or is afucosylated.

Embodiment 79. The antibody or antigen-binding fragment of any one of Embodiments 1-78, comprising a CH1-CH3 that comprises or consists of the amino acid sequence set forth in SEQ ID NO.:47 or 49.

Embodiment 80. The antibody or antigen-binding fragment of any one of Embodiments 1-79, comprising a CL that comprises or consists of the amino acid sequence set forth in SEQ ID NO.:48.

Embodiment 81. An antibody, or an antigen-binding fragment thereof, comprising two heavy chains and two light chains, wherein:
(i) each of the two heavy chains comprises or consists of (1) a heavy chain variable domain (VH), wherein the VH comprises or consists of the amino acid sequence of SEQ ID NO.: 37, and (2) a CH1-CH3 that comprises or consists of the amino acid sequence set forth in SEQ ID NO.:47 or 49; and
(ii) each of the two light chains comprises or consists of (1) a light chain variable domain (VL), wherein the VL comprises or consists of the amino acid sequence of SEQ ID NO.:8, and (2) a CL that comprises or consists of the amino acid sequence of SEQ ID NO.:48.

Embodiment 82. An isolated polynucleotide encoding the antibody or antigen-binding fragment of any one of Embodiments 1-81, or encoding a VH, a heavy chain, a VL, and/or a light chain of the antibody or the antigen-binding fragment.

Embodiment 83. The polynucleotide of Embodiment 82, wherein the polynucleotide comprises deoxyribonucleic acid (DNA) or ribonucleic acid (RNA), wherein the RNA optionally comprises messenger RNA (mRNA).

Embodiment 84. The polynucleotide of Embodiment 82 or 83, comprising a modified nucleoside, a cap-1 structure, a cap-2 structure, or any combination thereof.

Embodiment 85. The polynucleotide of Embodiment 84, wherein the polynucleotide comprises a pseudouridine, a N6-methyladenonsine, a 5-methylcytidine, a 2-thiouridine, or any combination thereof.

Embodiment 86. The polynucleotide of Embodiment 85, wherein the pseudouridine comprises N1-methylpseudouridine.

Embodiment 87. The polynucleotide of any one of Embodiments 82-86, which is codon-optimized for expression in a host cell.

Embodiment 88. The polynucleotide of Embodiment 87, wherein the host cell comprises a human cell.

Embodiment 89. The polynucleotide of any one of Embodiments 82-88, comprising a polynucleotide having at least 50% (e.g., 50%, 55%, 60%, 65%, 70%, 75%, 80%, 85%, 90%, 91%, 92%, 93%, 94%, 95%, 96%, 97%, 98%, 99%, or more) identity to the polynucleotide sequence according to any one or more of SEQ ID NOs.: 1, 6, 7, 12, 25, 27, 30, 33, 36, 13, 18, 19, 24, 38, and 40.

Embodiment 90. The polynucleotide of any one of Embodiments 82-89, comprising: (i) a polynucleotide having at least 75% identity to, or comprising or consisting of, the polynucleotide sequence of SEQ ID NO.:6 and a polynucleotide having at least 75% identity to, or comprising or consisting of, the polynucleotide sequence of SEQ ID NO.:12; (ii) a polynucleotide having at least 75% identity to, or comprising or consisting of, the polynucleotide sequence of SEQ ID NO.:25 and a polynucleotide having at least 75% identity to, or comprising or consisting of, the polynucleotide sequence of SEQ ID NO.:12; (iii) a polynucleotide having at least 75% identity to, or comprising or consisting of, the polynucleotide sequence of SEQ ID NO.:27 and a polynucleotide having at least 75% identity to, or comprising or consisting of, the polynucleotide sequence of SEQ ID NO.:12; (iv) a polynucleotide having at least 75% identity to, or comprising or consisting of, the polynucleotide sequence of SEQ ID NO.:30 and a polynucleotide having at least 75%, at least 80%, at least 85%, at least 90%, at least 91%, at least 92%, at least 93%, at least 94%, at least 95%, at least 96%, at least 97%, at least 98%, or at least 99% identity to, or comprising or consisting of, the polynucleotide sequence of SEQ ID NO.:12; (v) a polynucleotide having at least 75% identity to, or comprising or consisting of, the polynucleotide sequence of SEQ ID NO.:33 and a polynucleotide having at least 75% identity to, or comprising or consisting of, the polynucleotide sequence of SEQ ID NO.:12; (vi) a polynucleotide having at least 75% identity to, or comprising or consisting of, the polynucleotide sequence of SEQ ID NO.:36 and a polynucleotide having at least 75% identity to, or comprising or consisting of, the polynucleotide sequence of SEQ ID NO.:12; (vii) a polynucleotide having at least 75% identity to, or comprising or consisting of, the polynucleotide sequence of SEQ ID NO.:18 and a polynucleotide having at least 75% identity to, or comprising or consisting of, the polynucleotide sequence of SEQ ID NO.:24; (viii) a polynucleotide having at least 75% identity to, or comprising or consisting of, the polynucleotide sequence of SEQ ID NO.:38 and a polynucleotide having at least 75% identity to, or comprising or consisting of, the polynucleotide sequence of SEQ ID NO.:24; or (ix) a polynucleotide having at least 75% identity to, or comprising or consisting of, the polynucleotide sequence of SEQ ID NO.:40 and a polynucleotide having at least 75% identity to, or comprising or consisting of, the polynucleotide sequence of SEQ ID NO.:24.

Embodiment 91. A recombinant vector comprising the polynucleotide of any one of Embodiments 82-90.

Embodiment 92. A host cell comprising the polynucleotide of any one of Embodiments 82-90 and/or the vector of Embodiment 91, wherein the polynucleotide is heterologous to the host cell and wherein the host cell is capable of expressing the encoded antibody or antigen-binding fragment.

Embodiment 93. An isolated human B cell comprising the polynucleotide of any one of Embodiments 82-90 and/or the vector of Embodiment 91, wherein polynucleotide is heterologous to the human B cell and/or wherein the human B cell is immortalized.

Embodiment 94. A composition comprising: (i) the antibody or antigen-binding fragment of any one of Embodiments 1-81; (ii) the polynucleotide of any one of Embodiments 82-90; (iii) the recombinant vector of Embodiment 91;
(iv) the host cell of Embodiment 92; and/or (v) the human B cell of Embodiment 93, and a pharmaceutically acceptable excipient, carrier, or diluent.

Embodiment 95. The composition of Embodiment 94, comprising a first antibody or antigen-binding fragment and a second antibody or antigen-binding fragment, wherein each of the first antibody or antigen-binding fragment and the second antibody or antigen-binding fragment are different and are each independently according any one of Embodiments 1-81.

Embodiment 96. A composition comprising the polynucleotide of any one of Embodiments 82-90 or the vector of Embodiment 91 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

Embodiment 97. A method of making an antibody or antigen-binding fragment of any one of Embodiments 1-81, comprising culturing the host cell of Embodiment 92 or the human B cell of Embodiment 93 for a time and under conditions sufficient for the host cell or human B cell to express the antibody or antigen-binding fragment.

Embodiment 98. The method of Embodiment 97, further comprising isolating the antibody or antigen-binding fragment.

Embodiment 99. A method of treating or preventing an influenza A virus infection in a subject, the method comprising administering to the subject an effective amount of:
(i) the antibody or antigen-binding fragment of any one of Embodiments 1-81;
(ii) the polynucleotide of any one of Embodiments 82-90;
(iii) the recombinant vector of Embodiment 91;
(iv) the host cell of Embodiment 92;
(v) the human B cell of Embodiment 93; and/or
(vi) the composition of any one of Embodiments 94-96.

Embodiment 100. A method of treating or preventing an influenza infection in a human subject, the method comprising administering to the subject the polynucleotide of any one of Embodiments 82-90, the recombinant vector of Embodiment 91, or the composition of Embodiment 96, wherein the polynucleotide comprises mRNA.

Embodiment 101. The method of Embodiment 100, wherein the influenza infection comprises an IAV infection.

Embodiment 102. The method of any one of Embodiments 99-101, comprising administering a single dose of the antibody or antigen-binding fragment, polypeptide, polynucleotide, recombinant vector, host cell, or composition to the subject.

Embodiment 103. The method of any one of Embodiments 99-102, comprising administering two or more doses of the antibody or antigen-binding fragment, polypeptide, polynucleotide, recombinant vector, host cell, or composition to the subject.

Embodiment 104. The method of any one of Embodiments 99-103, comprising administering a dose of the antibody or antigen-binding fragment, polypeptide, polynucleotide, recombinant vector, host cell, or composition to the subject once during a year, optionally in advance of or during an influenza season.

Embodiment 105. The method of any one of Embodiments 99-103, comprising administering a dose of the antibody or antigen-binding fragment, polypeptide, polynucleotide, recombinant vector, host cell, or composition to the subject two or more times during a year; e.g. about once every 6 months.

Embodiment 106. The method of any one of Embodiments 99-105, comprising administering the antibody or antigen-binding fragment, polypeptide, polynucleotide, recombinant vector, host cell, or composition intramuscularly, subcutaneously, or intravenously.

Embodiment 107. The method of any one of Embodiments 99-106, wherein the treatment and/or prevention comprises post-exposure prophylaxis.

Embodiment 108. The method of any one of Embodiments 99-107, wherein the subject has received, is receiving, or will receive an antiviral agent.

Embodiment 109. The method of Embodiment 108, wherein the antiviral agent comprises a neuraminidase inhibitor, an influenza polymerase inhibitor, or both.

Embodiment 110. The method of Embodiment 108 or 109, wherein the antiviral agent comprises oseltamivir, zanamivir, baloxavir, or any combination thereof.

Embodiment 111. The antibody or antigen-binding fragment of any one of Embodiments 1-81, the polynucleotide of any one of Embodiments 82-90, the recombinant vector of Embodiment 91, the host cell of Embodiment 92, the human B cell of Embodiment 93, and/or the composition of any one of Embodiments 94-96, for use in a method of treating or preventing an influenza A virus infection in a subject.

Embodiment 112. The antibody or antigen-binding fragment of any one of Embodiments 1-81, the polynucleotide of any one of Embodiments 82-90, the recombinant vector of Embodiment 91, the host cell of Embodiment 92, the human B cell of Embodiment 93, and/or the composition of any one of Embodiments 94-96, for use in the preparation of a medicament for the treatment of an influenza virus infection in a subject.

Embodiment 113. A method for in vitro diagnosis of an influenza A virus infection, the method comprising:
(i) contacting a sample from a subject with an antibody or antigen-binding fragment of any one of Embodiments 1-81; and
(ii) detecting a complex comprising an antigen and the antibody, or comprising an antigen and the antigen-binding fragment.

TABLE 1
TABLE OF CERTAIN SEQUENCES AND SEQ ID NUMBERS:

| SEQ | | |
|---|---|---|
| ID | | |
| NO | Sequence | Identifier |
| 1 | CAGGTACAACTGCAGCAGTCAGGTCCAGGACTGG | FHF11 VH (wt-nt) |
| | TGAAGCCCTCGCAGACCCTCTCAGTCACCTGTGGC | |
| | ATCTCCGGGGACAGTGTCTCTAGTCACAGTGCT | |
| | GCTTGGAACTGGATCAGGCAGTCCCCATCGAGAG | |
| | GCCTTGAGTGGCTGGGAAGGACATATTACAGGTC | |
| | CAAGTGGTATAATGATTATGCAGTCTCTGTGAAA | |
| | AGTCGAATAACCATCAATCCAGACACATCCAAGA | |
| | ACCAGTTCTCCCTACAGTTGATCTCTGTGACTCCC | |
| | GAGGACACGGCTGTCTATTACTGTGCAAGAGTGG | |
| | GTGCTATGACTTTTGGACTTCTTACAGGGGGTA | |
| | TGGACGTCTGGGGCCAAGGGACCACGGTCACCGT | |
| | CTCCTCA | |
| 2 | QVQLQQSGPGLVKPSQTLSVTCGISGDSVSSHSAAW | FHF11 VH (aa) |
| | NWIRQSPSRGLEWLGRTYYRSKWYNDYAVSVKSRI | |
| | TINPDTSKNQFSLQLISVTPEDTAVYYCARVGAMTF | |
| | GLLTGGMDVWGQGTTVTVSS | |
| 3 | GDSVSSHSAA | FHF11 CDR-H1 (aa) |
| 4 | TYYRSKWYN | FHF11 CDR-H2 (aa) |
| 5 | ARVGAMTFGLLTGGMDV | FHF11 CDR-H3 (aa) |
| 6 | CAGGTGCAGCTGCAGCAGTCTGGACCAGGACTGG | \|FHF11 VH (co-nt) |
| | TGAAGCCTAGCCAGACCCTGTCTGTGACATGCGG | |
| | AATCTCCGGCGACAGCGTGTCCAGCCACTCCGCC | |
| | GCTTGGAACTGGATCAGACAGAGCCCATCTAGGG | |
| | GACTGGAGTGGCTGGGAAGGACCTACTATCGGAG | |
| | CAAGTGGTACAATGACTATGCCGTGTCTGTGAAG | |
| | TCCAGGATCACCATCAACCCAGATACATCCAAGA | |
| | ATCAGTTCAGCCTGCAGCTGATCTCTGTGACCCCC | |
| | GAGGACACAGCCGTGTACTATTGTGCCAGAGTGG | |
| | GCGCTATGACCTTTGGCCTGCTGACAGGCGGAAT | |
| | GGACGTGTGGGGACAGGGAACCACAGTGACAGT | |
| | GTCTTCC | |
| 7 | GAAATTGTGTTGACGCAGTCTCCAGGCACCCAGT | FHF11 Vk (wt-nt) |
| | CTTTGTCTCCAGGGGAAAGAGCCACCCTCTCCTG | |
| | CAGGGCCAGTCAGAGTCTGAGCCGCAGCTACTT | |
| | AGCCTGGTACCAGCAGAGACCTGGCAAGCCTCCC | |
| | AGGCTCCTCATCTATGGTGCATCCAGCAGGGCCA | |
| | CTGGCATCCCAGACAGGTTCAGTGGCAGTGGGTC | |
| | TGGGACAGACTTCAGTCTCACCATCAGCAGTCTG | |
| | GAGCCTGAAGATTCTGCAATGTATTTCTGTCAGT | |
| | ACTATGGTGATTCACCTCTATTCAGTTTCGGCC | |
| | CAGGGACCAAAGTGGATATCAAAC | |
| 8 | EIVLTQSPGTQSLSPGERATLSCRASQSLSRSYLAW | FHF11 Vk (aa) |
| | YQQRPGKPPRLLIYGASSRATGIPDRFSGSGSGTDFS | |
| | LTISSLEPEDSAMYFCQYYGDSPLFSFGPGTKVDIK | |
| 9 | QSLSRSY | FHF11 CDR-L1 (aa) |
| 10 | GAS | FHF11 CDR-L2 (aa) |
| 11 | QYYGDSPLFS | FHF11 CDR-L3 (aa) |
| 12 | GAGATCGTGCTGACCCAGTCTCCTGGCACACAGA | FHF11 Vk (co-nt) |
| | GCCTGTCTCCAGGAGAGAGGGCCACCCTGTCCTG | |
| | CAGGGCTTCCCAGAGCCTGTCTAGGTCCTACCTG | |
| | GCCTGGTATCAGCAGAGACCAGGCAAGCCACCTA | |
| | GGCTGCTGATCTACGGAGCTTCCAGCAGGGCTAC | |
| | AGGCATCCCTGACAGATTCAGCGGCTCTGGCTCC | |
| | GGCACCGATTTTTCCCTGACAATCTCTTCCCTGGA | |
| | GCCAGAGGACTCCGCCATGTATTTCTGTCAGTACT | |
| | ATGGCGATAGCCCACTGTTCTCTTTTGGCCCCGGC | |
| | ACCAAGGTGGACATCAAG | |
| 13 | CAGGTACAACTGCAGCAGTCAGGTCCAGGACTGG | FHF12 VH (wt-nt) |
| | TGAAGCCCTCGCAGACCCTCTCAGTCACCTGTGC | |
| | CATCTCCGGGGACAGTGTCTCTAGTCACAGTGC | |
| | TGCTTGGAACTGGATCAGGCAGTCCCCATCGAGA | |
| | GGCCTTGAGTGGCTGGGAAGGACATATTACAGG | |
| | TCCAAGTGGTATAATGATTATGCAGTCTCTGTGA | |
| | AAAGTCGAATAACCATCAACCCAGACACATCCAA | |
| | GAACCAGTTCTCCCTACAGCTGGTCTCTGTGACTC | |
| | CCGAGGACACGGCTGTCTATTACTGTGCAAGAGT | |
| | GGGTGCTGCGACTTTTGGAATTCTTACAGGGG | |
| | GTATGGACGTCTGGGGCCAAGGGACCACGGTCA | |
| | CCGTCTCCTCA | |
| 14 | QVQLQQSGPGLVKPSQTLSVTCAISGDSVSSHSAA | FHF12 VH (aa) |
| | WNWIRQSPSRGLEWLGRTYYRSKWYNDYAVSVKS | |
| | RITINPDTSKNQFSLQLVSVTPEDTAVYYCARVGAA | |
| | TFGILTGGMDVWGQGTTVTVSS | |
| 15 | GDSVSSHSAA | FHF12 CDR-H1 (aa) |
| 16 | TYYRSKWYN | FHF12 CDR-H2 (aa) |
| 17 | ARVGAATFGILTGGMDV | FHF12 CDR-H3 (aa) |
| 18 | CAGGTGCAGCTGCAGCAGTCTGGACCAGGACTGG | FHF12 VH (co-nt) |
| | TGAAGCCTAGCCAGACCCTGTCTGTGACATGCGCT | |
| | ATCTCCGGCGACAGCGTGTCCAGCCACTCCGCCGC | |
| | TTGGAACTGGATCAGACAGAGCCCATCTAGGGGA | |
| | CTGGAGTGGCTGGGAAGGACCTACTATCGGAGCA | |
| | AGTGGTACAATGACTATGCCGTGTCCGTGAAGTCC | |
| | AGGATCACCATCAACCCAGATACATCCAAGAATC | |
| | AGTTCAGCCTGCAGCTGGTGTCTGTGACCCCCGAG | |
| | GACACAGCCGTGTACTATTGTGCTAGAGTGGGCGC | |
| | CGCTACCTTTGGCATCCTGACAGGCGGAATGGACG | |
| | TGTGGGGACAGGGAACCACAGTGACAGTGTCTTC | |
| | C | |
| 19 | GAAATTGTGTTGACGCAGTCTCCAGGCACCCAGT | FHF12 Vk (wt-nt) |
| | CTTTGTCTCCAGGGGATAGAGCCACCCTCTCCTGC | |
| | AGGGCCAGTCAGAGTCTGAGCAGAAGCTACTTA | |
| | GCCTGGTACCAGCAGAGACCTGGCAAGCCTCCCA | |
| | GGCTCCTCATCTATGGTGCATCCAGCAGGGCCAC | |
| | TGGCATCCCAGACAGGTTCAGTGGCAGTGGGTCT | |
| | GGGACAGACTTCAGTCTCACCATCAGCAGTCTGG | |
| | AGCCTGAAGATTCTGCTATGTATTTCTGTCAGTA | |
| | CTATGGTGATTCACCTCTATTCAGTTTCGGCCC | |
| | TGGGACCAAAGTGGATATCAAAC | |
| 20 | EIVLTQSPGTQSLSPGDRATLSCRASQSLSRSYLAW | FHF 12 Vk (aa) |
| | YQQRPGKPPRLLIYGASSRATGIPDRFSGSGSGTDFS | |
| | LTISSLEPEDSAMYFCQYYGDSPLFSFGPGTKVDIK | |
| 21 | QSLSRSY | FHF12 CDR-L1 (aa) |
| 22 | GAS | FHF12 CDR-L2 (aa) |
| 23 | QYYGDSPLFS | FHF12 CDR-L3 (aa) |
| 24 | GAGATCGTGCTGACCCAGTCTCCTGGCACACAGAGCC | FHF12 Vk (co-nt) |
| | TGTCTCCAGGCGACAGGGCCACCCTGTCCTGCAGGGC | |
| | TTCCCAGAGCCTGTCTAGGTCCTACCTGGCCTGGTATC | |
| | AGCAGAGACCAGGCAAGCCACCTAGGCTGCTGATCTA | |
| | CGGAGCTTCCAGCAGGGCTACAGGCATCCCTGACAGA | |
| | TTCAGCGGCTCTGGCTCCGGCACCGATTTTTCCCTGAC | |
| | AATCTCTTCCCTGGAGCCAGAGGACTCCGCCATGTATT | |
| | TCTGTCAGTACTATGGCGATAGCCCACTGTTCTCTTTT | |
| | GGCCCCGGCACCAAGGTGGATATCAAG | |
| 25 | CAGGTGCAGCTGCAGCAGTCTGGACCAGGACTGG | \|FHF11-VH W36F |
| | TGAAGCCTAGCCAGACCCTGTCTGTGACATGCGGA | (co-nt) |
| | ATCTCCGGCGACAGCGTGTCCAGCCACTCCGCC | |
| | GCTTTCAACTGGATCAGACAGAGCCCATCTAGGG | |
| | GACTGGAGTGGCTGGGAAGGACCTACTATCGGA | |
| | GCAAGTGGTACAATGACTATGCCGTGTCTGTGAA | |
| | GTCCAGGATCACCATCAACCCAGATACATCCAAG | |
| | AATCAGTTCAGCCTGCAGCTGATCTCTGTGACCCC | |
| | CGAGGACACAGCCGTGTACTATTGTGCCAGAGTG | |
| | GGCGCTATGACCTTTGGCCTGCTGACAGGCGG | |
| | AATGGACGTGTGGGGACAGGGAACCACAGTGAC | |
| | AGTGTCTTCC | |
| 26 | QVQLQQSGPGLVKPSQTLSVTCGISGDSVSSHSAAF | FHF11-VH W36F |
| | NWIRQSPSRGLEWLGRTYYRSKWYNDYAVSVKSRI | (aa) |
| | TINPDTSKNQFSLQLISVTPEDTAVYYCARVGAMTF | |
| | GLLTGGMDVWGQGTTVTVSS | |
| 27 | CAGGTGCAGCTGCAGCAGTCTGGACCAGGACTGG | FHF11-VH W59F |
| | TGAAGCCTAGCCAGACCCTGTCTGTGACATGCGGA | (nt) |
| | ATCTCCGGCGACAGCGTGTCCAGCCACTCCGCC | |
| | GCTTGGAACTGGATCAGACAGAGCCCATCTAGGG | |
| | GACTGGAGTGGCTGGGAAGGACCTACTATCGGA | |
| | GCAAGTTCTACAATGACTATGCCGTGTCTGTGAA | |
| | GTCCAGGATCACCATCAACCCAGATACATCCAAG | |
| | AATCAGTTTAGCCTGCAGCTGATCTCTGTGACCCC | |
| | CGAGGACACAGCCGTGTACTATTGTGCCAGAGTG | |
| | GGCGCTATGACCTTCGGCCTGCTGACAGGCGG | |
| | AATGGACGTGTGGGGACAGGGAACCACAGTGAC | |
| | AGTGTCTTCC | |
| 28 | QVQLQQSGPGLVKPSQTLSVTCGISGDSVSSHSAAW | FHF11-VH W59F |
| | NWIRQSPSRGLEWLGRTYYRSKFYNDYAVSVKSRIT | (aa) |
| | INPDTSKNQFSLQLISVTPEDTAVYYCARVGAMTFG | |
| | LLTGGMDVWGQGTTVTVSS | |
| 29 | TYYRSKFYN | FHF11-VH W59F |
| | | CDRH2 (aa) |
| 30 | CAGGTGCAGCTGCAGCAGTCTGGACCAGGACTGG | FHF11v3 VH (co-nt) |
| | TGAAGCCTAGCCAGACCCTGTCTGTGACATGCGG | |
| | CATCTCCGGCGACAGCGTGTCCAGCTACTCCGC | |
| | CGCTTGGAACTGGATCAGACAGAGCCCATCTAGG | |
| | GGACTGGAGTGGCTGGGAAGGACCTACTATCGG | |
| | AGCAAGTGGTACAATGACTATGCCGTGTCTGTG | |
| | AAGTCCAGGATCACCATCAACCCAGATACATCCA | |
| | AGAATCAGTTCAGCCTGCAGCTGATCTCTGTGAC | |
| | CCCCGAGGACACAGCCGTGTACTATTGTGCCAGA | |
| | GTGGGCGCTATGACCTTTGGCCTGCTGACAGG | |
| | CGGAATGGACGTGTGGGGACAGGGAACCACAGT | |
| | GACAGTGTCTTCC | |
| 31 | QVQLQQSGPGLVKPSQTLSVTCGISGDSVSSYSAAW | FHF11v3 VH (aa) |
| | NWIRQSPSRGLEWLGRTYYRSKWYNDYAVSVKSR | |
| | ITINPDTSKNQFSLQLISVTPEDTAVYYCARVGAMT | |
| | FGLLTGGMDVWGQGTTVTVSS | |
| 32 | GDSVSSYSAA | FHF11v3 CDRH1 |
| | | (aa) |
| 33 | CAGGTGCAGCTGCAGCAGTCTGGACCAGGACTGG | FHF11v6 VH (nt) |
| | TGAAGCCTAGCCAGACCCTGTCTGTGACATGCGG | |
| | AATCTCCGGCGACAGCGTGTCCAGCCACTCCG | |
| | CCGCTTGGAACTGGATCAGACAGAGCCCATCTAG | |
| | GGGACTGGAGTGGCTGGGAAGGACCTACTATCG | |
| | GAGCGGCTGGTACAATGACTATGCCGTGTCTGT | |
| | GAAGTCCAGGATCACCATCAACCCAGATACATCC | |
| | AAGAATCAGTTCAGCCTGCAGCTGATCTCTGTGA | |
| | CCCCCGAGGACACAGCCGTGTACTATTGTGCCAG | |
| | AGTGGGCGCTATGACCTTTGGCCTGCTGACAG | |
| | GCGGAATGGACGTGTGGGGACAGGGAACCACA | |
| | GTGACAGTGTCTTCC | |
| 34 | QVQLQQSGPGLVKPSQTLSVTCGISGDSVSSHSAA | FHF11v6 VH (aa) |
| | WNWIRQSPSRGLEWLGRTYYRSGWYNDYAVSVKS | |
| | RITINPDTSKNQFSLQLISVTPEDTAVYYCARVGAM | |
| | TFGLLTGGMDVWGQGTTVTVSS | |
| 35 | TYYRSGWYN | FHF11v6 CDRH2 |
| | | (aa) |
| 36 | CAGGTGCAGCTGCAGCAGTCTGGACCAGGACTGG | FHF11v9 VH (co-nt) |
| | TGAAGCCTAGCCAGACCCTGTCTGTGACATGCGGC | |
| | ATCTCCGGCGACAGCGTGTCCAGCTACTCCGCC | |
| | GCTTGGAACTGGATCAGACAGAGCCCATCTAGGG | |
| | GACTGGAGTGGCTGGGAAGGACCTACTATCGGA | |
| | GCGGCTGGTACAATGACTATGCCGTGTCTGTGAA | |
| | GTCCAGGATCACCATCAACCCAGATACATCCAAG | |
| | AATCAGTTCAGCCTGCAGCTGATCTCTGTGACCCC | |
| | CGAGGACACAGCCGTGTACTATTGTGCCAGAGTG | |
| | GGCGCTATGACCTTTGGCCTGCTGACAGGCGG | |
| | AATGGACGTGTGGGGACAGGGAACCACAGTGAC | |
| | AGTGTCTTCC | |
| 37 | QVQLQQSGPGLVKPSQTLSVTCGISGDSVSSYSAAW | FHF11v9 VH (aa) |
| | NWIRQSPSRGLEWLGRTYYRSGWYNDYAVSVKSR | |
| | ITINPDTSKNQFSLQLISVTPEDTAVYYCARVGAMT | |
| | FGLLTGGMDVWGQGTTVTVSS | |
| 38 | CAGGTGCAGCTGCAGCAGTCTGGACCAGGACTGG | FHF12-VH-W36F |
| | TGAAGCCTAGCCAGACCCTGTCTGTGACATGCGC | (co-nt) |
| | TATCTCCGGCGACAGCGTGTCCAGCCACTCCGC | |
| | CGCTTTCAACTGGATCAGACAGAGCCCATCTAGG | |
| | GGACTGGAGTGGCTGGGAAGGACCTACTATCGG | |
| | AGCAAGTGGTACAATGACTATGCCGTGTCCGTG | |
| | AAGTCCAGGATCACCATCAACCCAGATACATCCA | |
| | AGAATCAGTTCAGCCTGCAGCTGGTGTCTGTGAC | |
| | CCCCGAGGACACAGCCGTGTACTATTGTGCTAGA | |
| | GTGGGCGCCGCTACCTTTGGCATCCTGACAGG | |
| | CGGAATGGACGTGTGGGGACAGGGAACCACAGT | |
| | GACAGTGTCTTCC | |
| 39 | QVQLQQSGPGLVKPSQTLSVTCAISGDSVSSHSAAF | FHF12-VH-W36F |
| | NWIRQSPSRGLEWLGRTYYRSKWYNDYAVSVKSR | (aa) |
| | ITINPDTSKNQFSLQLVSVTPEDTAVYYCARVGAAT | |
| | FGILTGGMDVWGQGTTVTVSS | |
| 40 | CAGGTGCAGCTGCAGCAGTCTGGACCAGGACTGG | FHF12-VH-W59F |
| | TGAAGCCTAGCCAGACCCTGTCTGTGACATGCGC | (co-nt) |
| | TATCTCCGGCGACAGCGTGTCCAGCCACTCCGC | |
| | CGCTTGGAACTGGATCAGACAGAGCCCATCTAGG | |
| | GGACTGGAGTGGCTGGGAAGGACCTACTATCGG | |
| | AGCAAGTTCTACAATGACTATGCCGTGTCCGTGA | |
| | AGTCCAGGATCACCATCAACCCAGATACATCCAA | |
| | GAATCAGTTCAGCCTGCAGCTGGTGTCTGTGACC | |
| | CCCGAGGACACAGCCGTGTACTATTGTGCTAGAG | |
| | TGGGCGCCGCTACCTTTGGCATCCTGACAGGC | |
| | GGAATGGACGTGTGGGGACAGGGAACCACAGTG | |
| | ACAGTGTCTTCC | |
| 41 | QVQLQQSGPGLVKPSQTLSVTCAISGDSVSSHSAA | FHF12-VH-W59F |
| | WNWIRQSPSRGLEWLGRTYYRSKFYNDYAVSVKS | (aa) |
| | RITINPDTSKNQFSLQLVSVTPEDTAVYYCARVGAA | |
| | TFGILTGGMDVWGQGTTVTVSS | |
| 42 | TYYRSKFYN | FHF12-CDRH2- |
| | | W59F (aa) |
| 43 | QVQLQQSGPGLVKPSQTLSLTCAISGDSVSSYNAVW | FM08 VH |
| | NWIRQSPSRGLEWLGRTYYRSGWYNDYAESVKSRI | |
| | TINPDTSKNQFSLQLNSVTPEDTAVYYCARSGHITVF | |
| | GVNVDAFDMWGQGTMVTVSS | |
| 44 | DIQMTQSPSSLSASVGDRVTITCRTSQSLSSYTHWY | FM08 VL |
| | QQKPGKAPKLLIYAASSRGSGVPSRFSGSGSGTDFT | |
| | LTISSLQPEDFATYYCQQSRTFGQGTKVEIK | |
| 45 | APELLGGPSVFLFPPKPKDTLMISRTPEVTCVVVDVS | WT hIgG1 Fc |
| | HEDPEVKFNWYVDGVEVHNAKTKPREEQYNSTYR | |
| | VVSVLTVLHQDWLNGKEYKCKVSNKALPAPIEKTI | |
| | SKAKGQPREPQVYTLPPSRDELTKNQVSLTCLVKGF | |
| | YPSDIAVEWESNGQPENNYKTTPPVLDSDGSFFLYS | |
| | KLTVDKSRWQQGNVFSCSVMHEALHNHYTQKSLS | |
| | LSPGK | |
| 46 | ESKYGPPCPPCPAPPVAGP | Chimeric hinge |
| | | sequence |
| 47 | ASTKGPSVFPLAPSSKSTSGGTAALGCLVKDYFPEP | IgHG1*01, G1m3 |
| | VTVSWNSGALTSGVHTFPAVLQSSGLYSLSSVVTVP | CH1-CH3 with |
| | SSSLGTQTYICNVNHKPSNTKVDKRVEPKSCDKTHT | M428L and N434S |
| | CPPCPAPELLGGPSVFLFPPKPKDTLMISRTPEVTCV | mutations and C- |
| | VVDVSHEDPEVKFNWYVDGVEVHNAKTKPREEQY | terminal lysine |
| | NSTYRVVSVLTVLHQDWLNGKEYKCKVSNKALPA | |
| | PIEKTISKAKGQPREPQVYTLPPSREEMTKNQVSLTC | |
| | LVKGFYPSDIAVEWESNGQPENNYKTTPPVLDSDGS | |
| | FFLYSKLTVDKSRWQQGNVFSCSVLHEALHSHYTQ | |
| | KSLSLSPGK | |
| 48 | RTVAAPSVFIFPPSDEQLKSGTASVVCLLNNFYPREA | Kappa light chain |
| | KVQWKVDNALQSGNSQESVTEQDSKDSTYSLSSTL | CL |
| | TLSKADYEKHKVYACEVTHQGLSSPVTKSFNRGEC | |
| 49 | ASTKGPSVFPLAPSSKSTSGGTAALGCLVKDYFPEP | IgHG1*01, G1m3 |
| | VTVSWNSGALTSGVHTFPAVLQSSGLYSLSSVVTVP | CH1-CH3 with |
| | SSSLGTQTYICNVNHKPSNTKVDKRVEPKSCDKTHT | M428L and N434S |
| | CPPCPAPELLGGPSVFLFPPKPKDTLMISRTPEVTCV | mutations, without |
| | VVDVSHEDPEVKFNWYVDGVEVHNAKTKPREEQY | C-terminal lysine |
| | NSTYRVVSVLTVLHQDWLNGKEYKCKVSNKALPA | |
| | PIEKTISKAKGQPREPQVYTLPPSREEMTKNQVSLTC | |
| | LVKGFYPSDIAVEWESNGQPENNYKTTPPVLDSDGS | |
| | FFLYSKLTVDKSRWQQGNVFSCSVLHEALHSHYTQ | |
| | KSLSLSPG | |
| 50 | QVQLQQSGPGLVKPSQTLSVTCGISGDSVSSYSAAW | FHF11v9-MLNS |
| | NWIRQSPSRGLEWLGRTYYRSGWYNDYAVSVKSRI | heavy chain with |
| | TINPDTSKNQFSLQLISVTPEDTAVYYCARVGAMTF | C-terminal lysine |
| | GLLTGGMDVWGQGTTVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNVNH | |
| | KPSNTKVDKRVEPKSCDKTHTCPPCPAPELLGGPSV | |
| | FLFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPAPIEKTISKAKGQPRE | |
| | PQVYTLPPSREEMTKNQVSLTCLVKGFYPSDIAVEW | |
| | ESNGQPENNYKTTPPVLDSDGSFFLYSKLTVDKSRW | |
| | QQGNVFSCSVLHEALHSHYTQKSLSLSPGK | |
| 51 | QVQLQQSGPGLVKPSQTLSVTCGISGDSVSSYSAAW | FHF11v9-MLNS |
| | NWIRQSPSRGLEWLGRTYYRSGWYNDYAVSVKSRI | heavy chain |
| | TINPDTSKNQFSLQLISVTPEDTAVYYCARVGAMTF | without C-terminal |
| | GLLTGGMDVWGQGTTVTVSSASTKGPSVFPLAPSS | lysine |
| | KSTSGGTAALGCLVKDYFPEPVTVSWNSGALTSGV | |
| | HTFPAVLQSSGLYSLSSVVTVPSSSLGTQTYICNVNH | |
| | KPSNTKVDKRVEPKSCDKTHTCPPCPAPELLGGPSV | |
| | FLFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPAPIEKTISKAKGQPRE | |
| | PQVYTLPPSREEMTKNQVSLTCLVKGFYPSDIAVEW | |
| | ESNGQPENNYKTTPPVLDSDGSFFLYSKLTVDKSRW | |
| | QQGNVFSCSVLHEALHSHYTQKSLSLSPG | |
| 52 | EIVLTQSPGTQSLSPGERATLSCRASQSLSRSYLAWY | FHF11v9-MLNS |
| | QQRPGKPPRLLIYGASSRATGIPDRFSGSGSGTDFSL | light chain |
| | TISSLEPEDSAMYFCQYYGDSPLFSFGPGTKVDIKRT | |
| | VAAPSVFIFPPSDEQLKSGTASVVCLLNNFYPREAK | |
| | VQWKVDNALQSGNSQESVTEQDSKDSTYSLSSTLT | |
| | LSKADYEKHKVYACEVTHQGLSSPVTKSFNRGEC | |
| 53 | GDSVSSHSAAF | FHF11 and FHF12 |
| | | CDRH1 with |
| | | flanking Phe (aa) |
| 54 | CAGGTGCAGCTGCAGCAGTCTGGACCAGGACTGGTGAAGCC | FHF11v9 heavy |
| | TAGCCAGACCCTGTCTGTGACATGCGGCATCTCCGGCGACA | chain nucleotide |
| | GCGTGTCCAGCTACTCCGCCGCTTGGAACTGGATCAGACAG | sequence |
| | AGCCCATCTAGGGGACTGGAGTGGCTGGGAAGGACCTACTA | |
| | TCGGAGCGGCTGGTACAATGACTATGCCGTGTCTGTGAAGT | |
| | CCAGGATCACCATCAACCCAGATACATCCAAGAATCAGTTC | |
| | AGCCTGCAGCTGATCTCTGTGACCCCCGAGGACACAGCCGT | |
| | GTACTATTGTGCCAGAGTGGGCGCTATGACCTTTGGCCTGCT | |
| | GACAGGCGGAATGGACGTGTGGGGACAGGGAACCACAGTG | |
| | ACAGTGTCTTCCGCATCGACCAAGGGCCCATCGGTCTTCCC | |
| | CCTGGCACCAAGTAGCAAGAGCACATCCGGTGGCACAGCC | |
| | GCCCTGGGTTGTCTGGTGAAAGATTATTTCCCTGAGCCCGTG | |
| | ACAGTCTCCTGGAACTCTGGCGCCCTGACCTCCGGAGTGCA | |
| | CACATTCCCTGCTGTGCTGCAGTCCAGCGGCCTGTACTCCCT | |
| | GTCTTCCGTGGTGACCGTGCCAAGCTCTTCCCTGGGCACCCA | |
| | GACATATATCTGCAACGTGAATCACAAGCCTTCCAATACAA | |
| | AGGTGGACAAGAGGGTGGAGCCAAAGAGCTGTGATAAGAC | |
| | CCATACATGCCCACCTTGTCCAGCTCCAGAGCTGCTGGGCG | |
| | GCCCATCCGTGTTCCTGTTTCCACCCAAGCCCAAGGACACC | |
| | CTGATGATCTCTAGAACCCCAGAGGTGACATGCGTGGTGGT | |
| | GGACGTGTCCCACGAGGATCCCGAGGTGAAGTTTAACTGGT | |
| | ACGTGGATGGCGTGGAGGTGCATAATGCTAAGACAAAGCC | |
| | CAGGGAGGAGCAGTACAACAGCACCTATCGGGTGGTGTCTG | |
| | TGCTGACAGTGCTGCATCAGGACTGGCTGAACGGCAAGGAG | |
| | TATAAGTGCAAGGTGAGCAATAAGGCCCTGCCTGCTCCAAT | |
| | CGAGAAGACCATCTCTAAGGCCAAGGGCCAGCCCAGAGAG | |
| | CCTCAGGTGTACACACTGCCTCCAAGCCGCGAGGAGATGAC | |
| | CAAGAACCAGGTGTCTCTGACATGTCTGGTGAAGGGCTTCT | |
| | ATCCCTCTGACATCGCTGTGGAGTGGGAGTCCAATGGCCAG | |
| | CCTGAGAACAATTACAAGACCACACCCCCTGTGCTGGACTC | |
| | CGATGGCAGCTTCTTTCTGTATTCCAAGCTGACCGTGGATAA | |
| | GAGCAGGTGGCAGCAGGGCAACGTGTTCTCCTGTTCTGTGA | |
| | TGCACGAAGCCCTGCACAACCATTATACTCAGAAGTCCCTG | |
| | TCCCTGTCCCCTGGAAAA | |
| 55 | GAAATTGTGTTGACGCAGTCTCCAGGCACCCAGTCTTTGTCT | FHF11v9 light chain |
| | CCAGGGGAAAGAGCCACCCTCTCCTGCAGGGCCAGTCAGA | nucleotide sequence |
| | GTCTGAGCCGCAGCTACTTAGCCTGGTACCAGCAGAGACCT | |
| | GGCAAGCCTCCCAGGCTCCTCATCTATGGTGCATCCAGCAG | |
| | GGCCACTGGCATCCCAGACAGGTTCAGTGGCAGTGGGTCTG | |
| | GGACAGACTTCAGTCTCACCATCAGCAGTCTGGAGCCTGAA | |
| | GATTCTGCAATGTATTTCTGTCAGTACTATGGTGATTCACCT | |
| | CTATTCAGTTTCGGCCCAGGGACCAAAGTGGATATCAAACC | |
| | GTACGGTGGCTGCACCATCTGTCTTCATCTTCCCGCCATCTG | |
| | ATGAGCAaTTGAAATCTGGAACTGCCTCCGTGGTGTGCCTGC | |
| | TGAACAATTTCTACCCCAGGGAGGCCAAGGTGCAGTGGAAG | |
| | GTGGACAACGCTCTGCAGAGCGGCAATTCTCAGGAGTCCGT | |
| | GACCGAGCAGGACAGCAAGGATTCTACATATTCCCTGTCCA | |
| | GCACCCTGACACTGAGCAAGGCCGATTACGAGAAGCACAA | |
| | GGTGTATGCTTGTGAGGTGACCCATCAGGGCCTGTCTTCCCC | |
| | TGTGACAAAGTCTTTCAACAGGGGAGAGTGT | |
| 56 | QVQLQQSGPGLVKPSQTLSVTCGISGDSVSSYSAAW | FHF11v9 heavy |
| | NWIRQSPSRGLEWLGRTYYRSGWYNDYAVSVKSRI | chain (aa) |
| | TINPDTSKNQFSLQLISVTPEDTAVYYCARVGAMTF | |
| | GLLTGGMDVWGQGTTVTVSSASTKGPSVFPLAPSS | |
| | KSTSGGTAALGCLVKDYFPEPVTVSWNSGALTSGV | |
| | HTFPAVLQSSGLYSLSSVVTVPSSSLGTQTYICNVNH | |
| | KPSNTKVDKRVEPKSCDKTHTCPPCPAPELLGGPSV | |
| | FLFPPKPKDTLMISRTPEVTCVVVDVSHEDPEVKFN | |
| | WYVDGVEVHNAKTKPREEQYNSTYRVVSVLTVLH | |
| | QDWLNGKEYKCKVSNKALPAPIEKTISKAKGQPRE | |
| | PQVYTLPPSREEMTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| 57 | QVQLQQSGPGLVKPSQTLSLTCAISGDSVSSNSAAW | FHF-VH-UCA |
| | NWIRQSPSRGLEWLGRTYYRSKWYNDYAVSVKSRI | |
| | TINPDTSKNQFSLQLNSVTPEDTAVYYCARVGAMIF | |
| | GLLTGGMDVWGQGTTVTVSS | |
| 58 | EIVLTQSPGTLSLSPGERATLSCRASQSVSSSYLAWY | FHF-VL-UCA |
| | QQKPGQAPRLLIYGASSRATGIPDRFSGSGSGTDFTL | |
| | TISRLEPEDFAVYYCQQYGSSPLFTFGPGTKVDIK | |

TABLE 2
Sequence Key - SEQ ID NOs. for certain antibodies

| | SEQ ID NO.: | | | | | | | | | | | |
|---|---|---|---|---|---|---|---|---|---|---|---|---|
| | | | | | | | VL | | | | | |
| | VH | | CDR | CDR | CDR | | (Vk) | VL | CDR | CD | CD | VL |
| | (wt- | VH | H1 | H2 | H3 | VH | wt- | (Vk) | L1 | RL2 | RL3 | (Vk) |
| Antibody | nt) | (aa) | (aa) | (aa) | (aa) | (co-nt) | nt | (aa) | (aa) | (aa) | (aa) | (co-nt) |
| FHF11 | 1 | 2 | 3 | 4 | 5 | 6 | 7 | 8 | 9 | 10 | 11 | 12 |
| FHF11- | | 26 | 3 | 4 | 5 | 25 | | 8 | 9 | 10 | 11 | 12 |
| VH | | | | | | | | | | | | |
| W36F | | | | | | | | | | | | |
| FHF11- | | 28 | 3 | 29 | 5 | 27 | | 8 | 9 | 10 | 11 | 12 |
| VH | | | | | | | | | | | | |
| W59F | | | | | | | | | | | | |
| FHF11 | | 31 | 32 | 4 | 5 | 30 | | 8 | 9 | 10 | 11 | 12 |
| v3 | | | | | | | | | | | | |
| FHF11 | | 34 | 3 | 35 | 5 | 33 | | 8 | 9 | 10 | 11 | 12 |
| v6 | | | | | | | | | | | | |
| FHF11 | | 37 | 32 | 35 | 5 | 36 | | 8 | 9 | 10 | 11 | 12 |
| v9 | | | | | | | | | | | | |
| FHF12 | 13 | 14 | 15 | 16 | 17 | 18 | 19 | 20 | 21 | 22 | 23 | 24 |
| FHF12- | | 39 | 15 | 16 | 17 | 38 | | 20 | 21 | 22 | 23 | 24 |
| VH- | | | | | | | | | | | | |
| W36F | | | | | | | | | | | | |
| FHF12- | | 41 | 15 | 42 | 17 | 40 | | 20 | 21 | 22 | 23 | 24 |
| VH- | | | | | | | | | | | | |
| W59F | | | | | | | | | | | | |

### EXAMPLES

### Example 1

### Identification and Functional Testing of Anti-HA Monoclonal Antibodies

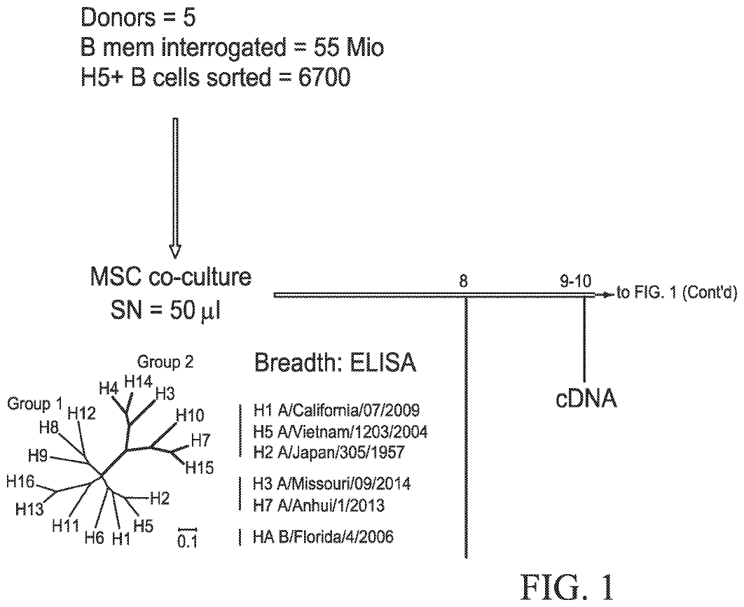

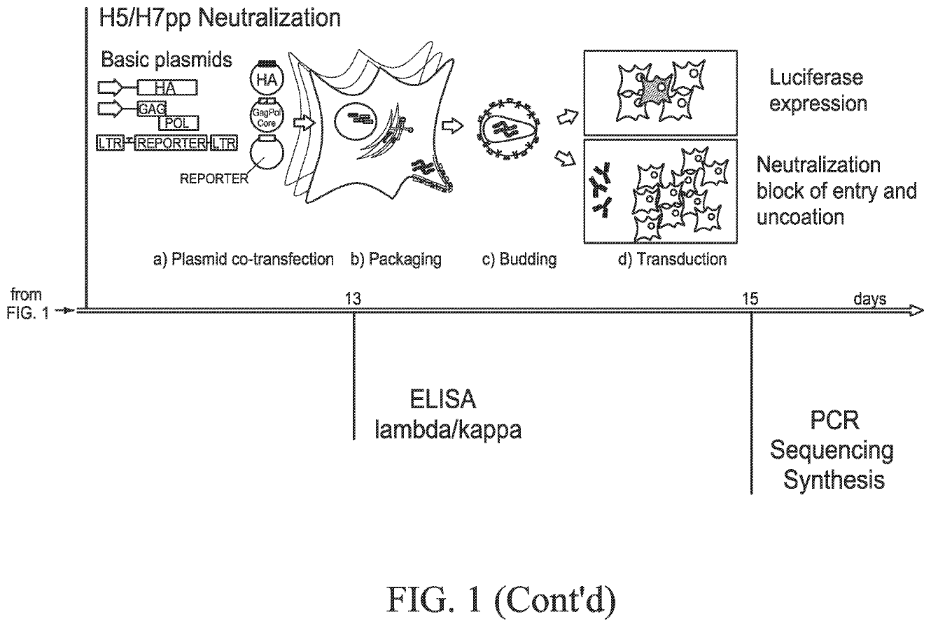

Peripheral blood mononuclear cells (PBMCs) from anonymous donors were selected based on neutralization by the corresponding serum against H5 (Group 1) and H7 (Group 2) influenza pseudoviruses. Donors were selected by screening serum from tonsillar donor samples (n=50) for reactivity against hemagglutinin subtype H5 and H7 antigens, and serum from PBMC donor samples (n=124) for reactivity against H5 and H7 subtype pseudoviruses. Binding was evaluated by FACS. B memory cells from five donors were sorted by flow cytometry for input into the discovery workflow (FIG. 1). Single sorted B cells (n=6,700) were co-cultured with mesenchymal stromal cells (MSC) in 50 μl culture to stimulate antibody secretion. Secreted antibodies were evaluated using binding and pseudovirus neutralization assays. Binding to HAs from group I influenza A viruses (IAV), group II IAVs, and influenza B viruses was evaluated by enzyme-linked immunosorbent assay (ELISA) to determine breadth.

Neutralization—measured as blockade of viral entry and uncoating—was evaluated by monitoring luciferase expression following infection of target cells with H5 or H7 luciferase (Luc)-expressing pseudovirus particles. Antibody sequences from selected B cells were cloned as cDNAs and sequenced.

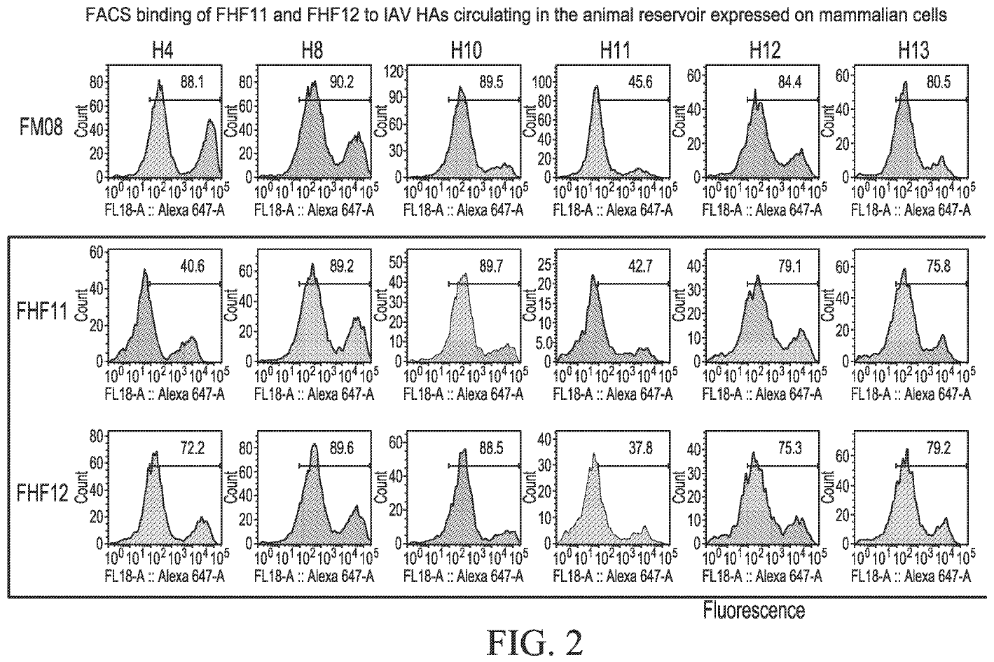

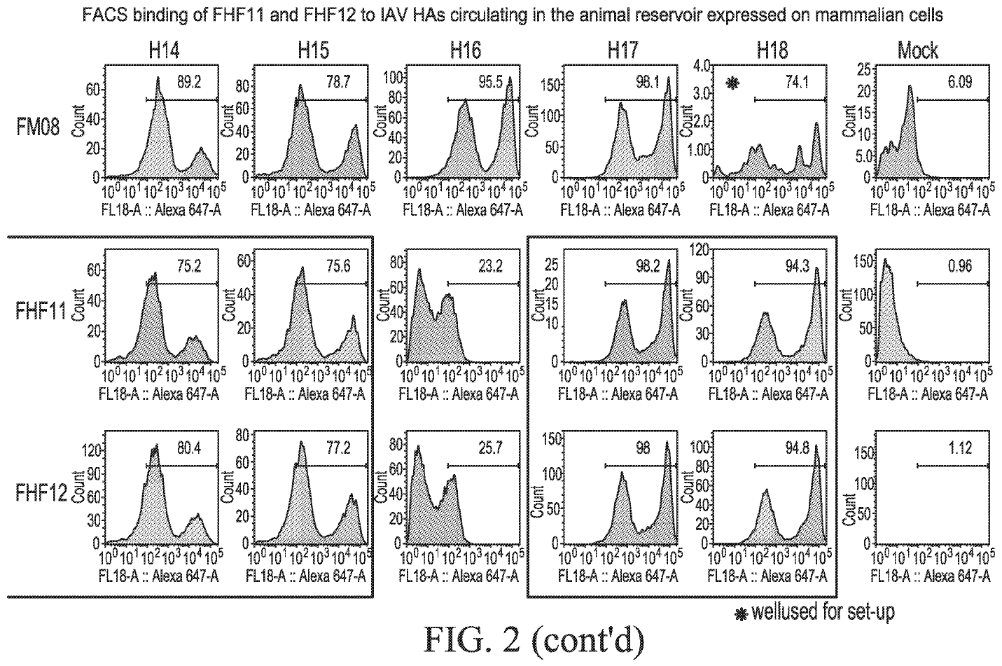

Two clonally related monoclonal antibodies, FHF11 (VH: SEQ ID NO.:2; VL: SEQ ID NO.:8) and FHF12 (VH: SEQ ID NO.:14; VL: SEQ ID NO.:20), were selected for further study. Binding of these antibodies to influenza A virus (IAV)-derived hemagglutinin (HA) was evaluated using FACS; in this assay, IAV-derived HAs circulating in the animal reservoir were expressed on mammalian cells and antibody binding was measured, along with that of comparator antibody FM08 (VH: SEQ ID NO.:43; VL: SEQ ID NO.:44; see also MEDI8852 (Kallewaard et al., Cell 166(3):596-608 (2016), in particular FIG. 1 therein). Data are shown in FIG. 2.

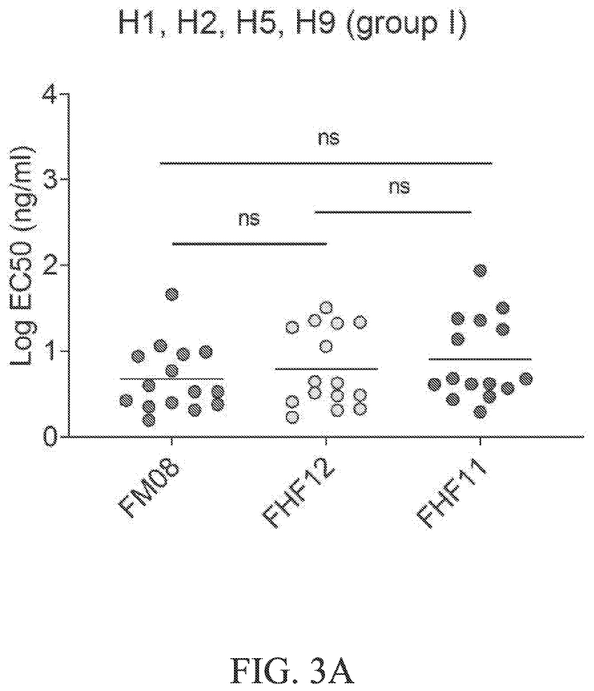

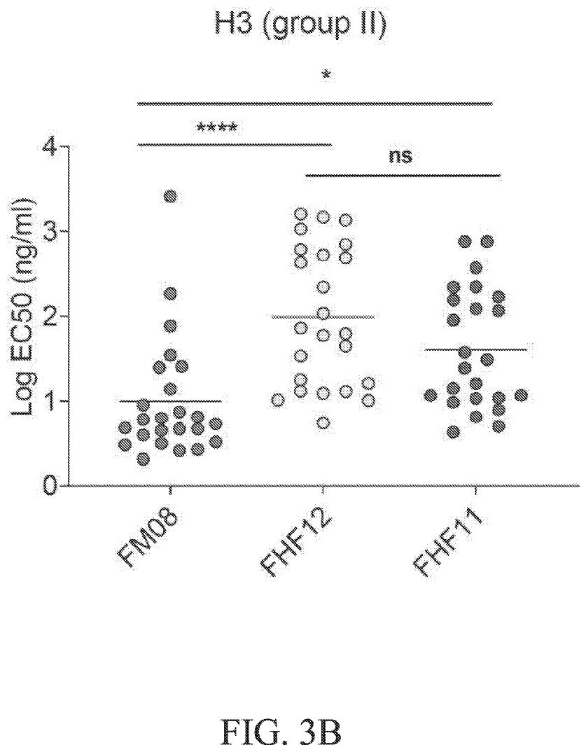

Binding of FHF11 and FHF12 to group I IAV-derived H1, H2, H5, and H9 (FIG. 3A) and group II IAV-derived H3 (FIG. 3B) were measured by ELISA, reported as Log EC50 (ng/ml). Binding by FM08, was also measured.

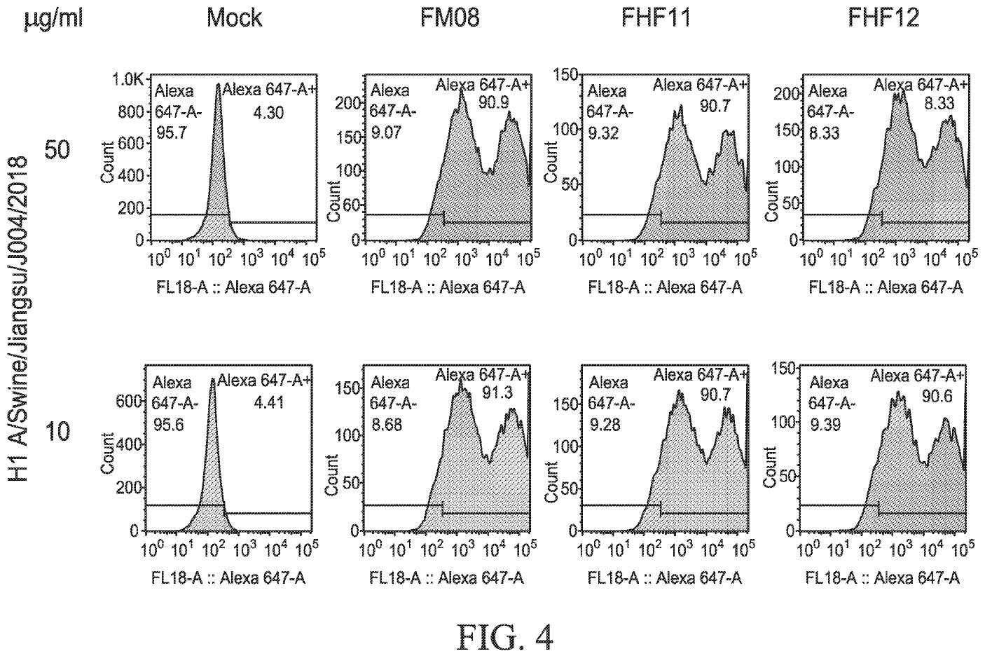

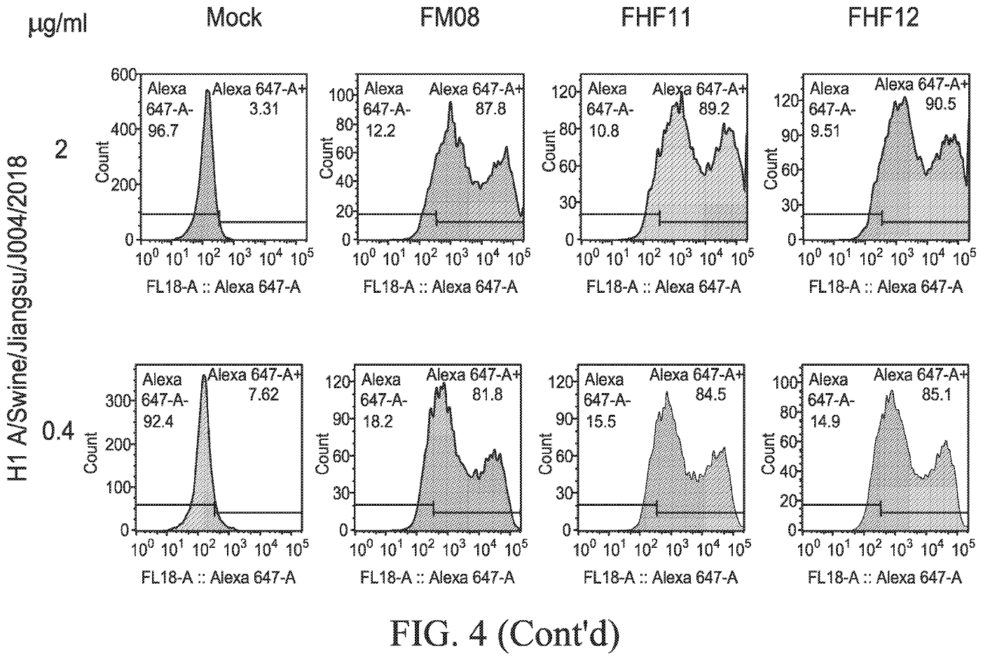

Binding of FHF11 and FHF12 to HA from an H1N1 Swine Eurasian avian-like (EA) strain, A/Swine/Jiangsu/J004/2018, expressed in mammalian cells, was also measured by flow cytometry (FIG. 4).

Polyreactivity, or whether FHF11 and FHF12 bind non-specifically to unrelated self and/or foreign targets, was evaluated using human epithelial type 2 (HEP-2) cells (FIG. 5). A polyreactive antibody, FI6v3.11.18, was included as a positive control, and anti-paramyxovirus antibody “MPE8” (Corti et al. Nature 501(7467):439-43 (2013)) was included as a negative control.

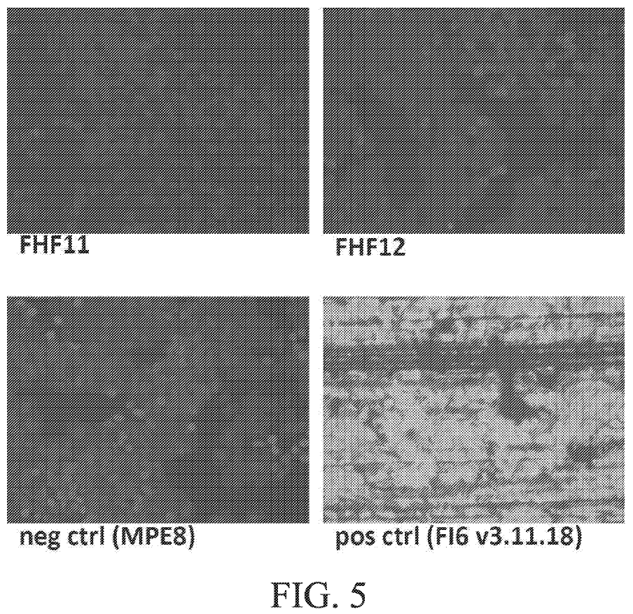

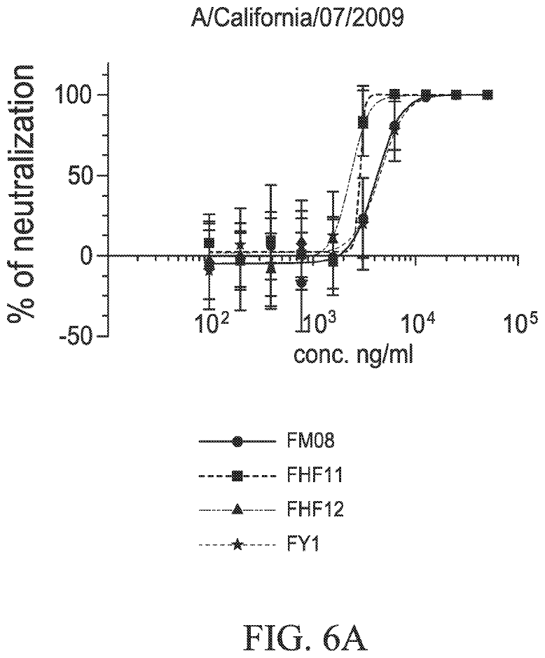

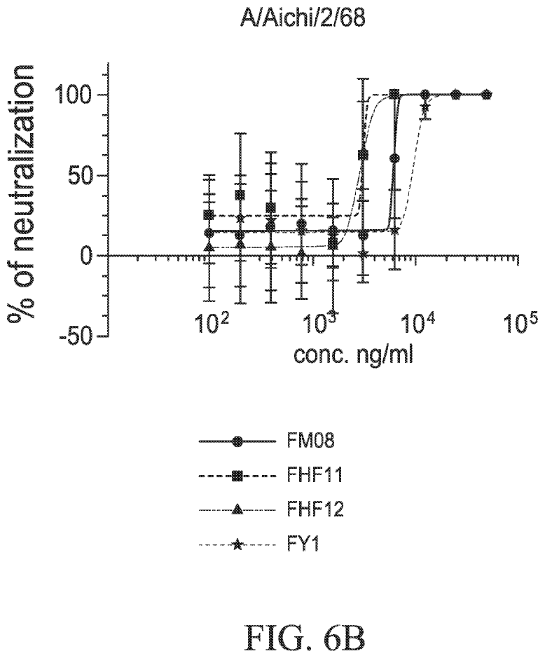

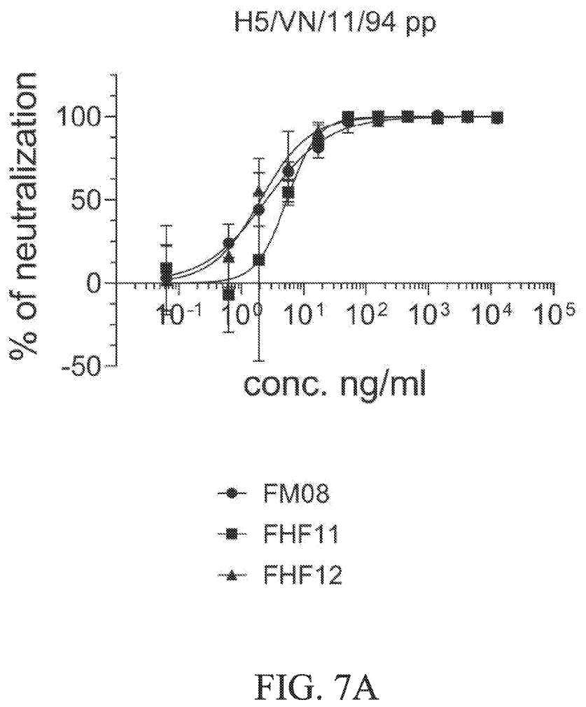

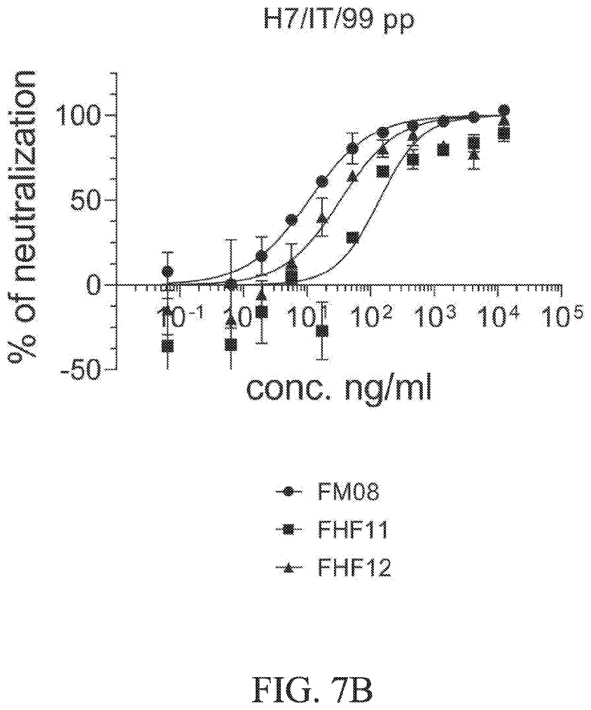

Neutralization by FHF11 and FHF12 against H1N1 (group I) IAV strains A/California/09 (FIG. 6A) and H3N2 (group II) A/Aichi/2/68 (FIG. 6B) pseudovirus was evaluated in in vitro studies. Comparator antibodies FM08 and FY1 (FY1 is also described in Kaaleward et al., Cell 166(3):596-608 (2018); e.g., FIG. 1 therein) were also assessed. FHF11 and FHF12, along with FM08, were further assessed for neutralizing ability against H5 (H5/VN/11/94 pp; FIG. 7A) and H7 (H7/IT/99 pp; FIG. 7B) pseudotyped viruses.

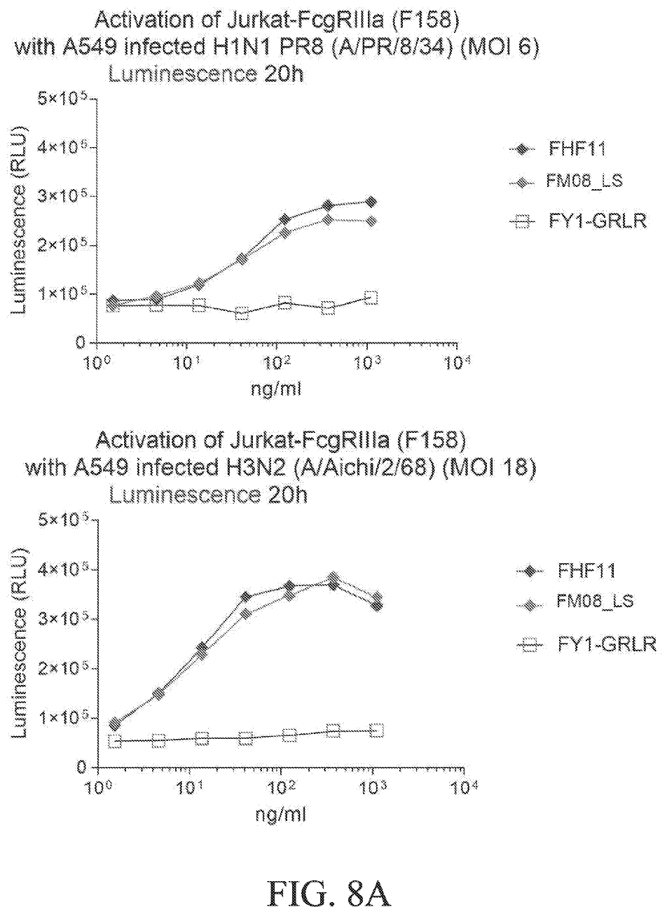

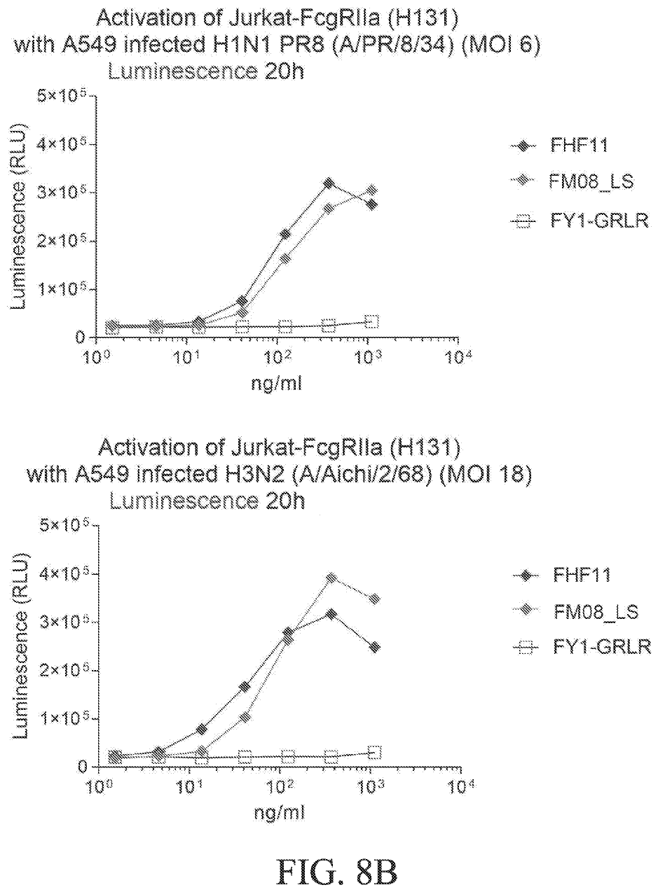

FHF11 activation of FcγRIIIa (FIG. 8A) and FcγRIIa (FIG. 8B) variants was evaluated using a NFAT-driven luciferase reporter assay. Activation of Jurkat-FcγRIIIa (F158) and Jurkat-FcγRIIa (H131) cell lines was assessed following a 20 hour incubation with A549 cells infected with H1N1 influenza strain A/Puerto Rico/8/1934 at a MOI of 6 and with H3N2 influenza strain A/Aichi/2/1968 at a MOI of 18. FM08 antibody comprising a MLNS (M428L/N434S; “LS” in the figure) Fc mutation was used as a comparator, and FY1 antibody comprising a GRLR (G236R/L328R) Fc mutation used as a reference.

### Example 2

### Engineering and Testing Anti-Ha Antibody Variants

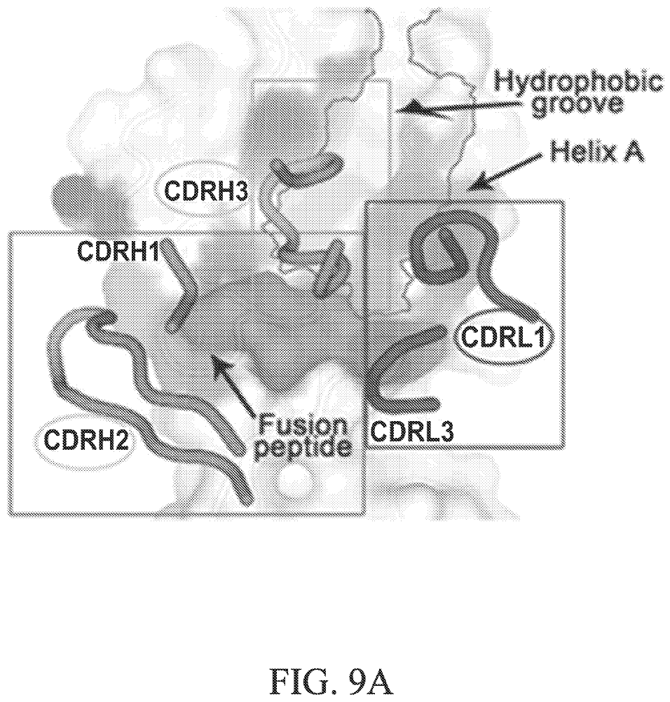

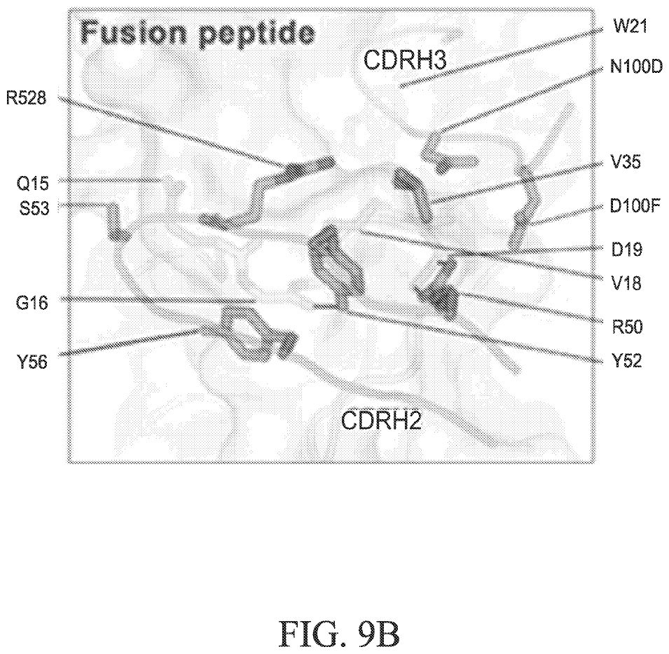

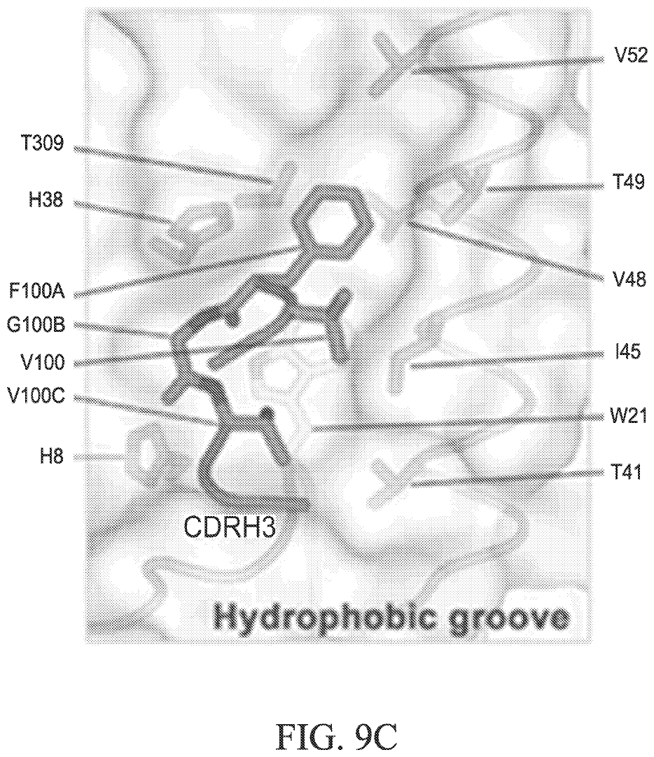

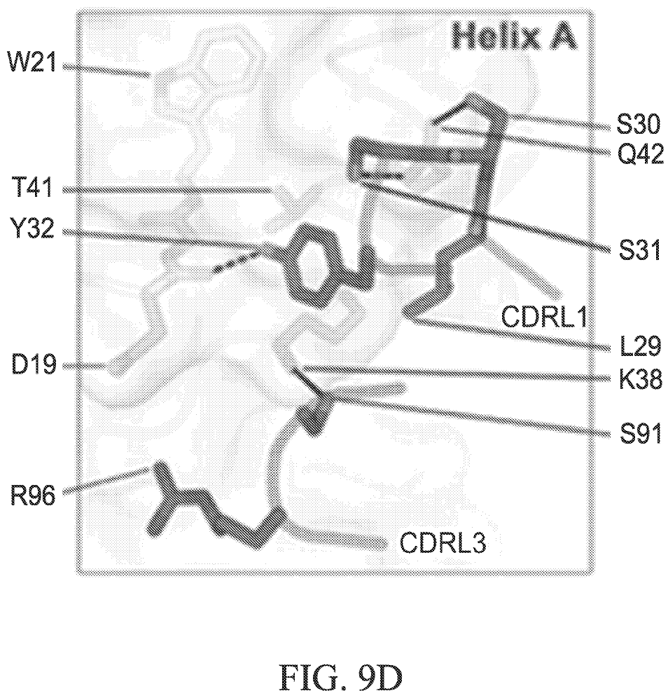

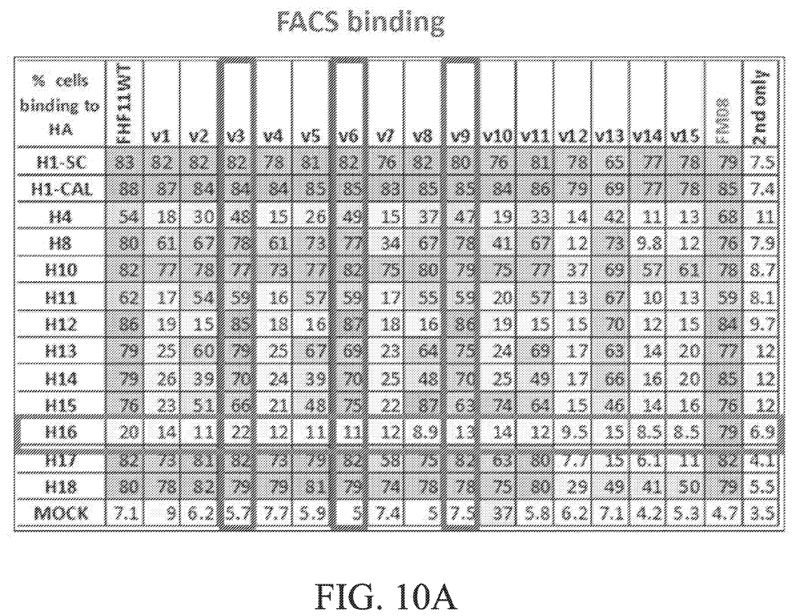

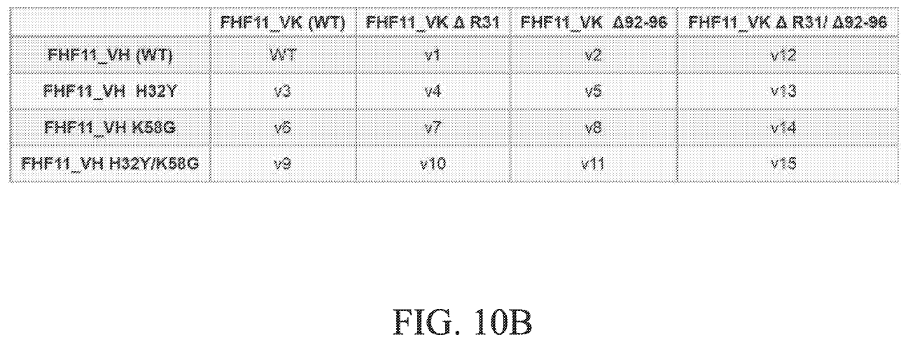

FHF11 was found to use VH6-1/DH3-3 genes. FIGS. 9A-9D illustrate binding interactions between FM08, which utilizes these same genes, and IAV HA. Fifteen (15) variants of FHF11 were generated by engineering in one or both of the variable domains. A summary of sequence differences between FHF11-WT and each of the variant antibodies (v1 to v15) is shown in FIG. 10B. These antibodies were tested for binding to HA and neutralization of infection.

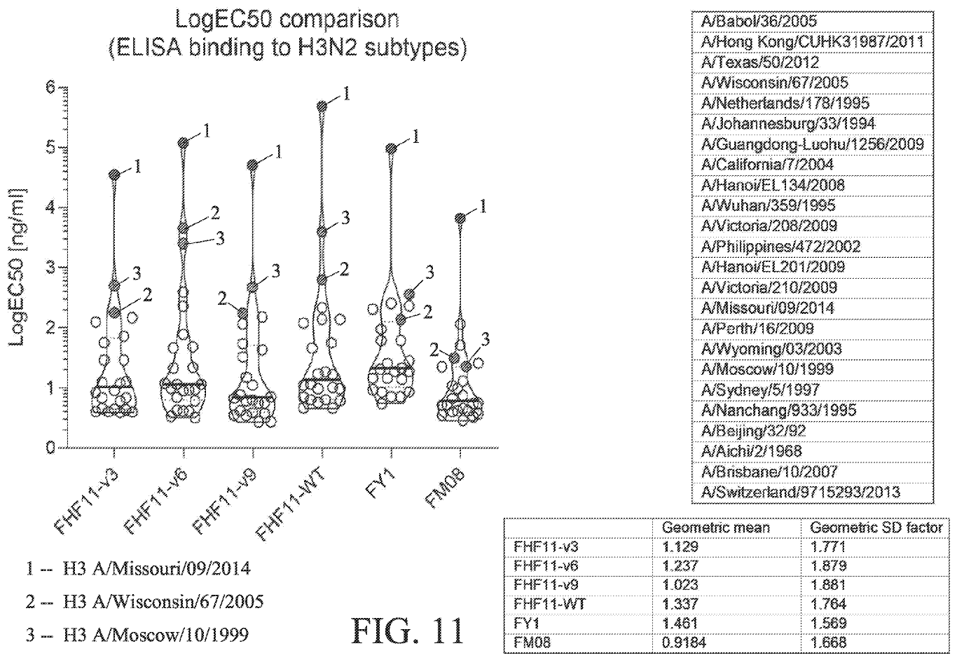

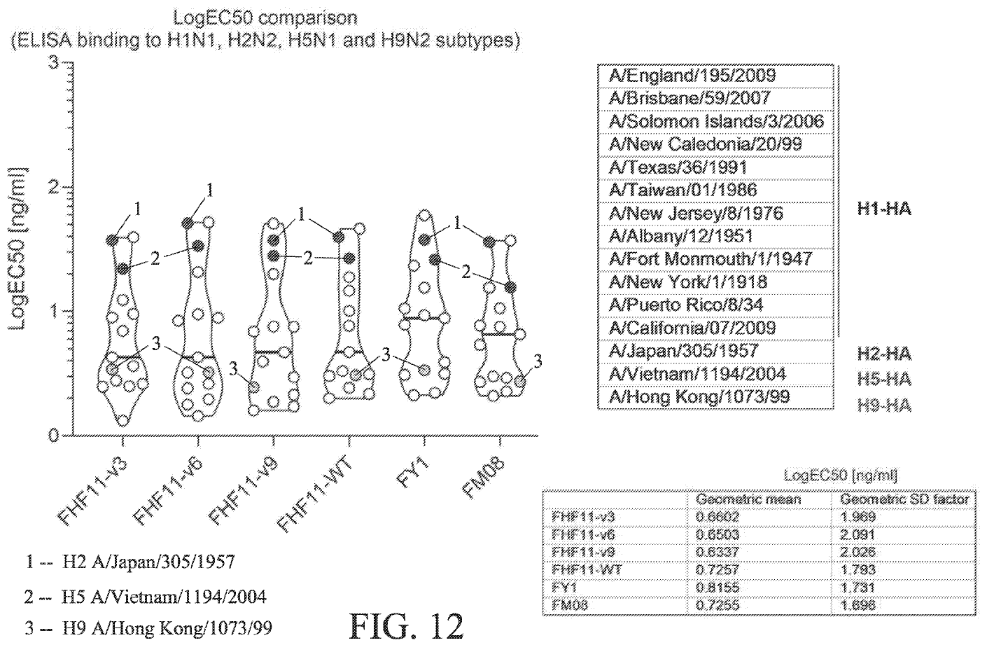

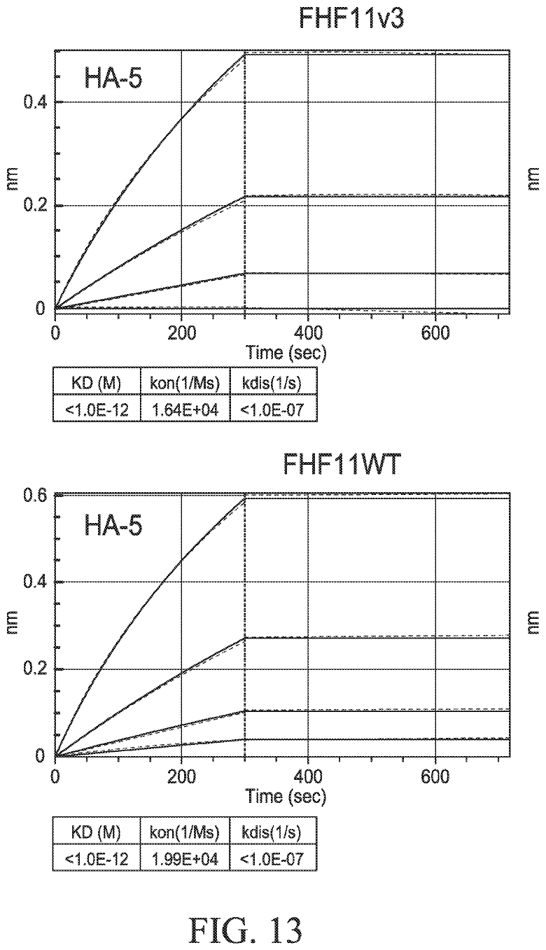

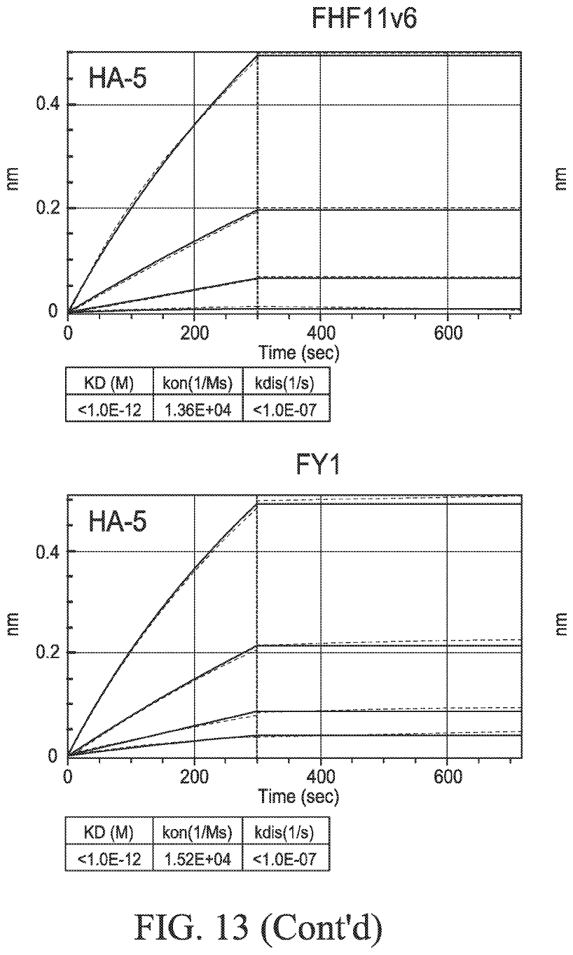

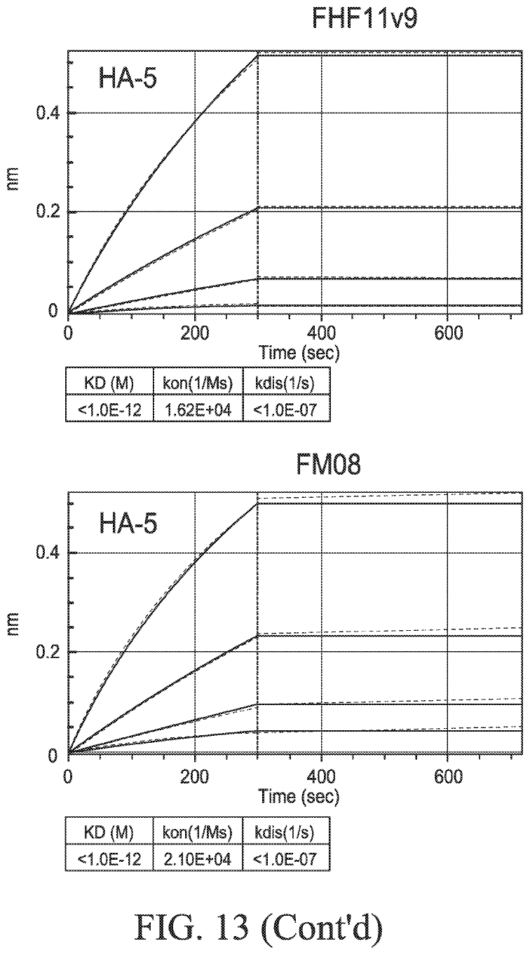

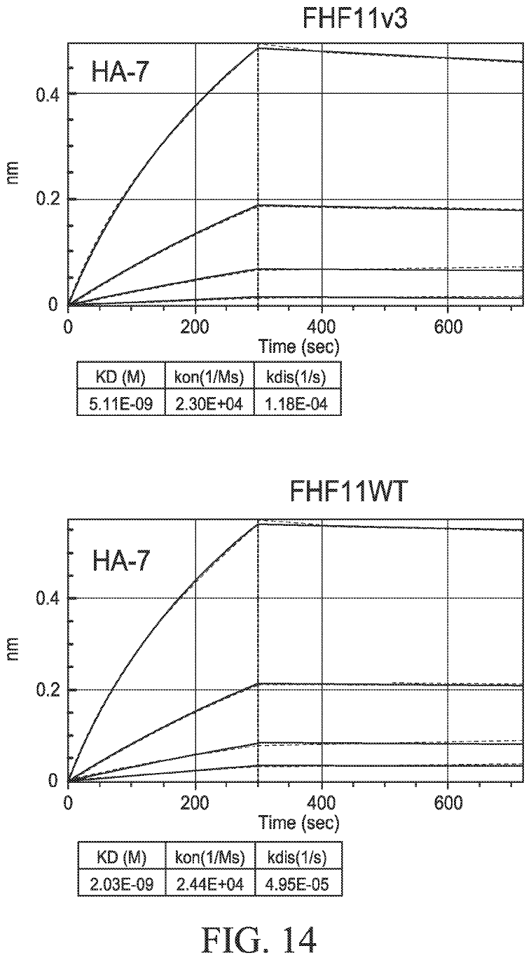

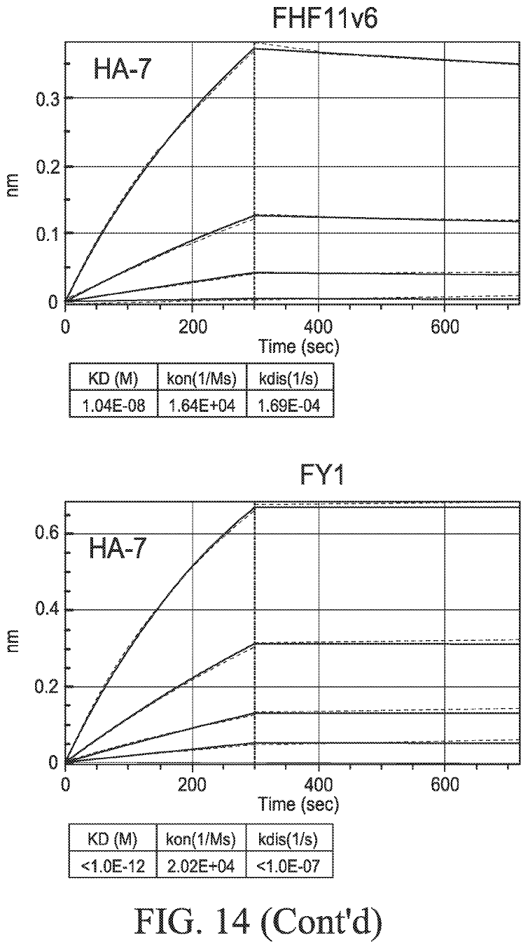

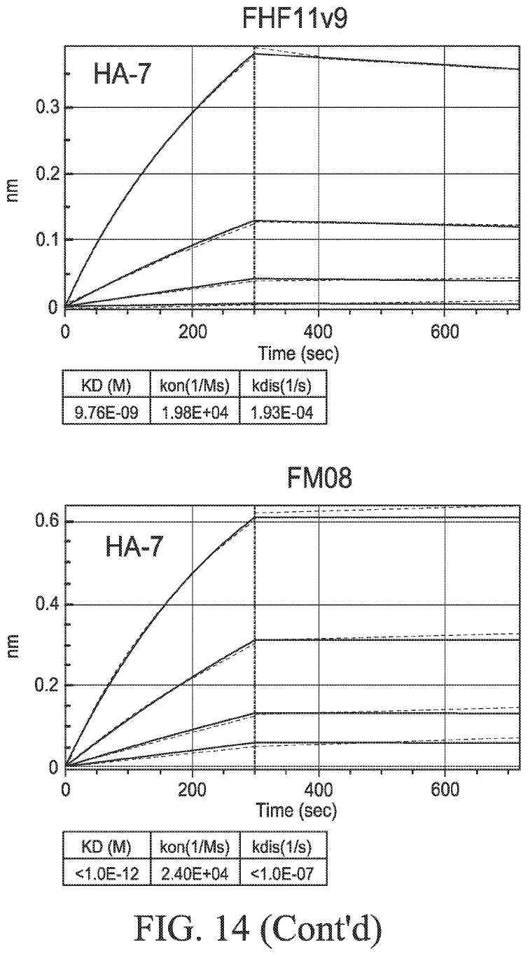

Binding of wild-type FHF11 (“FHF11-WT”) and the fifteen variant antibodies (FHF11v1 to FHF11v15) to cells expressing different HA subtypes derived from viruses circulating in the animal reservoir was measured by FACS (FIG. 10A). FHF11v3 (VH: SEQ ID NO.:31, VL: SEQ ID NO.:8), FHF11v6 (VH: SEQ ID NO.:34, VL: SEQ ID NO.:8), and FHF11v9 (VH: SEQ ID NO.:37, VL: SEQ ID NO.:8) were tested in certain further studies. Binding of these antibodies to multiple HA types was further investigated by ELISA using a panel of H3N2 HAs from human IAV isolates. Results are shown in FIG. 11. Binding to a panel of group I HAs derived from H1N1, H2N2, H5N1, and H9N2 viruses was also tested. Results are shown in FIG. 12. Bio-Layer Interferometry (BLI) was used to determine KD, association (kon), and dissociation (kdis) for FHF11-WT, FHF11v3, FHF11v6, binding to H5 (FIG. 13) and H7 (FIG. 14) antigens.

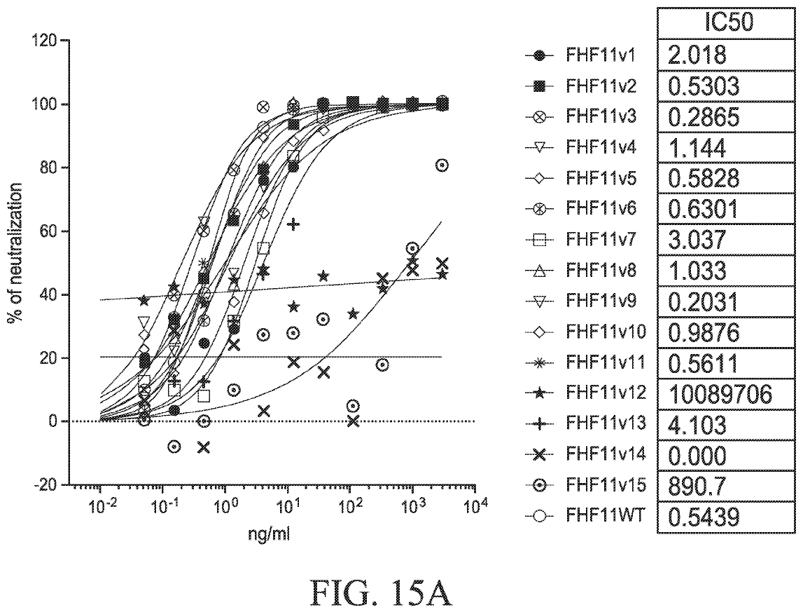

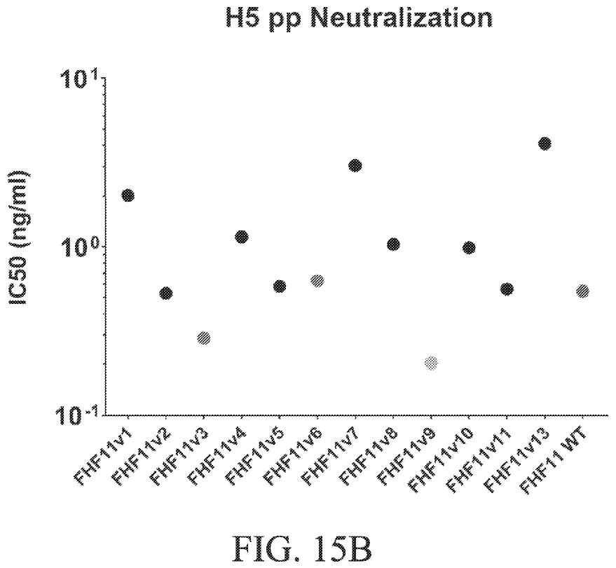

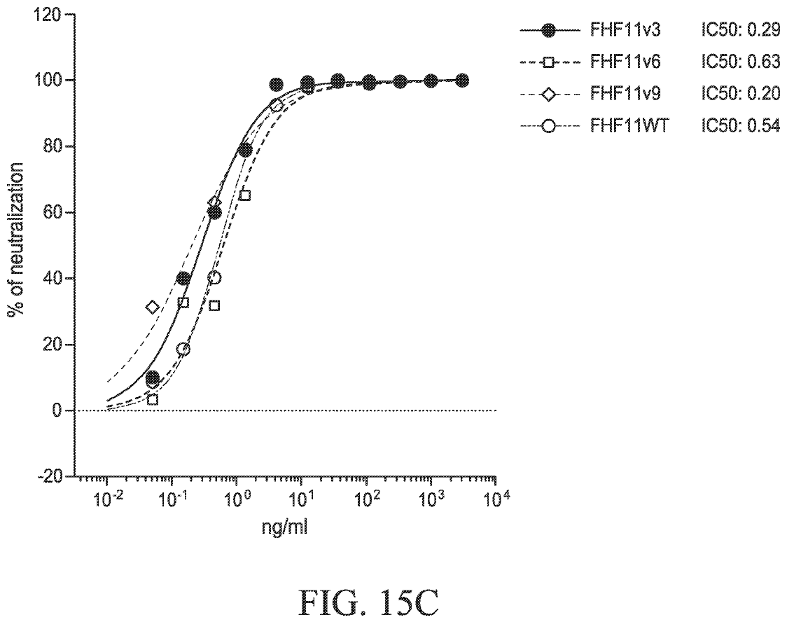

Neutralization of H5 pseudovirus by FHF11-WT and the fifteen variant antibodies generated from FHF11-WT was evaluated. A graph showing percent neutralization at various antibody concentrations (ng/ml) is provided in FIG. 15A, while neutralization, reported as IC50 (ng/ml) values, is shown in FIG. 15B for FHF11-WT and twelve (12) of the variant antibodies. FIG. 15C shows data for FHF11-WT and three variant antibodies, FHF11v3, FHF11v6, and FHF11v9, that were selected for further analysis.

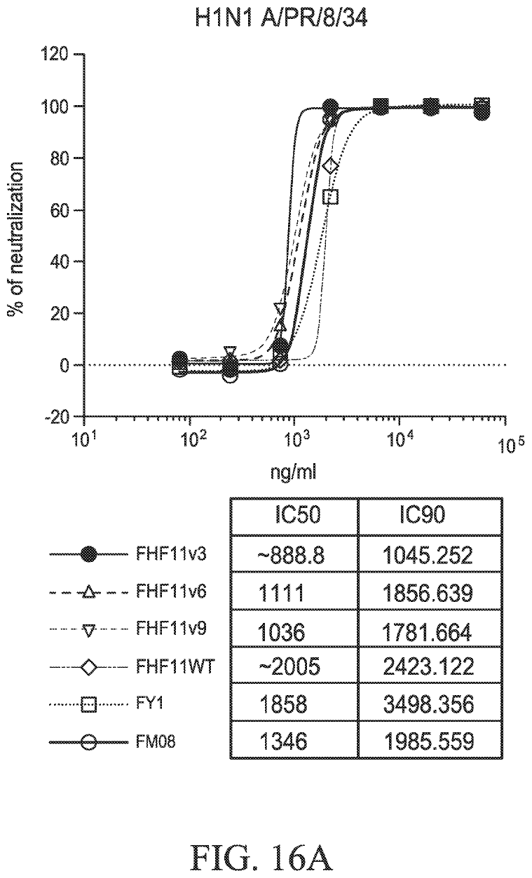

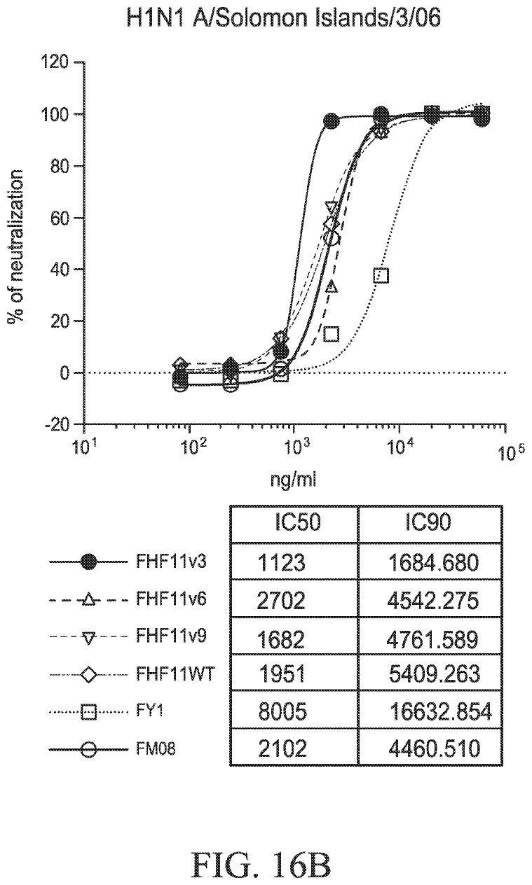

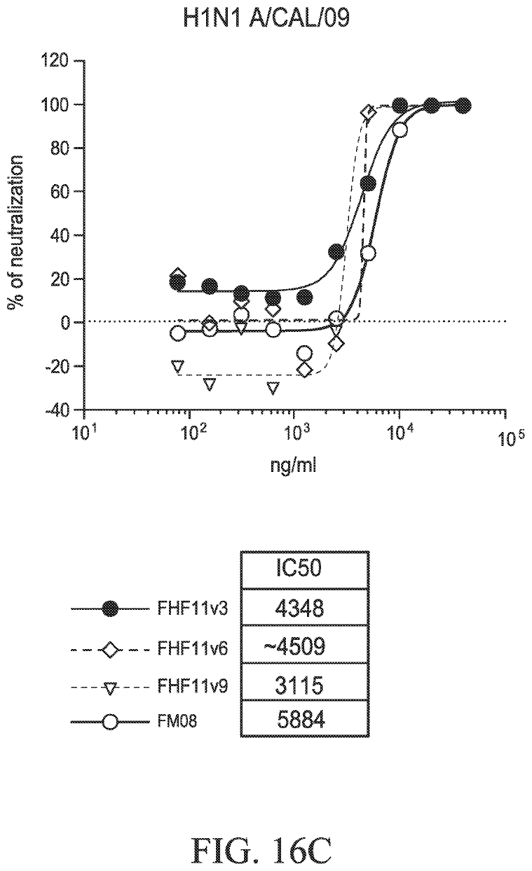

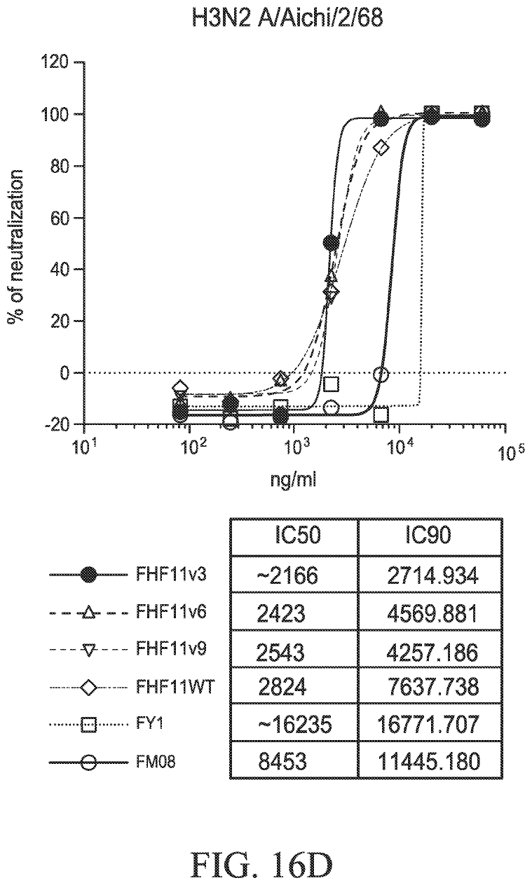

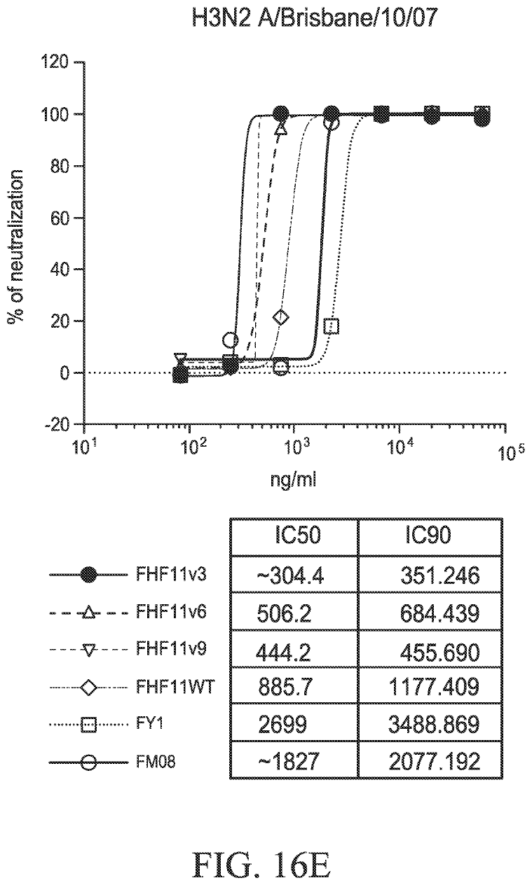

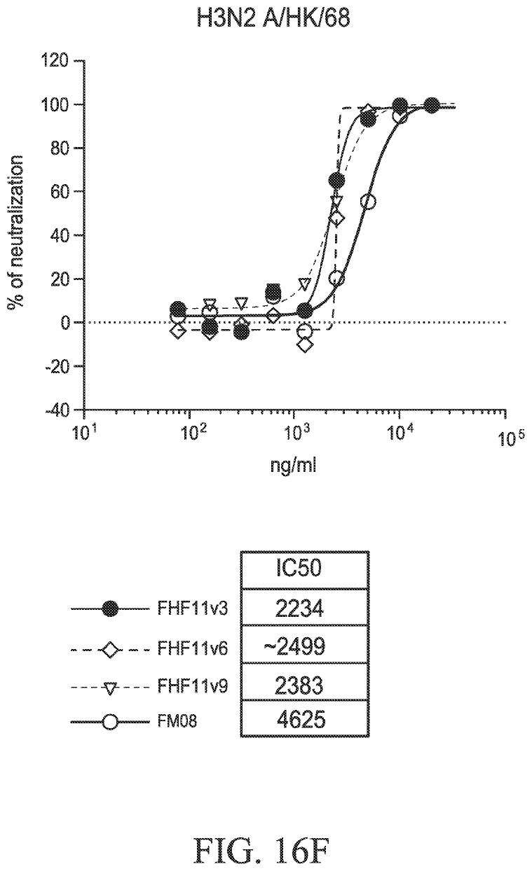

Neutralization of H1N1 and H3N2 subtypes by FHF11-WT, FHF11v3, FHF11v6, and FHF11v9 was evaluated. Influenza subtypes tested were H1N1 A/PR/8/34 (FIG. 16A), H1N1 A/Solomon Islands/3/06 (FIG. 16B), H1N1 A/California/2009 (FIG. 16C), H3N2 A/Aichi/2/68 (FIG. 16D), A/Brisbane/10/07 (FIG. 16E), and H3N2 A/Hong Kong/68 (FIG. 16F).

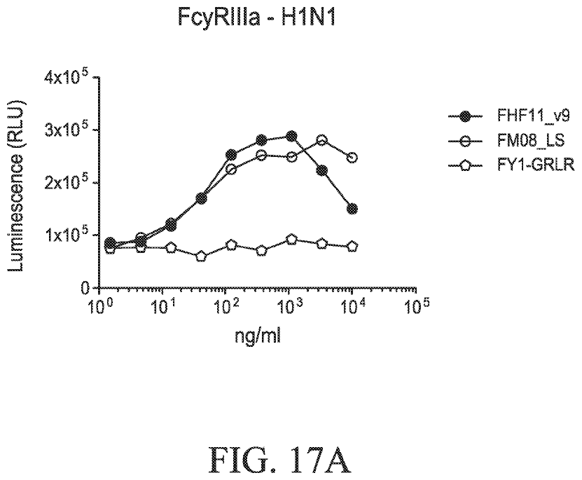

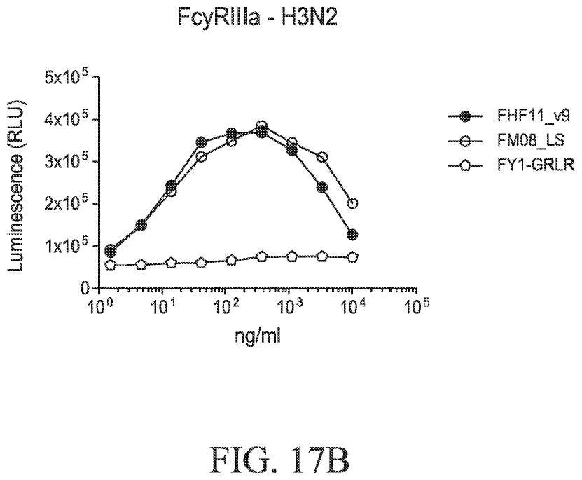

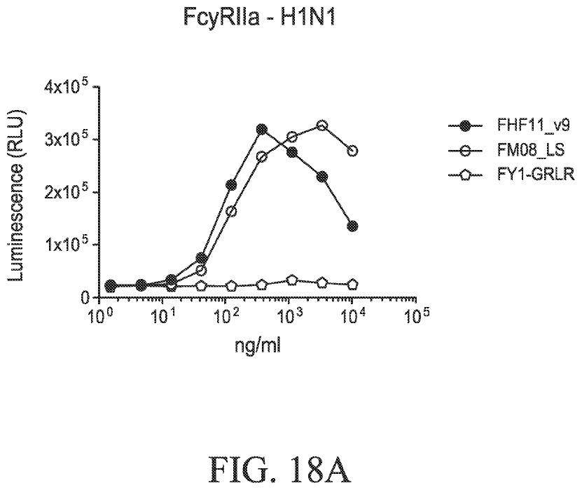

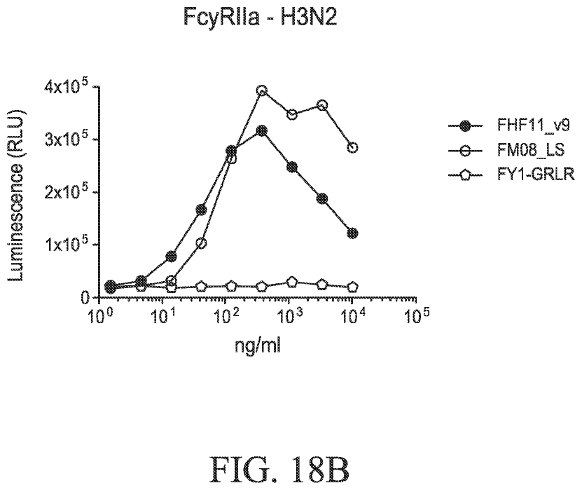

FHF11v9 activation of FcγRIIIa and FcγRIIa was evaluated using a NFAT-mediated luciferase reporter in engineered Jurkat cells. Activation of Jurkat-FcγRIIIa (F158) cells was measured following contact with A549 cells that were pre-infected with H1N1 (FIG. 17A) or H3N2 (FIG. 17B). Activation of Jurkat-FcγRIIa (H131) cells was measured following contact with A549 cells that were pre-infected with H1N1 (FIG. 18A) or H3N2 (FIG. 18B). Activation by comparator antibodies FM08 (comprising M428L/N434S Fc mutations; “FM08_LS” in the figure) and FYl-GRLR (comprising G236R/L328R Fc mutations) were also measured.

### Example 3

### In Vivo Pharmacokinetic and Pharmacodynamic Studies

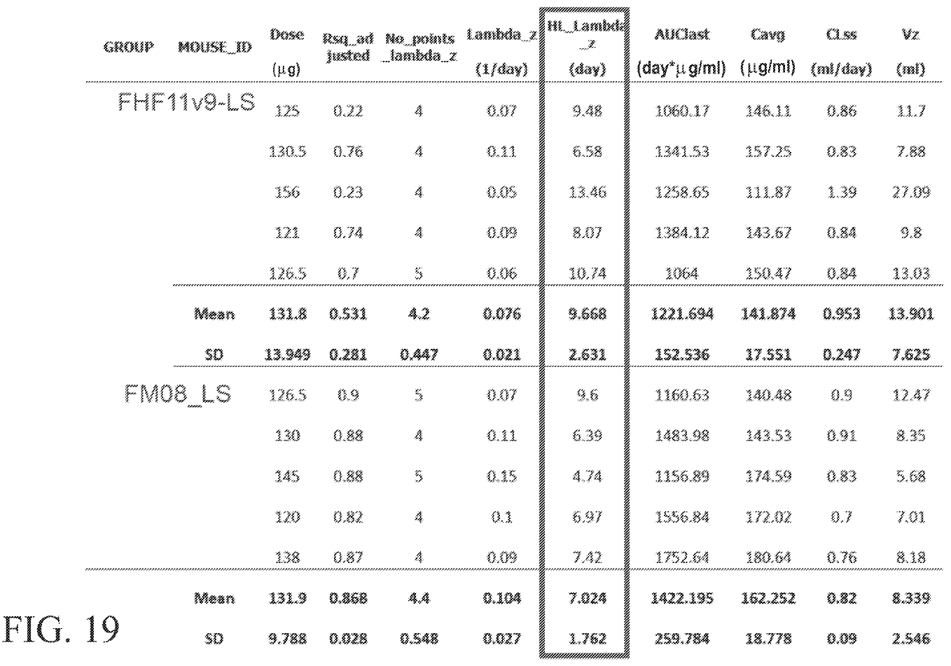

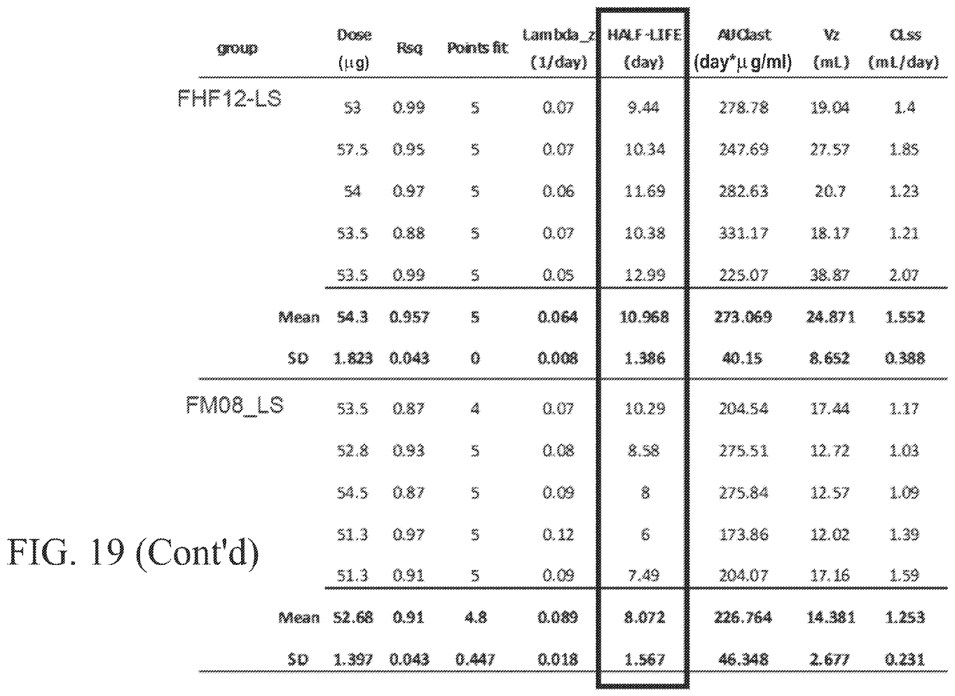

Pharmacokinetic analysis of Fc variants (M428L/N434S mutations) of FHF11v9 (“FHF11v9-LS”), FHF12 (“FHF12-LS”), and a comparator antibody, FM08_LS, was peformed in in tg32 mice, and half-life was determined, as shown in FIG. 19. Plasma concentration of the antibodies was determined in vitro using an ELISA assay. IAV-HA antigen (Influenza A virus H1N1 A/California/07/2009 Hemagglutinin Protein Antigen (with His Tag); Sino Biologicals) was diluted to 2 μg/ml in PBS and 25 μl were added to the wells of a 96-well flat bottom 1%2-area ELISA plate for coating over night at 4° C. After coating, the plates were washed twice with 0.5×PBS supplemented with 0.05% Tween20 (wash solution) using an automated ELISA washer. Then, plates were blocked with 100 μl/well of PBS supplemented with 1% BSA (blocking solution) for 1 h at room temperature (RT) and then washed twice. Plasma samples were centrifuged at 10′000 g for 10 min at 4° C. and then pre-diluted 1:2000 (2 and 6 hrs timepoints), 1:1000 (24 hr timepoint), 1:400 (day 3 and 7 timepoint), and 1:250 (day 10, 14 and 17 timepoints). For anti NA mabs, plasma samples were centrifuged at 10′000 g for 10 min at 4° C. and then pre-diluted 1:150 (2 and 6 hrs timepoints), 1:75 (24 hr timepoint), 1:45 (day 3 timepoint), 1:30 (day 7 timepoint) and 1:15 (day 10, 14 and 17 timepoints) in blocking solution in 96-well cell culture plates. Samples were then diluted 1:2 stepwise in duplicates for a total of 8 dilutions. Standards for each antibody to be tested were prepared similarly via diluting the antibodies to 0.5 μg/ml. Standards were then diluted 1:3 stepwise in blocking solution in duplicates for a total of 8 dilutions. Twenty-five μl of the prepared samples or standards were added to hemagglutinin (HA) or Goat anti human IgG-coated wells and incubated for 1 h at RT. After four washes, 25 μl of goat anti human-IgG HRP conjugate (AffiniPure F(ab′)₂ Fragment, Fcγ Fragment-Specific; Jackson ImmunoResearch) diluted in blocking solution 1:5000 (final concentration 0.16 μg/ml) were added per well for detection and incubated at RT for 1 h. After four washes, plates were developed by adding 25 μl per well of SureBlue TMB Substrate (Bioconcept). After ˜7-20 min incubation at RT, when the color reaction reached a plateau (max OD ˜3.8), 25 μl of 1% HCl were added per well to stop the reaction and absorbance was measured at 450 nm using a spectrophotometer.

To determine the concentration of the antibodies in mouse plasma, OD values from ELISA data were plotted vs. concentration in the Gen5 software (BioTek). A non-linear curve fit was applied using a variable slope model, four parameters and the equation: Y=(A−D)/(1+(X/C)\hat{ }B+D). The OD values of the sample dilutions that fell within the predictable assay range of the standard curve 3/4 as determined in setup experiment by quality control samples in the upper, medium or lower range of the curve 4 were interpolated to quantify the samples. Plasma concentration of the antibodies were then determined considering the final dilution of the sample. If more than one value of the sample dilutions fell within the linear range of the standard curve, an average of these values was used. Pharmacokinetics (PK) data were analyzed by using WINNONLIN NONCOMPARTMENTAL ANALYSIS PROGRAM (8.1.0.3530 Core Version, Phoenix software, Certara) with the following settings: Model: Plasma Data, i.v. Bolus Administration; Number of non-missing observations: 8; Steady state interval Tau: 1.00; Dose time: 0.00; Dose amount: 5.00 mg/kg; Calculation method: Linear Trapezoidal with Linear Interpolation; Weighting for lambda_z calculations: Uniform weighting; Lambda_z method: Find best fit for lambda_z, Log regression. Graphing and statistical analyses (linear regression or outlier analysis) were performed using Prism 7.0 software (GraphPad, La Jolla, CA, USA).

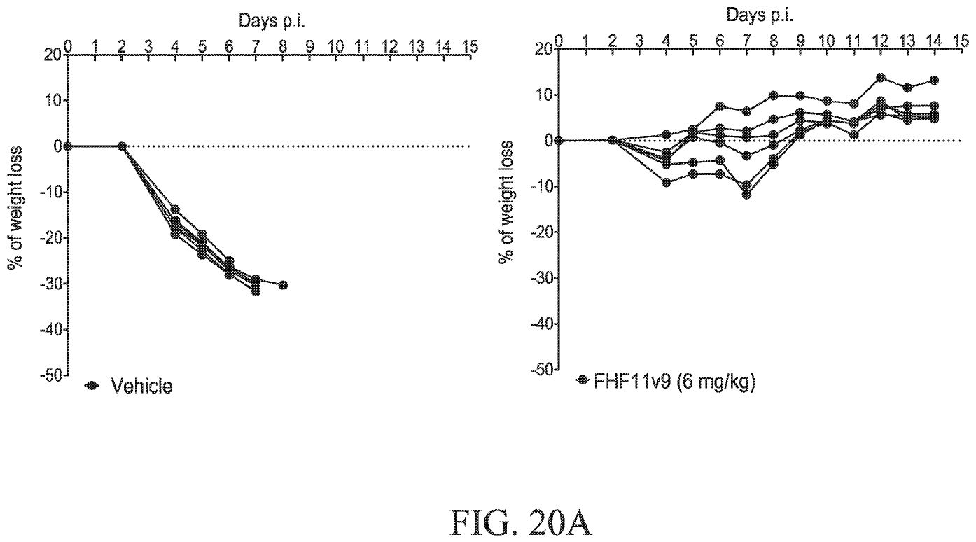

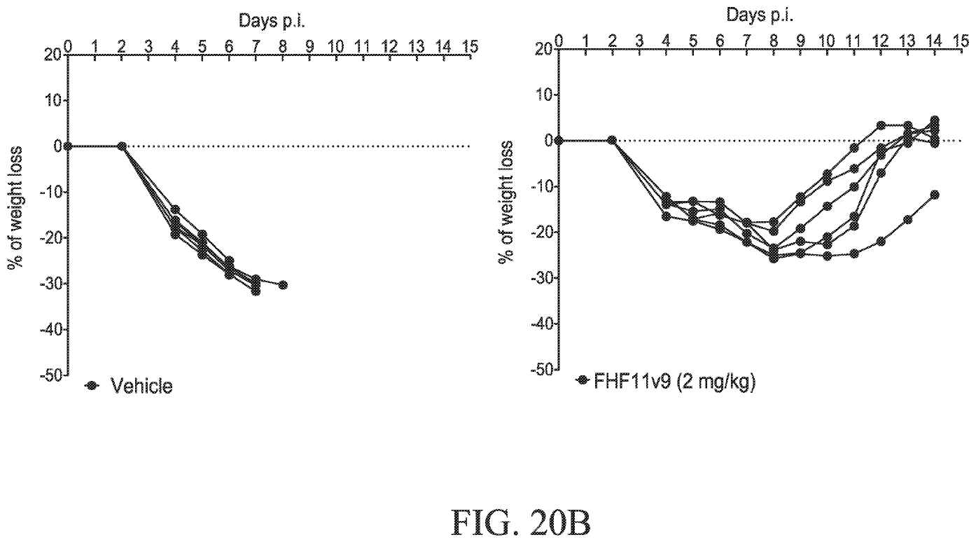

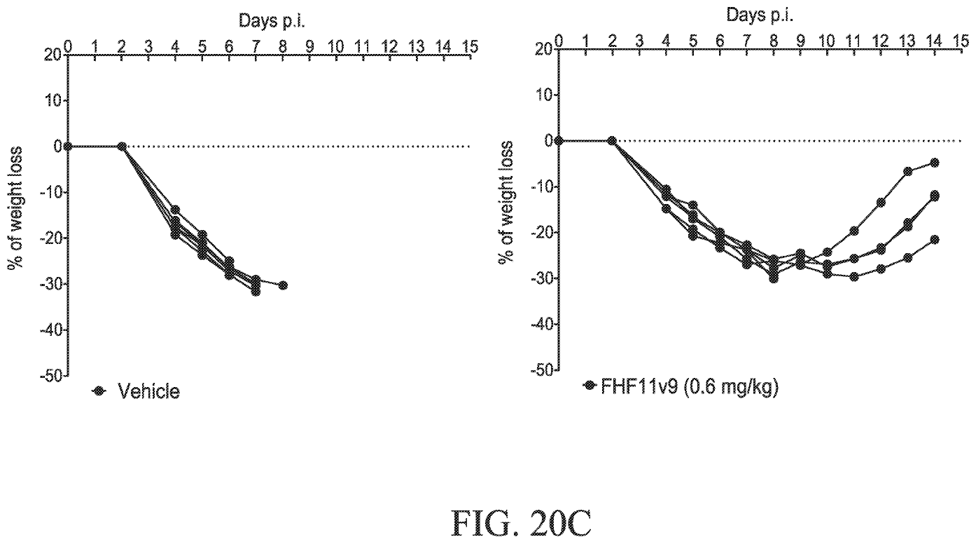

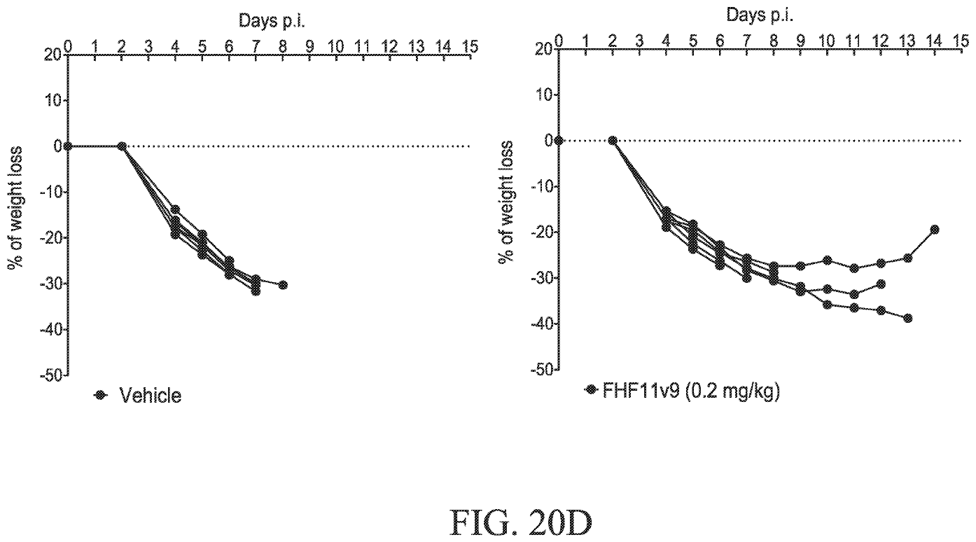

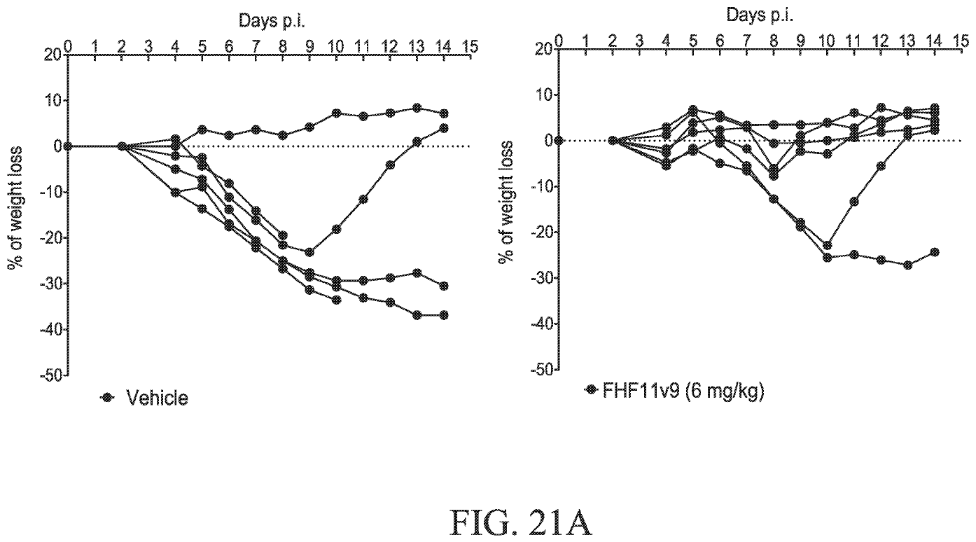

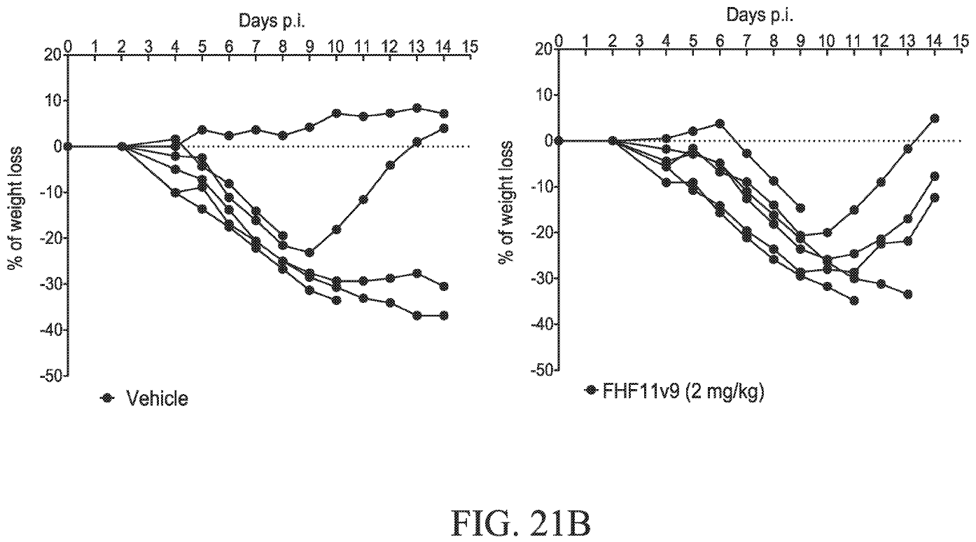

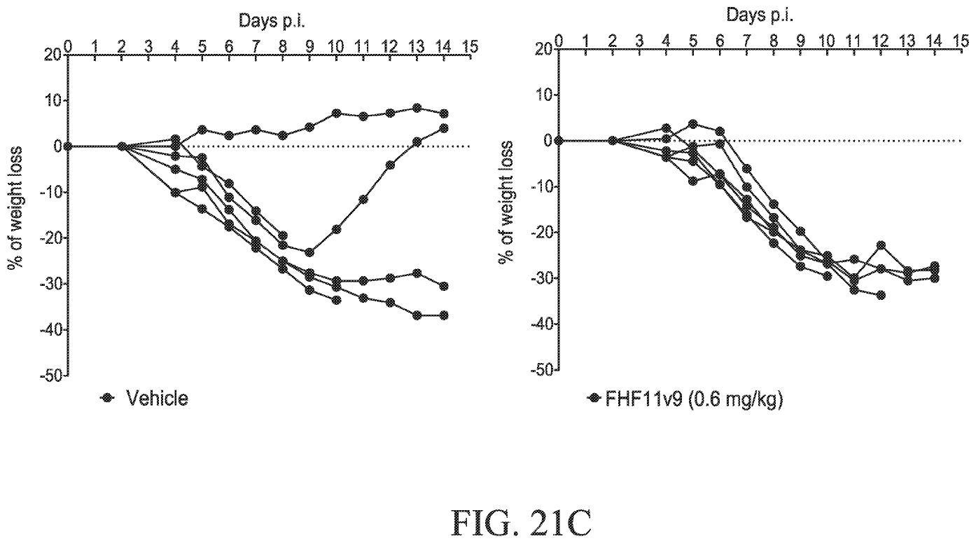

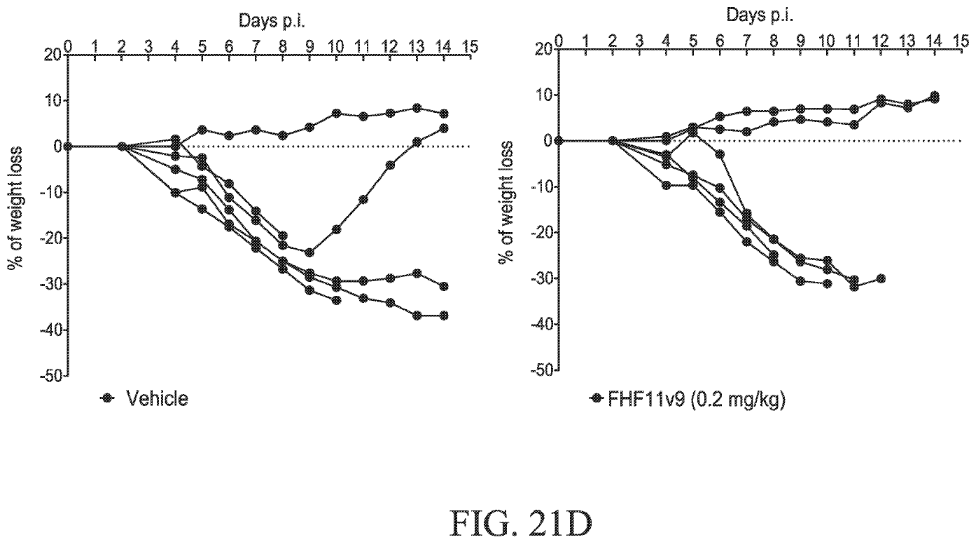

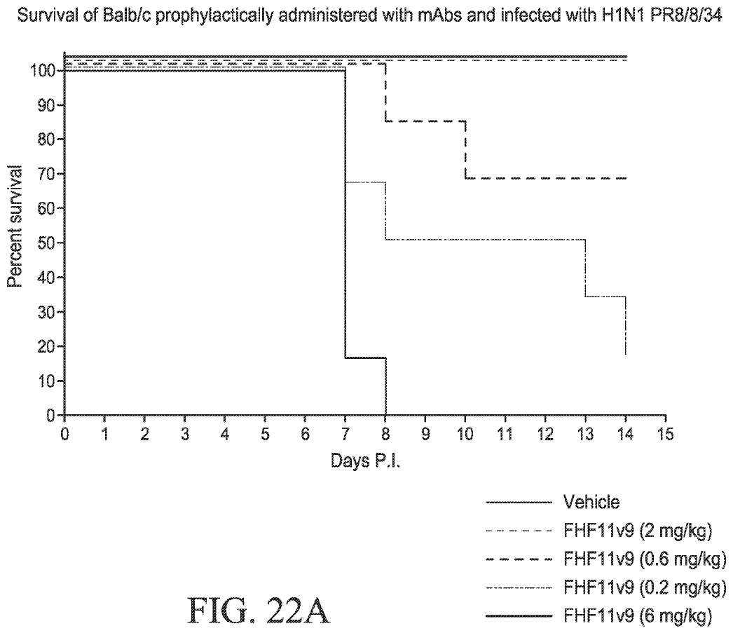

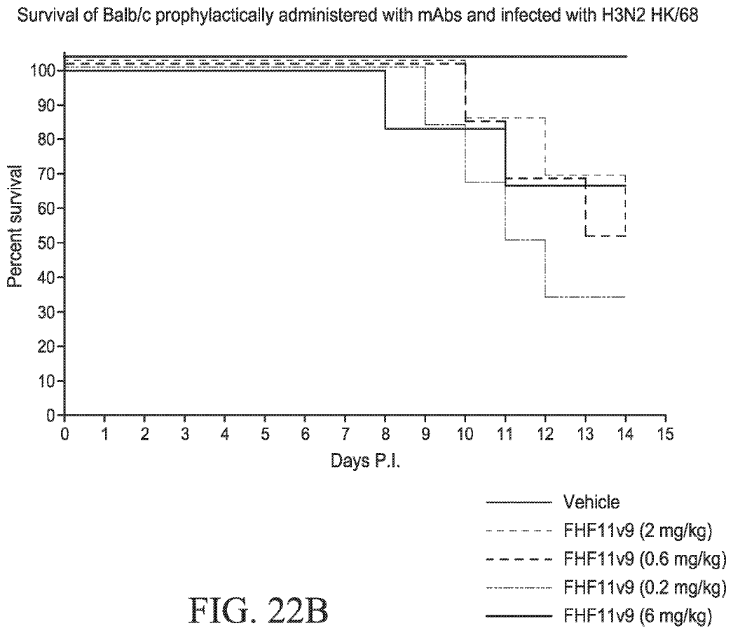

Prophylactic activity of FHF11v9 was evaluated in a murine BALB/c model of IAV infection. Briefly, BALB/c mice, 7-8 weeks of age, were administered (i.v.) FHF11v9 or vehicle control one day prior to intranasal infection at LD90 (90% of a lethal dose) with H1N1 subtype A/Puerto Rico/8/34 or H3N2 subtype A/Hong Kong/1/68. Antibody was administered at 0.2, 0.6, 2, or 6 mg/kg. Baseline serum was collected at the start of infection, and both body weight and mortality were evaluated on each of Days 2-14 post-infection. Body weight measurements over fifteen days are shown in FIGS. 20A-20D (A/Puerto Rico/8/34 administered following FHF11v9) and FIGS. 21A-21D (A/Hong Kong/1/68 administered following FHF11v9). Overall mortality was also measured (FIG. 22A, A/Puerto Rico/8/34-infected mice; FIG. 22B, A/Hong Kong/1/68-infected mice).

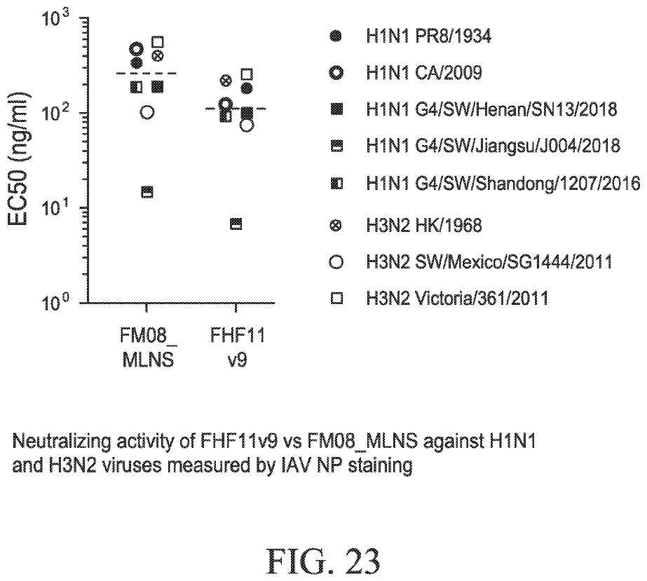

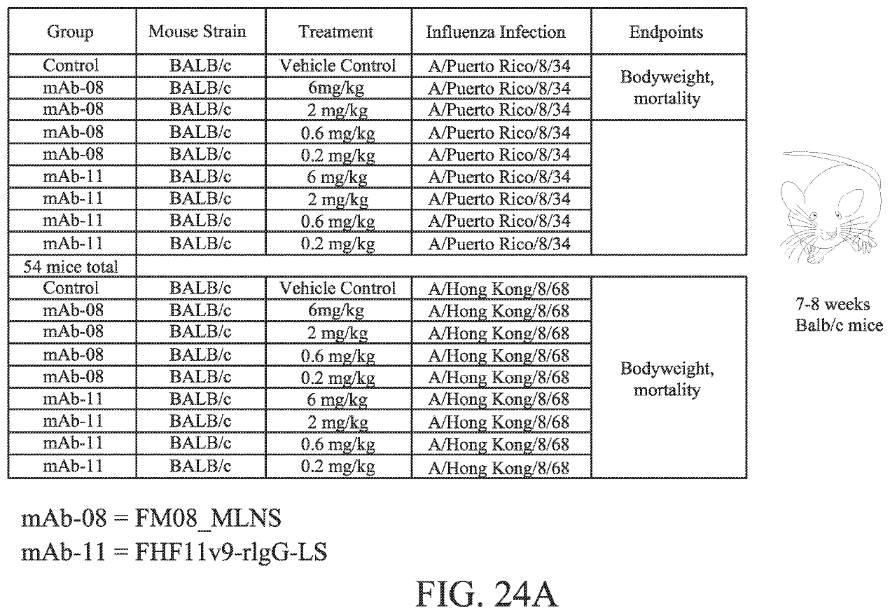

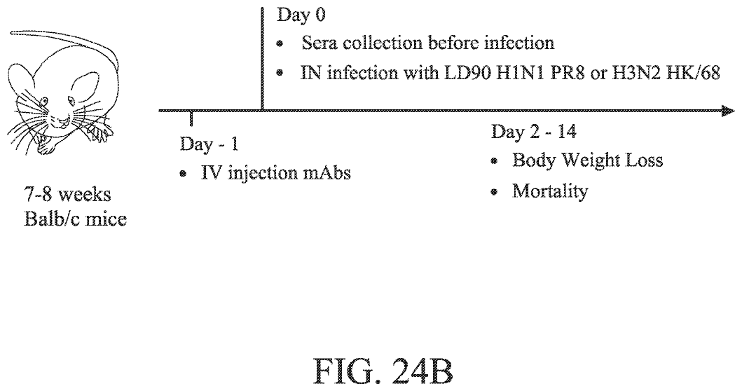

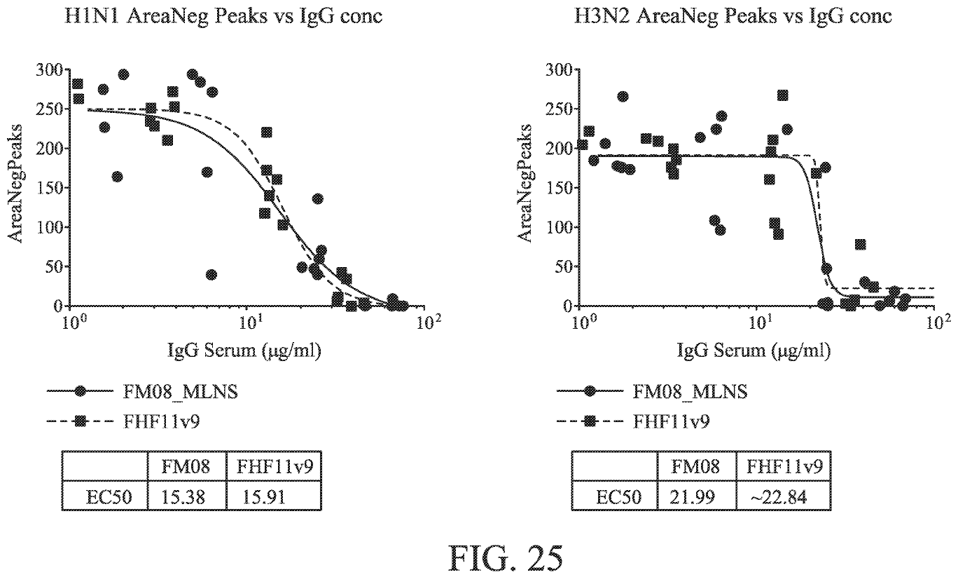

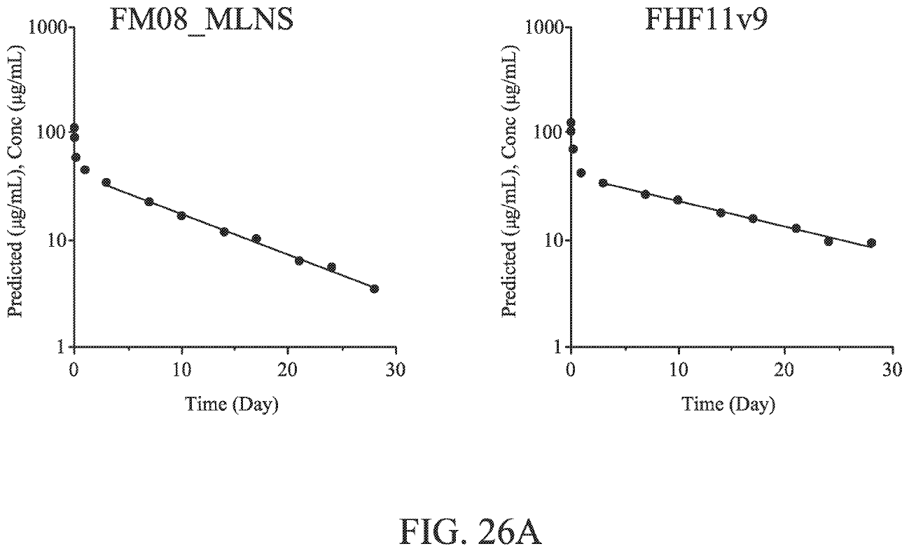

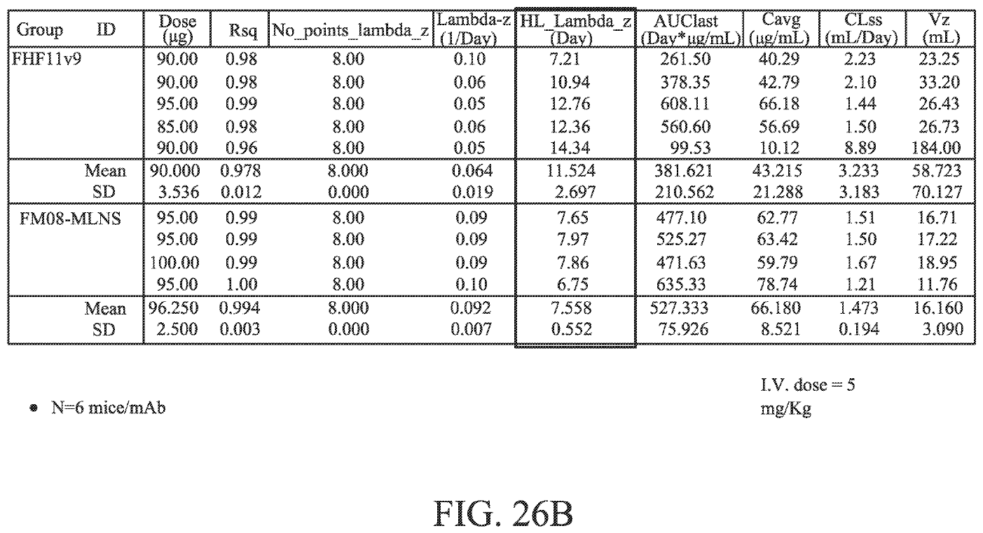

Additional in vitro neutralization and in vivo prophylaxis and pharmacokinetics studies were performed. Data and assay set-ups are shown in FIGS. 23-26B.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/117,437, filed on Nov. 23, 2020 and U.S. Provisional Application No. 63/123,419, filed on Dec. 9, 2020 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody, or antigen-binding fragment thereof, comprising a heavy chain variable domain (VH) comprising a complementarity determining region (CDR)H1, a CDRH2, and a CDRH3, and a light chain variable domain (VL) comprising a CDRL1, a CDRL2, and a CDRL3, wherein:
(i) the CDRH1 comprises or consists of the amino acid sequence of any one of SEQ ID NOs.: 32, 3, or 15, or a functional variant thereof comprising one, two, or three acid substitutions, one or more of which substitutions is optionally a conservative substitution and/or is a substitution to a germline-encoded amino acid; and/or
(ii) the CDRH2 comprises or consists of the amino acid sequence of any one of SEQ ID NOs.: 35, 4, 29, 16, and 42, or a functional variant thereof comprising one, two, or three amino acid substitutions, one or more of which substitutions is optionally a conservative substitution and/or is a substitution to a germline-encoded amino acid; and/or
(iii) the CDRH3 comprises or consists of the amino acid sequence of SEQ ID NO.: 5 or 17, or a functional variant thereof comprising one, two, or three amino acid substitutions, one or more of which substitutions is optionally a conservative substitution and/or is a substitution to a germline-encoded amino acid; and/or
(iv) the CDRL1 comprises or consists of the amino acid sequence of SEQ ID NO.: 9 or 21, or a functional variant thereof comprising one, two, or three amino acid substitutions, one or more of which substitutions is optionally a conservative substitution and/or is a substitution to a germline-encoded amino acid; and/or
(v) the CDRL2 optionally comprises or consists of the amino acid sequence of SEQ ID NO.: 10 or 22, or a functional variant thereof comprising one, two, or three amino acid substitutions, one or more of which substitutions is optionally a conservative substitution and/or is a substitution to a germline-encoded amino acid; and/or
(vi) the CDRL3 comprises or consists of the amino acid sequence of SEQ ID NO.: 11 or 23, or a functional variant thereof comprising having one, two, or three amino acid substitutions, one or more of which substitutions is optionally a conservative substitution and/or is a substitution to a germline-encoded amino acid,
wherein the antibody or antigen-binding fragment is capable of binding to an influenza A virus (IAV) hemagglutinin (HA).

2. The antibody or antigen-binding fragment of claim 1, wherein the antibody or antigen-binding fragment is capable of binding to an influenza A virus (IAV) hemagglutinin (HA on a cell surface of a host cell and/or on a virion.

3. The antibody or antigen-binding fragment of claim 1, which is capable of neutralizing an IAV infection in an in vitro model of infection and/or in an in vivo animal model of infection and/or in a human, wherein, optionally, the in vitro model of infection comprises a target cell and a pseudovirus or a target cell and a live virus.

4. The antibody or antigen-binding fragment of claim 1, comprising CDRH1, CDRH2, CDRH3, CDRL1, CDRL2, and CDRL3 amino acid sequences of SEQ ID NOs.:
(i) 32, 35, 5, and 9-11, respectively;
(ii) 3 (optionally comprised in SEQ ID NO.:53), 29, 5 and 9-11, respectively;
(iii) 32, 4, 5 and 9-11, respectively;
(iv) 3 (optionally comprised in SEQ ID NO.:53), 35, 5 and 9-11, respectively;
(v) 3 (optionally comprised in SEQ ID NO.:53), 4, 5, and 9-11, respectively;
(vi) 15-17 and 21-23, respectively; or
(vii) 15, 42, 17 and 21-23, respectively.

5. (canceled)

6. The antibody or antigen-binding fragment of claim 1, wherein:
(i) the VH comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 37, 2, 26, 28, 31, 34, 14, 39 and 41, wherein sequence variation with reference to SEQ ID NO.: 37, 2, 26, 28, 31, 34, 14, 39 or 41, respectively, is optionally comprised in one or more framework region and/or sequence variation comprises one or more substitution to a germline-encoded amino acid; and/or
(ii) the VL comprises or consists of an amino acid sequence having at least 80% (e.g., 80%, 85%, 90%, 91%, 92%, 93%, 94%, 95%, 96%, 97%, 98%, 99%, or more) identity to the amino acid sequence of any one of SEQ ID NOs.: 8 or 20, wherein sequence variation with respect to SEQ ID NO.:8 or 20, respectively, is optionally comprised in one or more framework regions and/or sequence variation comprises one or more substitution to a germline-encoded amino acid.

7. (canceled)

8. The antibody or antigen-binding fragment of claim 1, wherein the VH and the VL comprise or consist of the amino acid sequences according to SEQ ID NOs.:
(i) 37 and 8, respectively;
(ii) 26 and 8, respectively;
(iii) 28 and 8, respectively;
(iv) 31 and 8, respectively;
(v) 34 and 8, respectively;
(vi) 2 and 8, respectively;
(vii) 14 and 20, respectively;
(viii) 39 and 20, respectively; or
(ix) 41 and 20, respectively.

9.-17. (canceled)

18. An antibody, or antigen-binding fragment thereof,
comprising a heavy chain variable domain (VH) and a light chain variable domain (VL), wherein:
(i) the VH comprises a CDRH1, a CDRH2, and a CDRH3 according to the VH amino acid sequence set forth in any one of SEQ ID NOs.: 37, 2, 26, 28, 31, 34, 14, 39 and 41; and
(ii) the VL comprises a CDRL1, a CDRL2, and a CDRL3 according to the VL amino acid sequence set forth in SEQ ID NO.:20
wherein the antibody or antigen-binding fragment is capable of binding to an influenza A virus (IAV) hemagglutinin (HA).

19. An antibody, or antigen-binding fragment thereof, comprising a heavy chain variable domain (VH) and a light chain variable domain (VL), wherein:
(i) the VH comprises a CDRH1, a CDRH2, and a CDRH3 according to the VH amino acid sequence set forth in any one of SEQ ID NOs.: 37, 2, 26, 28, 31, 34, 14, 39 and 41; and
(ii) the VL comprises a CDRL1, a CDRL2, and a CDRL3 according to the VL amino acid sequence set forth in SEQ ID NO.:8,
wherein the antibody or antigen-binding fragment is capable of binding to an influenza A virus (IAV) hemagglutinin (HA).

20. The antibody or antigen-binding fragment of claim 19, wherein the CDRs are according to the IMGT numbering system, the Kabat numbering system, the Chothia numbering system, the AHo numbering system, the North numbering system, or the Martin numbering system.

21.-35. (canceled)

36. The antibody or antigen-binding fragment of claim 1, wherein:
(1) the antibody or antigen-binding fragment is capable of preventing and/or attenuating an infection by:
(i) a H1N1 IAV, wherein, optionally, the H1N1 IAV comprises A/PR8/34; and/or
(ii) a H3N2 IAV, wherein, optionally, the H3N2 IAV comprises A/Hong Kong/68;
(2) the antibody or antigen-binding fragment is capable of preventing or reducing weight loss in a subject having an IAV infection, optionally for (i) up to 15 days, or (ii) for 15 or more days, following administration of an effective amount of the antibody or antigen-binding fragment, wherein preventing or reducing weight loss is with reference to an untreated reference subject having the IAV infection;
(3) the antibody or antigen-binding fragment is capable of preventing a loss in body weight of greater than 10% in a subject having an IAV infection, wherein a loss in body weight is determined by reference to the subject's body weight just prior to or in an early stage of the IAV infection;
(4) the antibody or antigen-binding fragment is capable of extending survival of a subject having an IAV infection, as compared to survival of an untreated reference subject having the IAV infection;
(5) the antibody or antigen-binding fragment has an in vivo half-life in a mouse (e.g., a tg32 mouse);
(i) in a range of: from about 7 days to about 12.2 days, from about 8 days to about 11 days, from about 8.5 days to about 10.5 days, or from about 9 days to about 10.5 days;
(ii) of between 8 days and 11 days, or between 8.5 days and 10.5 days, or between 9 days and 10 days;
(iii) of 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, or 12.2 days;
(iv) in a range of from about 9.5 days to about 12.5 days, from about 10 days to 11.5 days;
(v) of from 10 days to 11 days, or from 10.5 days to 11 days;
(vi) between 10 days and 11.5 days, or between 10.5 days and 11 days, or between 10 days and 11 days; and/or
(vii) of 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, or 12.5 days;
(6) the antibody or antigen-binding fragment is capable of binding to any one or more of the following IAV subtypes: H1, H2, H3, H4, H5, H8, H9, H10, H11, H12, H13, H14, H15, H17, and H18;
(7) the antibody or antigen-binding fragment is capable of preventing or attenuating an IAV infection a subject;
(8) the antibody or antigen-binding fragment is capable of neutralizing infection by:
(i) a H1N1 IAV, wherein, optionally, the H1N1 IAV comprises any one or more of: A/California/07/2009, A/PR/8/34, and A/Solomon Islands/3/06; and
(ii) a H3N2 IAV, wherein, optionally, the H3N2 IAV comprises any one or more of: A/Aichi/2/68, A/Brisbane/10/07, and A/Hong Kong/68:
(9) the antibody or antigen-binding fragment is capable of neutralizing infection by:
(i) a Group 1 IAV, wherein, optionally, the Group 1 IAV comprises or is a H5 IAV, wherein, further optionally, the H5 IAV comprises or is H5/VN/11/94 pp; and
(ii) a Group 2 IAV, wherein, optionally, the Group 2 IAV comprises or is a H7 IAV, wherein, further optionally, the H7 IAV comprises or is H7/IT/99 pp,
wherein, optionally, neutralization of infection is as determined using a virus pseudotyped with the IAV;
(10) the antibody or antigen-binding fragment is capable of activating a human FcγRIIIa, which is optionally a F158 allele; and/or
(11) the antibody or antigen-binding fragment is capable of activating a human a human FcγRIIa, which is optionally a H131 allele;
(12) the antibody or antigen-binding fragment is capable of binding to any one or more of the following H3N2 IAV subtypes: A/Babol/36/2005: A/Hong Kong/CUHK31987/2011: A/Texas/50/2012; A/Wisconsin/67/2005; A/Netherlands/178/1995; A/Johannesburg/33/1994: A/Guangdong-Luohu/1256/2009; A/California/7/2004: A/Hanoi/EL134/2008; A/Wuhan/359/1995: A/Victoria/210/2009; A/Philippines/472/2002: A/Hanoi/EL201/2009: AVictoria/210/2009: A/Missouri/09/2014: A/Perth/16/2009; A/Wyoming/03/2003; A/Moscow/10/1999; A/Sydney/5/1997: A/Nanchang/933/1995; A/Beijing/32/92; A/Aichi/2/1968; A/Brisbane/10/2007; and A/Switzerland/9715293/2013;
(13) the antibody or antigen-binding fragment is capable of binding to one or more of (i)-(iv):
(i) a H1 HA, which optionally comprises any one or more of: A/England/195/2009: A/Brisbane/59/2007: A/Solomon Islands/3/2006: A/New Caledonia/20/99; A/Texas/36/1991; A/Taiwan/01/1986; A/New Jersey/8/1976; A/Albany/12/1951; A/Fort Monmouth/1/1947; A/New York/1/1918: A/Puerto Rico/8/34; and A/California/07/2009;
(ii) a H2 HA, optionally comprising A/Japan/305/1957;
(iii) a H5 HA, optionally comprising A/Vietnam/1194/2004; and
(iv) a H9 HA, optionally comprising A/Hong Kong/1073/99; and/or
(14) the antibody or antigen-binding fragment binds to H5 HA and/or to H7 HA with a KD of less than 1.0E-12 M, less than 1.0E-11 M, less than 1.0E-10 M, less than 1.0E-9 M, less than 1.0E-8 M, or less than 1.0E-7 M, or of 1.0E-8M or less, of 1.0E-9M or less, of 1.0E-10 or less, of 1.0E-11 or less, or 1.0E-12 or less (e.g., as determined by Bio-Layer Interferometry (BLI)).

37.-64. (canceled)

65. The antibody or antigen-binding fragment of claim 1, which is a IgG, IgA, IgM, IgE, or IgD isotype.

66. The antibody or antigen-binding fragment of claim 1, which is an IgG isotype selected from IgG1, IgG2, IgG3, and IgG4.

67. The antibody or antigen-binding fragment of claim 1, which is human, humanized, or chimeric, and/or
comprises a human antibody, a monoclonal antibody, a purified antibody, a single chain antibody, a Fab, a Fab′, a F(ab′)2, or a Fv, such as a scFv.

68. (canceled)

69. The antibody or antigen-binding fragment of claim 1, wherein the antibody or antigen-binding fragment is a multi-specific antibody or antigen binding fragment.

70. (canceled)

71. The antibody or antigen-binding fragment of claim 1, further comprising a Fc polypeptide or a fragment thereof, wherein, optionally, the Fc polypeptide or fragment thereof is an IgG1 isotype.

72. The antibody or antigen-binding fragment of claim 71, wherein the Fc polypeptide or fragment thereof comprises:
(i) a mutation that extends in vivo half-life of the antibody or antigen-binding fragment, as compared to the antibody or antigen-binding fragment comprising a reference (e.g., native of a same isotype) Fc polypeptide or fragment thereof that does not comprise the mutation; and/or
(ii) a mutation that increases binding affinity to a human FcγR (e.g., a FcγRIIa and/or a FcγRIIIa), as compared to a reference Fc polypeptide that does not comprise the mutation.

73. The antibody or antigen-binding fragment of claim 72, wherein the mutation that extends in vivo half-life of the antibody or antigen-binding fragment comprises: M428L; N434S; N434H; N434A; N434S; M252Y; S254T; T256E; T250Q; P2571; Q311I; D376V; T307A; E380A; or any combination thereof,
wherein Fc amino acid numbering is according to the EU numbering system.

74. The antibody or antigen-binding fragment of claim 72, wherein the mutation that extends in vivo half-life of the antibody or antigen-binding fragment comprises:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A; or
(viii) any combination of (i)-(vii).

75. (canceled)

76. The antibody or antigen-binding fragment of claim 72, wherein the mutation that enhances binding to a FcγR comprises S239D; 1332E; A330L; G236A; or any combination thereof,
wherein Fc amino acid numbering is according to the EU numbering system.

77. The antibody or antigen-binding fragment of claim 72, wherein the mutation that enhances binding to a FcγR comprises:
(i) S239D/I332E;
(ii) S239D/A330L/I332E;
(iii) G236A/S239D/I332E; or
(iv) G236A/A330L/I332E, optionally not comprising S239D, further optionally comprising a S at position 239.

78.-81. (canceled)

82. An isolated polynucleotide encoding the antibody or antigen-binding fragment of claim 1, or encoding a VH, a heavy chain, a VL, and/or a light chain of the antibody or the antigen-binding fragment.

83. The polynucleotide of claim 82, wherein the polynucleotide comprises deoxyribonucleic acid (DNA) or ribonucleic acid (RNA), wherein the RNA optionally comprises messenger RNA (mRNA).

84.-90. (canceled)

91. A recombinant vector comprising the polynucleotide of claim 82.

92. A host cell comprising the polynucleotide of claim 82, wherein the polynucleotide is heterologous to the host cell and wherein the host cell is capable of expressing the encoded antibody or antigen-binding fragment.

93. (canceled)

94. A composition comprising:
the antibody or antigen-binding fragment of claim 1;
and a pharmaceutically acceptable excipient, carrier, or diluent.

95.-96. (canceled)

97. A method of making an antibody or antigen-binding fragment of any one of claim 1, comprising culturing a host cell comprising a polynucleotide that encodes the antibody or antigen-binding fragment for a time and under conditions sufficient for the host cell to express the antibody or antigen-binding fragment.

98. (canceled)

99. A method of treating or preventing an influenza A virus infection in a subject, the method comprising administering to the subject an effective amount of:
the antibody or antigen-binding fragment of claim 1.

100.-113. (canceled)
